(19) United States
(12) Patent Application Publication
(10) Pub. No.: US 2026/0062668 A1
(43) Pub. Date: Mar. 5, 2026

(54) CLICK CHEMISTRY LIGAND

(71) Applicant: Northeastern University, Boston, MA (US)

(72) Inventors: Sara H. Rouhanifard, Chestnut Hill, MA (US); Keqing Nian, Boston, MA (US)

(21) Appl. No.: 19/251,677

(22) Filed: Jun. 26, 2025

Related U.S. Application Data
(63) Continuation-in-part of application No. 19/131,506.
(60) Provisional application No. 63/426,947, filed on Nov. 21, 2022.
(60) Provisional application No. 63/518,248, filed on Aug. 8, 2023.
(60) Provisional application No. 63/664,711, filed on Jun. 26, 2024.
(60) Provisional application No. 63/768,874, filed on Mar. 7, 2025.

(51) Int. Cl. C12N 5/00; B01J 23/72; C09B 41/00; C12N 15/88

(52) U.S. Cl. CPC C12N 5/0006; B01J 23/72; C09B 41/006; C12N 15/88

(57) ABSTRACT

The disclosure herein provides, in example embodiments, methods and systems for labeling biomolecules with CuAAC using a copper-catalyzed azide-alkyne cycloaddition (CuAAC)-accelerating ligand.

### RELATED APPLICATIONS

This application is a Continuation-in-Part of U.S. application Ser. No. 19/131,506, filed on May 20, 2025, which is the U.S. National Stage of International Application No. PCT Application No. PCT/US2023/079299, filed Nov. 9, 2023, which claims the benefit of U.S. Provisional Application No. 63/426,947, filed on Nov. 21, 2022 and U.S. Provisional Application 63/518,248, filed on Aug. 8, 2023. This application also claims the benefit of U.S. Provisional Application No. 63/664,711, filed Jun. 26, 2024, and U.S. Provisional Application No. 63/768,874, filed Mar. 7, 2025. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 52002353-006 SL.xml; created Jun. 26, 2025, 19,653 Bytes in size.

### BACKGROUND

Of the various conjugation strategies for cellular biomolecules, copper (Cu(I))-catalyzed azide-alkyne cycloaddition (CuAAC) is a preferred click chemistry approach due to its fast reaction rate and the commercial availability of a wide range of conjugates. While extracellular labeling of biomolecules using CuAAC has been widely adopted, intracellular labeling in live cells has been challenging as the high copper concentration required for CuAAC reaction can be toxic to biological systems.

There is a need to develop compositions and methods for copper (Cu(I))-catalyzed azide-alkyne cycloaddition (CuAAC), wherein the CuAAC may be performed in live cells (i.e., intracellularly) or on the surface of live cells.

### SUMMARY

In one aspect, the disclosure provides a system for performing Cu(I)-catalyzed azide-alkyne cycloaddition (CuAAC) inside or on a surface of a cell, comprising: a CuAAC-accelerating ligand comprising a polynucleotide and one or more copper chelators; and a delivery reagent.

In some embodiments, the cell is a live cell. In some embodiments, the delivery reagent comprises a transfection reagent, a reversible pore-forming toxin, or both. In some embodiments, the transfection reagent comprises a lipofection reagent. In some embodiments, the reversible pore-forming toxin comprises Streptolysin-O (SLO). In some embodiments, the polynucleotide of the ligand is hybridized to an oligonucleotide. In some embodiments, the oligonucleotide is complementary to at least a portion of the polynucleotide of the ligand.

In some embodiments, the system further comprises a target comprising an alkyne-containing compound. In some embodiments, the alkyne-containing compound is selected from: an alkyne-derivatized thymidine analog; an alkyne-derivatized uridine analog; an alkyne-derivatized methionine analog; an alkyne-derivatized puromycin analog; an alkyne-derivatized monosaccharide; an alkyne-derivatized choline; a 5′ alkyne DNA probe; a library of alkyne-containing compounds; or any combination of the foregoing.

In some embodiments, the alkyne-derivatized thymidine analog comprises 5-ethynyl-2′-deoxyuridine (EdU); the alkyne-derivatized uridine analog comprises 5-ethynyl uridine (EU); the alkyne-derivatized methionine analog comprises L-homopropargyl (L-HPG); the alkyne-derivatized puromycin analog comprises O-propargylpuromycin (OPP); the alkyne-derivatized monosaccharide comprises N-(4-pentynoyl) mannosamine (Ac₄MaNAl); the alkyne-derivatized choline comprises propargyl choline; or any combination of the foregoing.

In some embodiments, the system further comprises a payload comprising an azide. In some embodiments, the payload comprises a detectable molecule. In some embodiments, the payload comprises a fluorogenic azide.

In some embodiments, the system further comprises sodium ascorbate.

In some embodiments, the system further comprises a nucleic acid splint.

In some embodiments, the polynucleotide of the ligand: comprises a single-stranded DNA, a single-stranded RNA, a single-stranded XNA, a single-stranded modified polynucleotide, an aptamer, or a combination thereof; is complementary to one or more other polynucleotides selected from: a template, a target, a payload, and a combination thereof; binds a polypeptide; is conjugated to at least one of: a nanoparticle, a small molecule, a liposome, an antibody or antigen-binding fragment thereof, a detectable molecule, and a combination thereof; or any combination of the foregoing.

In some embodiments, the one or more copper chelators of the ligand comprise 2-(4-((Bis((1-(tert-butyl)-1H-1,2,3-triazol-4-yl)methyl)amino)methyl)-1H-1,2,3-triazol-1-yl) (BTT).

In some embodiments, the ligand comprises BTT-DNA or BTT_{(1,2)}-DNA.

In another aspect, the disclosure provides a method of performing a Cu(I)-catalyzed azide-alkyne cycloaddition (CuAAC) inside or on a surface of a cell, comprising contacting the cell with the system of claim 1. In some embodiments, the cell is contacted with, in order:
the delivery reagent, wherein the delivery reagent comprises a reversible pore-forming toxin; a payload comprising an azide and a target comprising an alkyne-containing compound; the ligand; and sodium ascorbate;
the target comprising the alkyne-containing compound; the delivery agent, wherein the delivery agent comprises the reversible pore-forming toxin; the payload comprising the azide; the ligand; and sodium ascorbate; and
the ligand, wherein the polynucleotide of the ligand is hybridized to an oligonucleotide; the delivery reagent, wherein the delivery reagent comprises a transfection reagent, and wherein the transfection agent is complexed with the ligand hybridized to the oligonucleotide; the delivery agent, wherein the delivery agent comprises the reversible pore-forming toxin; the payload comprising the azide; the target comprising the alkyne-containing compound; and sodium ascorbate.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1E. Synthesis and characterization of BTT_{(1,2)}-DNA ligand. FIG. 1A depicts a strategy to synthesize BTT_{(1,2)}-DNA ligand (BTT₍₁₎-DNA ligand and BTT₍₂₎-DNA ligand) via copper (Cu(I))-catalyzed azide-alkyne cycloaddition (CuAAC) using S1 (N,N-bis((1-tert-butyl-1H-1,2,3-triazol-4-yl)methyl) prop-2-yn-1-amine). FIG. 1B shows 15% tris/boric acid/EDTA (TBE)-Urea gel electrophoresis for BTT_{(1,2)}-DNA ligand. Ladder (leftmost lane) bands are labeled in base pairs (bp). FIG. 1C shows the relative efficacies of BTT₍₁₎-DNA and BTT₍₂₎-DNA ligands in accelerating CuAAC. An alkyne-labeled DNA oligonucleotide was ligated to 3-Azido-7-hydroxycoumarin in the presence of BTT₍₁₎ DNA ligand or BTT₍₂₎-DNA ligand and sodium ascorbate. Error bars represent the standard deviation of three biological replicates. FIG. 1D depicts the structure of BTT_{(1,2)}-DNA ligand based on inductively coupled plasma mass spectrometry (ICP-MS). FIG. 1E depicts the structure of the linker connecting BTT to DNA in the BTT_{(1,2)}-DNA ligand.

FIGS. 2A-2I. Reaction kinetics of CuAAC accelerated by the BTT-DNA ligand with two different azido fluorogenic dyes. FIG. 2A depicts a strategy to synthesize BTT-DNA ligand via CuAAC using S1. The structure shown below the synthesis strategy depicts the linker that connects BTT to DNA in the BTT-DNA ligand. FIG. 2B is a schematic showing how an alkyne-labeled DNA oligonucleotide was ligated to a fluorogenic dye in the presence of BTT-DNA ligand and sodium ascorbate. FIG. 2C is a schematic showing how an alkyne-labeled DNA oligonucleotide was ligated to a fluorogenic dye in the presence of Cu²⁺, BTTAA, and sodium ascorbate. FIG. 2D depicts the structure of 3-Azido-7-hydroxycoumarin. FIG. 2E depicts the structure of CalFluor 488 azide. FIG. 2F shows the reaction kinetics of BTT-DNA ligand-assisted (left) or BTTAA ligand-assisted (right) CuAAC using 3-Azido-7-hydroxycoumarin. FIG. 2G shows the reaction rate of BTT-DNA ligand-assisted (black) or BTTAA ligand-assisted (red) CuAAC using 3-Azido-7-hydroxycoumarin. FIG. 2H shows the reaction kinetics of BTT-DNA ligand-assisted (left) or BTTAA ligand-assisted (right) CuAAC using CalFluor 488 azide. FIG. 2I shows the reaction rate of BTT-DNA ligand-assisted (black) or BTTAA ligand-assisted (red) CuAAC using CalFluor 488 azide. Each condition had three biological replicates.

FIGS. 3A-3C. Characterization of BTT-DNA ligand. FIG. 3A. The ligand was characterized using 15% TBE-Urea gel electrophoresis. Ladder (leftmost lane) bands are labeled in base pairs (bp). FIG. 3B. Results from a fluorogenic plate-reader assay show the ligation of 3-Azido-7-hydroxycoumarin using BTT-DNA at 1 hour. FIG. 3C. Yield of CuAAC reaction assisted by BTT-DNA and BTTAA using 3-Azido-7-hydroxycoumarin. In the CuAAC reaction assisted by BTTAA, 1 μM of M20-left-alkyne was ligated to the 7.5 μM of 3-Azido-7-hydroxycoumarin using 0.5 μM CuSO₄ and 1 μM BTTAA in the presence of 2.5 mM sodium ascorbate. In the CuAAC reaction assisted by BTT-DNA, 1 μM of M20-left-alkyne was ligated to the 7.5 μM of 3-Azido-7-hydroxycoumarin using 1 μM BTT-DNA in the presence of 2.5 mM sodium ascorbate. To get the yield, the measured fluorescence intensity of CuAAC reaction using 1 μM BTT-DNA or BTTAA was divided by that of CuAAC reaction using 50 μM CuSO₄ and 100 μM BTTAA which works as 100% reaction yield control. Data are presented as mean±standard deviation (SD). Error bars represent the standard deviation of three biological replicates.

FIGS. 4A-4F. Reaction kinetics of CuAAC accelerated by the BTT_{(1,2)}-DNA ligand with different azido fluorogenic dyes. FIG. 4A is a schematic showing how an alkyne-labeled DNA oligonucleotide was ligated to a fluorogenic dye in the presence of Cu²⁺, BTTAA, and sodium ascorbate (top) or in the presence of BTT_{(1,2)}-DNA ligand and sodium ascorbate (bottom). FIGS. 4B-4F show CuAAC reaction kinetics of the reactions shown in FIG. 4A. FIG. 4B shows the reaction kinetics with 3-Azido-7-hydroxycoumarin. FIG. 4C shows the reaction kinetics with CalFluor 647 azide. FIG. 4D shows the reaction kinetics with CalFluor 488 azide.

FIG. 4E shows the reaction kinetics with CalFluor 555 azide. FIG. 4F shows the reaction kinetics with CalFluor 580 azide. Error bars in FIGS. 4B-4F represent the standard deviation of a minimum of three biological replicates.

FIG. 5 shows optimization of the concentration of the 5′ alkyne DNA in CuAAC with commercial BTTAA ligand.

FIG. 6 shows optimization of the concentration of the 3-Azido-7-hydroxycoumarin in CuAAC with commercial BTTAA ligand.

FIG. 7 shows comparative reactivity of propargyl alkyne to DNA alkyne in CuAAC with commercial BTTAA ligand. Error bars represent the standard deviation of three biological replicates.

FIGS. 8A-8E BTT-DNA reaction kinetics. FIG. 8A shows reaction kinetics of CuAAC using 3-Azido-7-hydroxycoumarin with BTT-DNA that comprised a random DNA sequence. Error bars represent the standard deviation of three biological replicates. FIG. 8B. Results from a fluorogenic plate-reader assay of CuAAC using BTT-DNA ligand with different sequence lengths at 1 hour (bottom). FIG. 8C. Fluorogenic plate-reader assay of CuAAC using BTT-DNA ligand and the unmodified N₃-DNA probe to show the significance of BTT moiety (n=3). Comparison of the reactivity of CuAAC in the presence of BTT-DNA and unmodified N₃-DNA probe. In the CuAAC reaction, 1 μM of M20-left-alkyne was ligated to the 7.5 μM of 3-Azido-7-hydroxycoumarin using 1 μM of BTT-DNA or unmodified N₃-DNA probe in the presence of 2.5 mM sodium ascorbate. Error bars represent the standard deviation of three biological replicates. FIG. 8D. Comparison of DNA template-driven proximity ligation for BTT-DNA and unmodified N₃-DNA probe with spermine using 3-Azido-7-hydroxycoumarin. In the 2-hour CuAAC reaction, 1 M of M20-left-alkyne_20_1 was ligated to the 7.5 M of 3-Azido-7-hydroxycoumarin using 1 μM of BTT-DNA or unmodified N₃-DNA probe in the presence of 0.9 μM of DNA splint (DNAX1) and 100 μM of spermine and 1 M NaCl. FIG. 8E. Characterization of BTT-DNA ligand with different DNA lengths. Electrophoresis was run on a 15% TBE-Urea gel for 70 minutes at 180 volts.

FIG. 9. Stability of BTT-DNA ligand in accelerating CuAAC reaction over twelve months (n=2). In the CuAAC reaction, 1 μM of M20-left-alkyne was ligated to the 7.5 μM of 3-Azido-7-hydroxycoumarin using 1 μM of BTT-DNA in the presence of 2.5 mM sodium ascorbate.

FIGS. 10A-10C. DNA and RNA template-driven proximity ligation with BTT_{(1,2)}-DNA ligand. FIG. 10A is a schematic of nucleic acid template-driven proximity ligation. FIG. 10B shows fluorescence measurements of nucleic acid template-driven proximity ligation with DNA splints harboring different repeats of the binding sequence. Error bars represent the standard deviation of three biological replicates. FIG. 10C shows fluorescence microscopy of the intracellular 5-ethynyl uridine labeling of nascent RNAs via CuAAC in the presence of BTT_{(1,2)}-DNA ligand and BTTAA ligand in fixed cells. Fixed cells were treated with 1) CalFluor 647 azide and sodium ascorbate (leftmost panel); 2) CalFluor 647 azide, Cu²⁺, BTTAA, and sodium ascorbate (center left panel), 3) CalFluor 647 azide, BTT_{(1,2)}-DNA ligand (5 μM), and sodium ascorbate (center right panel); or 4) CalFluor 647 azide, BTT_{(1,2)}-DNA ligand (10 μM), and sodium ascorbate (right panel). CalFluor 647 azide labeling of nascent RNAs incorporated by 5-ethynyl uridine is shown in white; DAPI (4′,6-Diamidino-2-phenylindole dihydrochloride; CAS No: 28718-90-3) staining of nuclei is shown in blue.

FIGS. 11A-11C. Optimization of DNA and RNA template-driven proximity ligation with BTT_{(1,2)}-DNA ligand. FIG. 11A shows a comparison of DNA template-driven proximity ligation for 8 different sequences of 5′ alkyne DNA (SEQ ID NOs: 3-10) in the presence of spermine. FIG. 11B shows a comparison of DNA template-driven proximity ligation for 8 different 5′ alkyne DNA with or without spermine. Error bars represent the standard deviation of three biological replicates. These data are consistent with results observed with purified BTT-DNA (data not shown). FIG. 11C shows fluorescence measurements of DNA and RNA template-driven proximity ligation with a series of splint concentrations using the M20_alkyne_20_2 alkyne (SEQ ID NO:6). Error bars represent the standard deviation of three biological replicates.

FIGS. 12A-12F. DNA and RNA template-driven proximity ligation with BTT-DNA ligand. FIG. 12A is a schematic of nucleic acid template-driven proximity ligation. FIG. 12B shows the reaction kinetics of nucleic acid template-driven proximity ligation with DNA splints harboring different repeats of the binding sequence using 3-Azido-7-hydroxycoumarin and CalFluor 647 azide. Each condition had three biological replicates. FIG. 12C shows microscopy representative of individual RNA molecule detection with nucleic acid template-driven proximity ligation using BTT-DNA. Fixed cells were first treated with an alkyne-modified single-stranded DNA oligonucleotide probe, then treated with CalFluor 647 azide (10 μM), BTT-DNA ligand (20 μM), and sodium ascorbate. Individual RNA molecules are shown in white; DAPI staining of nuclei is shown in blue. FIG. 12D shows microscopy representative of individual RNA molecule detection with nucleic acid template-driven proximity ligation using BTT-DNA. The schematic diagram depicting the proximity click FISH of M20 transgene RNA in transgenic CHO-GFP-M20 cell line. Fixed cells were first treated with 0.2 μM alkyne-modified single-stranded DNA oligonucleotide probe (M20-left-alkyne_20_2; SEQ ID NO:6), then treated with CalFluor 647 azide (10 μM), BTT-DNA ligand (20 μM), and sodium ascorbate (2.5 mM) for 1 hour. Individual RNA molecules (white); GFP (green); DAPI staining of nuclei (blue). FIG. 12E shows photomicrographs of individual RNA molecule detection with nucleic acid template-driven proximity ligation using BTT-DNA with different CuAAC reaction times. Fixed cells were first treated with M20-left-alkyne_20_3t (0.2 μM; SEQ ID NO:9), then treated with CalFluor 647 azide (10 μM), BTT-DNA ligand (20 μM), and sodium ascorbate (2.5 mM) for 30 minutes, 1 hour and 2 hours. Individual RNA molecules (white); GFP (green); DAPI staining of nuclei (blue). FIG. 12F shows photomicrographs of individual RNA molecule detection with nucleic acid template-driven proximity ligation using BTT-DNA with eight different 5′ alkyne DNA. Fixed cells were first treated with 8 different 5′ alkyne DNA (0.2 μM; top row, SEQ ID NOs: 3-6, respectively; bottom row, SEQ ID NOs: 7-10, respectively), then treated with CalFluor 647 azide (10 μM), BTT-DNA ligand (20 μM), and sodium ascorbate (2.5 mM) for 1 hour. Individual RNA molecules (white); GFP (green); DAPI staining of nuclei (blue).

FIG. 13. shows a comparison of DNA template-driven proximity ligation for 8 different sequences 5′ alkyne DNA in the presence of spermine using CalFluor 488 azide.

FIG. 14 shows the reaction kinetics of DNA template-driven proximity ligation using BTT-DNA ligand with random DNA sequence. Each condition had two biological replicates.

FIG. 15 shows microscopy representative of individual RNA molecule detection with nucleic acid template-driven proximity ligation using BTT-DNA.

FIGS. 16A-16F. Detection and labeling of intracellular and extracellular biomolecules using CuAAC in the presence of BTT-DNA in/on fixed cells. Representative fluorescence microscopy (two biological replicates each) shows detection of nascent EdU-labeled DNA (FIG. 16A), nascent 5-EU-labeled RNA (FIG. 16B), nascent L-HPG-labeled protein (FIG. 16C), nascent Ac₄MaNAl-labeled sialic acid (FIG. 16D), and nascent propargyl choline-labeled phospholipids (FIG. 16E) via CuAAC in the presence of no ligand (top row), BTTAA (second row), or BTT-DNA ligand (third and fourth rows) in/on fixed cells. In the top row, fixed cells were treated with 10 μM CalFluor 647 azide and 2.5 mM sodium ascorbate; in the third and fourth rows, fixed cells were treated with 10 μM CalFluor 647 azide, 20 μM BTT-DNA ligand, and 2.5 mM sodium ascorbate. In the second row, fixed cells were treated with 10 μM CalFluor 647 azide, 10 μM CuSO₄, 20 μM BTTAA ligand, and 2.5 mM sodium ascorbate. CalFluor 647 azide labeling of nascent intracellular and extracellular biomolecules is shown in white; DAPI staining of nuclei is shown in blue. FIG. 16 F. The bar graphs quantify normalized signal for cells treated with BTT-DNA over the background (no ligand) for the fluorescence microscopy in FIGS. 16A-16E (quantification for BTTAA not shown).

FIGS. 17A-17B. Intracellular labeling and detection of nascent RNAs in fixed cells. FIG. 17A shows fluorescence microscopy (three biological replicates) of the intracellular 5-ethynyl uridine labeling of nascent RNAs via CuAAC in the presence of BTT_{(1,2)}-DNA ligand and BTTAA ligand in fixed cells. In the upper row, from left to right, fixed cells were treated with CalFluor 647 azide and sodium ascorbate (left); CalFluor 647 azide, BTT_{(1,2)}-DNA ligand (5 μM), and sodium ascorbate (middle); and CalFluor 647 azide, BTT_{(1,2)}-DNA ligand (10 μM), and sodium ascorbate (right). In the lower row, fixed cells were treated with CalFluor 647 azide, Cu²⁺, BTTAA, and sodium ascorbate. CalFluor 647 azide labeling of nascent RNAs incorporated by 5-ethynyl uridine is shown in white; DAPI staining of nuclei is shown in blue. FIG. 17B shows analysis of the fluorescent signal in the nucleus over the background in the cytoplasm for each condition. The dye only was the negative control. The fluorescent signal in the nucleus was divided by the background of the cytoplasm to get the signal-to-noise ratio for each condition. Then, the signal-to-noise ratio of each condition was normalized based on the negative control (i.e., by dividing by the signal from dye only). Error bars represent the standard deviation of fifteen replicates.

FIGS. 18A-18C. Intracellular labeling and detection of nascent RNAs in living cells. FIG. 18A is a schematic of metabolic 5-ethynyl uridine labeling of nascent RNAs and detection using a fluorogenic dye via CuAAC in the presence of BTT_{(1,2)}-DNA ligand in living cells. FIG. 18B shows analysis of the ratio of positive cells. Error bars represent the standard deviation of two biological replicates with a total six images. Left and right panels show different statistical comparisons. FIG. 18C shows representative fluorescence micrographs of live 5-ethynyl uridine-labeled cells subjected to CuAAC with a fluorogenic dye in the presence of BTT_{(1,2)}-DNA ligand or BTTAA ligand. CalFluor 647 azide labeling of nascent RNAs incorporated by 5-ethynyl uridine is shown in white; Hoechst 33342 (IUPAC name: 2-(4-ethoxyphenyl)-6-[6-(4-methylpiperazin-1-yl)-1H-1,3-benzodiazol-2-yl]-1H-1,3-benzodiazole trihydrochloride; CAS No. 875756-97-1) staining of nuclei is shown in blue.

FIG. 19. Intracellular labeling and detection of nascent RNAs in living cells. The figure shows representative fluorescence micrographs (three biological replicates) of live 5-ethynyl uridine-labeled cells subjected to CuAAC with a fluorogenic dye in the presence of BTT_{(1,2)}-DNA ligand and BTTAA ligand. In the upper row, from left to right, 5-ethynyl uridine-labeled live cells were treated with CalFluor 647 azide (left); CalFluor 647 azide, Cu²⁺, and BTTAA (middle); and CalFluor 647 azide and BTT_{(1,2)}-DNA ligand (10 M) (right). In the middle row, from left to right, 5-ethynyl uridine labeled live cells were treated with CalFluor 647 azide and sodium ascorbate (left); CalFluor 647 azide, Cu²⁺, BTTAA, and sodium ascorbate (middle); and CalFluor 647 azide, BTT_{(1,2)}-DNA ligand (10 μM), and sodium ascorbate (right). In the lower row, from left to right, live cells were treated with CalFluor 647 azide and sodium ascorbate (left); CalFluor 647 azide, Cu²⁺, BTTAA, and sodium ascorbate (middle); and CalFluor 647 azide, BTT_{(1,2)}-DNA ligand (10 μM), and sodium ascorbate (right). CalFluor 647 azide labeling of nascent RNAs incorporated by 5-ethynyl uridine is shown in white; Hoechst 33342 staining of nuclei is shown in blue.

FIGS. 20A-20B. Biocompatibility and performance of the BTT_{(1,2)}-DNA ligand assisted CuAAC on the surface of living cells. FIG. 20A shows a Jurkat cell growth assay that compared BTT_{(1,2)}-DNA ligand to commercial ligand. Error bars represent the standard deviation of four biological replicates. FIG. 20B shows metabolic labeling and detection of cell-surface sialic acids with BTT_{(1,2)}-DNA and commercial BTTAA ligand. CalFluor 647 azide labeling of sialic acids is shown in white; Hoechst 33342 staining of nuclei is shown in blue.

FIG. 21. Metabolic labeling and detection of cell-surface sialic acids with BTT_{(1,2)}-DNA and commercial BTTAA ligand (three biological replicates). In the upper row, from left to right, live cells were treated with CalFluor 647 azide and BTT_{(1,2)}-DNA ligand (10 μM) (left); and CalFluor 647 azide, Cu²⁺, and BTTAA (right). In the lower row, from left to right, live cells were treated with CalFluor 647 azide and sodium ascorbate (left); CalFluor 647 azide, BTT_{(1,2)}-DNA ligand (10 μM), and sodium ascorbate (middle); and CalFluor 647 azide, Cu²⁺, BTTAA, and sodium ascorbate (right). CalFluor 647 azide labeling of sialic acids is shown in white; Hoechst 33342 staining of nuclei is shown in blue.

FIGS. 22A-22C. Representative fluorescence microscopy (two biological replicates) of the cell-surface biomolecules labeling and detection in the presence of BTT-DNA on live cells. FIG. 22A shows metabolic labeling and detection of cell-surface sialic acids and Cho-containing phospholipid with BTT-DNA on live cells. In the left column, live cells were treated with 10 μM CalFluor 647 azide and 2.5 mM sodium ascorbate; in the middle column, live cells were treated with 10 μM CalFluor 647 azide, 20 M BTT-DNA ligand, and 2.5 mM sodium ascorbate. In the right column, live cells were treated with 10 μM CalFluor 647 azide, 10 μM CuSO₄, 20 μM BTTAA ligand, and 2.5 mM sodium ascorbate. CalFluor 647 azide labeling of sialic acids and Cho-containing phospholipid is shown in white; Hoechst 33342 staining of nuclei is shown in blue. FIG. 22B shows quantification of normalized cell-surface signal over nuclear signal for the fluorescence microscopy assisted by BTT-DNA in FIG. 22A. FIG. 22C shows quantification of normalized cell-surface signal over background for fluorescence microscopy in FIG. 22A.

FIGS. 23A-23B. Fluorescence microscopy of the cell viability of the cell-surface biomolecules labeling and detection in the presence of BTT-DNA on live cells. FIG. 23A shows fluorescence microscopy of the cell viability of the cell-surface sialic acid labeling and detection in the presence of BTT-DNA on live cells. FIG. 23B shows fluorescence microscopy of the cell viability of the cell-surface phospholipids labeling and detection in the presence of BTT-DNA on live cells.

FIGS. 24A-24C. FIG. 24A. Representative microscopy of cell reactive oxygen species with different sodium ascorbate concentrations on HeLa cells. Hoechst 33342 staining of nuclei of all cells (blue); CELLROX® Green Reagent staining of reactive oxygen species of nuclei of all cells (green). At bottom, representative fluorescence micrographs of live, 5-ethynyl uridine-labeled Jurkat cells subjected to CuAAC, after electroporation, with a fluorogenic dye in the presence of BTT-DNA ligand. CalFluor 647 azide labeling of nascent RNAs incorporated by 5-ethynyl uridine (white); Hoechst 33342 staining of nuclei (blue); SYTOX® green nucleic acid staining of nuclei of dead cells (green). FIG. 24B. Representative microscopy of cell viability in the presence of CuSO₄ and sodium ascorbate. Top row: Representative microscopy of cell viability assay with different sodium ascorbate treatment times on HeLa cells. Bottom row: Comparison of the effect of CuSO₄ and sodium ascorbate on cell viability. Hoechst 33342 staining of nuclei (blue); SYTOX® green nucleic acid staining of nuclei of dead cells (green). FIG. 24C. Photomicrographs of cell viability assay with different LIPOFECTAMINE® RNAIMAX® concentrations on HeLa cells. Hoechst 33342 staining of nuclei (blue); SYTOX® green nucleic acid staining of nuclei of dead cells (green).

FIG. 25. Photomicrographs of propargyl-choline treated live Hela cells in the presence of different CalFluor 647 azide concentrations. CalFluor 647 azide (red); Hoechst 33342 nuclei staining (blue).

FIG. 26. Photomicrographs of intracellular phospholipid labeling and detection in live cells in the presence of BTT-DNA with different transfection times. CalFluor 647 azide labeling of a choline-containing phospholipid (red); Hoechst 33342 nuclei staining (blue).

FIGS. 27A-27F. Biocompatibility of the BTT-DNA ligand (n=2). FIG. 27A. Representative microscopy of cell morphology (FIG. 27A), cell viability assay (FIG. 27B), cell reactive oxygen species (ROS) (FIG. 27C), cell superoxide (FIG. 27D), and cell apoptosis assay (FIG. 27E) comparing BTT-DNA ligand to copper on HeLa cells. Hoechst 33342 staining of nuclei of all cells (blue); SYTOX® green nucleic acid staining of nuclei of dead cells (green); CELLROX® Green Reagent of reactive oxygen species of nuclei of all cells (red), MITOSOX® green indicators of mitochondrial superoxide of all cells (yellow). FIG. 27F. Photomicrographs of biocompatibility of the BTT-DNA ligand to live cells cultured with propargyl choline.

FIGS. 28A-28F. Intracellular phospholipids labeling and detection in the presence of BTT-DNA in live cells. FIG. 28A. Workflow of metabolic labeling and detecting intracellular choline-containing phospholipid with BTT-DNA in live cells. Ox., oxidized copper. Red., reduced copper. FIG. 28B. Representative fluorescence microscopy (n=3) of the intracellular phospholipids labeling and detection in the presence of BTT-DNA in live cells. From top to bottom: live cells were treated with propargyl choline, LIPOFECTAMINE® RNAIMAX® transfection reagent, CalFluor 647 azide, and sodium ascorbate (top row); LIPOFECTAMINE® RNAIMAX® transfection reagent, BTT-DNA ligand, CalFluor 647 azide, and sodium ascorbate (second row); propargyl choline, LIPOFECTAMINE® RNAIMAX® transfection reagent, BTTAA, CuSO₄, CalFluor 647 azide, and sodium ascorbate (third row); or propargyl choline, LIPOFECTAMINE® RNAIMAX® transfection reagent, BTT-DNA ligand, CalFluor 647 azide, and sodium ascorbate (bottom row). CalFluor 647 azide labeling of choline-containing phospholipid (red); Hoechst 33342 nuclei staining (blue). FIG. 28C. Representative microscopy of chasing the intracellular phospholipids labeling in BTT-DNA presence for 2 hours. Live cells were treated with propargyl choline, LIPOFECTAMINE® RNAIMAX® transfection reagent, BTT-DNA ligand, CalFluor 647 azide, and sodium ascorbate. FIG. 28D. Analysis of the fluorescence enhancement for the fluorescence microscopy assisted by BTT-DNA in FIG. 28C. Error bars represent the standard deviation of 60 cells from three replicates. *p≤0.05, **p≤0.01, ***p≤0.001, and ****p≤0.0001. T-test was used. FIG. 28E. Representative fluorescence microscopy of the intracellular phospholipids labeling and detection in the presence of BTT-DNA and extracellular phospholipids labeling and detection in the presence of BTTAA-CuSO₄ complex in/on live cells. CalFluor 647 azide labeling of choline-containing phospholipid (red); Alexa Fluor 555 azide labeling of choline-containing phospholipid on the surface (yellow), Hoechst 33342 nuclei staining (blue). FIG. 28F. Representative fluorescence micrographs of the intracellular proteins labeling and detection in the presence of BTT-DNA in live cells. OPP stands for O-propargyl-puromycin. CalFluor 647 azide labeling of puromycin-containing proteins (red); Hoechst 33342 nuclei staining (blue).

FIG. 29. Photomicrographs of cell viability assay of intracellular phospholipids labeling and detection in the presence of BTT-DNA. Hoechst 33342 was used to stain nuclei of all cells (blue). SYTOX® green nucleic acid staining was used to stain nuclei of dead cells (green).

FIGS. 30A-30B. FIG. 30A. Schematic illustrating a method for intracellular labeling of proteins in live cells. FIG. 30B. Results showing intracellular labeling of proteins in live cells. CalFluor 647 azide labeling of OPP-labeled nascent proteins (red); Hoechst 33342 nuclei staining (blue).

FIGS. 31A-31B. FIG. 31A. Schematic illustrating a method for intracellular labeling of RNA nucleotides in live cells. FIG. 31B. Results showing intracellular labeling of RNA nucleotides in live cells. CalFluor 647 azide labeling of 5-EU (red); Hoechst 33342 nuclei staining (blue).

FIGS. 32A-32B. FIG. 32A. Schematic illustrating a method for intracellular labeling of nascent RNA in live cells. FIG. 32B. Results showing intracellular labeling of nascent RNA in live cells. CalFluor 647 azide labeling of 5-EU labeled nascent RNA (red); Hoechst 33342 nuclei staining (blue).

FIGS. 33A-33B. FIG. 33A. Intracellular phospholipid labeling and detection (two biological replicates) in the presence of BTT-DNA with different treatment temperature of sodium ascorbate in live cells. Live cells were treated with propargyl choline, LIPOFECTAMINE® RNAIMAX® transfection reagent, 30 M BTT-DNA ligand, and 80 μM CalFluor 647 azide. In the top row, cells were put on ice while adding 2 mM sodium ascorbate. In the bottom row, cells were in the 37° C. incubator while adding 2 mM sodium ascorbate. CalFluor 647 azide labeling of choline-containing phospholipid (red); Hoechst 33342 nuclei staining (blue). FIG. 33B. Time-lapse fluorescence microscopy (two biological replicates) of the intracellular phospholipids labeling with 30-minutes sodium ascorbate treatment on ice. Live cells were treated with propargyl choline, LIPOFECTAMINE® RNAIMAX® transfection reagent, 30 M BTT-DNA ligand, and 80 μM CalFluor 647 azide. Cells were put on ice while adding 2 mM sodium ascorbate.

### DETAILED DESCRIPTION

A description of example embodiments follows.

In some embodiments, this disclosure provides methods for copper (Cu(I))-catalyzed azide-alkyne cycloaddition (CuAAC), wherein the CuAAC may be performed in live cells (i.e., intracellularly) or on the surface of live cells. The methods and ligands disclosed herein are further described in the following, the contents of each of which is incorporated herein in its entirety:
U.S. Provisional Application No. 63/426,947;
U.S. Provisional Application No. 63/518,248;
International Application No. PCT/US2023/079299 (Publication No. WO 2024/112515);
U.S. application Ser. No. 19/131,506;
U.S. Provisional Application No. 63/664,711;
U.S. Provisional Application No. 63/768,874;
Keqing Nian, Yifang Liu, Laura Brigandi, Sara H. Rouhanifard. DNA-enhanced CuAAC ligand enables live-cell detection of intracellular biomolecules. bioRxiv 2022.11.10.515969; doi: https://doi.org/10.1101/2022.11.10.515969. Posted Nov. 10, 2022;
Keqing Nian, Yifang Liu, Laura Brigandi, Sara H. Rouhanifard. DNA-enhanced CuAAC ligand enables live-cell detection of intracellular biomolecules. bioRxiv 2022.11.10.515969; doi: https://doi.org/10.1101/2022.11.10.515969. Posted Jul. 31, 2024; and
Nian K, Liu Y, Qiu Y, Zhang Z, Brigandi L, Wanunu M, Rouhanifard SH. inCu-click: DNA-enhanced ligand enables live-cell, intracellular click chemistry reaction with copper catalyst. Nat Commun. 2025 May 23; 16 (1): 4788. doi: 10.1038/s41467-025-60143-3. PMID: 40410175; PMCID: PMC12102219.

As disclosed herein, a DNA oligomer-conjugated Cu(I)-catalyzed azide-alkyne cycloaddition (CuAAC)-accelerating ligand, e.g., BTT-DNA, is used for click reaction (e.g., ultra-sensitive click reaction) biomolecular labeling. The DNA oligomer attachment of the ligand serves several purposes, including (1) increasing availability of local copper atoms near the ligand, thus enabling the ligation of azide tags with much lower copper concentrations than commercially available CuAAC ligands; (2) allowing nucleic acid template-driven proximity ligation by designing the attached DNA sequence; (3) enabling the ligation of azide tags with up to 10-fold lower copper concentrations as compared to commercially available CuAAC ligands; and (4) allowing the CuAAC ligand and copper to traverse the cell and nuclear membrane. As described herein, embodiments of the methods enable the intracellular and cell surface labeling of biomolecules in live cells using fluorogenic dyes.

Bioorthogonal chemistry is a broadly applied conjugation strategy to study biological processes with molecular details1-4. These rapid reactions are highly selective without side reactions towards native functional groups5. One of the first bioorthogonal reactions reported was Cu(I)-catalyzed azide-alkyne cycloaddition (CuAAC), whereby a dipolar cycloaddition between a chemically inert terminal alkyne and an azide was facilitated6-9. The CuAAC reaction is compatible with aqueous conditions 10, is usually free of by-products1, and benefits from the commercial availability of a wide range of conjugates. However, the high copper catalyst concentration required in combination with ascorbate and oxygen can result in reactive oxygen species (ROS) that can be detrimental to nucleic acids11 and toxic to biological systems 12, thus limiting the CuAAC reaction inside live cells.

Biocompatible ligands have been developed to reduce the toxicity of the copper catalyst and enhance the reaction kinetics8,9, and copper-chelating azides have reduced the total copper requirement for the reaction 13. However, even with these developments, only some studies have achieved intracellular labeling using CuAAC. This is due to copper toxicity at the necessary concentration range, low uptake of reagents, and competing intracellular ligands capable of sequestering the copper. For example, a copper-chelating azide was used to enhance CuAAC reactivity 14 to detect an alkyne-labeled drug; however, this strategy precludes the use of fluorogenic azides. In another example, the CuAAC-accelerating ligand was conjugated to a cell-penetrating peptide and demonstrated to detect 1-homopropargylglycine using a fluorogenic dye15. However, the cell viability was diminished compared to commercial ligands due to the copper toxicity and yields for cytosolic proteins were significantly decreased. Therefore, CuAAC in live cells is typically used to label cell-surface biomolecules such as glycans8 and lipids 16. Alternatively, incorporating azide and alkyne reporters is frequently achieved in live cells. However, cell fixation is performed before CuAAC-driven ligation of detection probes 17,18.

Copper-free, strain-promoted azide-alkyne cycloaddition (SPAAC) may also be used for covalent tagging of biomolecules 19. This method is highly selective20,21 and may be used for intracellular labeling of biomolecules22. However, SPAAC is limited by the commercial availability of cyclooctynes23, providing an alkyne functional group for the reaction, reagent instability, and nucleophilic addition with cellular nucleophiles20. Thus, developing a CuAAC reaction that may be used for intracellular, live-cell labeling is desirable.

In some embodiments, the disclosed methods utilize DNA oligomer-conjugated CuAAC-accelerating ligands, BTT_{(1,2)}-DNA and BTT-DNA, that can reduce the required copper concentration while enhancing the reaction kinetics of CuAAC. As shown herein, the DNA oligomer attachment increased the availability of local copper atoms in proximity to the ligand, thus enabling the ligation of azide tags with much lower copper concentrations than commercially available CuAAC ligands. The DNA oligomer attachment also enabled a nucleic acid template-driven proximity ligation by designing the attached DNA sequence. Further, as shown herein, BTT_{(1,2)}-DNA and BTT-DNA enabled the ligation of azide with up to 10-fold and 5-fold lower ligand concentrations, respectively, in the fluorogenic plate-reader assay with various fluorogenic azide probes24. In addition, as shown herein, increasing the number of binding sites of the nucleic acid template increased the CuAAC reaction yield accelerated by BTT_{(1,2)}-DNA or BTT-DNA. Moreover, as shown herein, BTT_{(1,2)}-DNA and BTT-DNA enhanced the intracellular 5-ethynyl uridine detection of nascent RNAs with much lower ligand concentration in fixed cells (e.g., a 12-fold lower ligand concentration with BTT_{(1,2)}-DNA). BTT-DNA enabled the ligation of fluorogenic dyes on the surface of live cells to metabolically labeled sialylated glycans and Cho-containing phospholipids with a lower ligand concentration; BTT_{(1,2)}-DNA was similarly demonstrated herein to enable the live-cell labeling of sialylated glycans with a 4-fold lower ligand concentration. Additionally, as shown herein, individual RNA molecules in cells were detected using the new DNA oligomer-conjugated CuAAC ligands BTT_{(1,2)}-DNA and BTT-DNA; BTT_{(1,2)}-DNA was further demonstrated to enable live-cell labeling and specific detection of nascent RNA molecules in the nuclear compartment of cells. Further, proximity ligation was applied to detect individual RNA molecules in the cell nucleus and cytoplasm using click RNA fluorescent in situ hybridization (FISH) assisted by the DNA oligomer-conjugated CuAAC ligand in fixed cells. DNA also provided a convenient method for intracellular delivery to encapsulate the copper within a liposome. It was demonstrated that BTT-DNA could protect the live cells from the toxicity and cellular perturbation caused by Cu(I) and sodium ascorbate. Additionally, BTT-DNA was shown to enable the live-cell, intracellular labeling of nascent phospholipids and proteins. In short, BTT_{(1,2)}-DNA and BTT-DNA enabled sensitive detection of biomolecules in fixed and live cells, advancing efforts toward applying CuAAC for intracellular, live-cell applications.

As disclosed herein, DNA-conjugated CuAAC-accelerating ligands, BTT_{(1,2)}-DNA and BTT-DNA, were developed. These ligands reduced the required copper concentration for CuAAC while maintaining fast reaction kinetics. With these new CuAAC-accelerating ligands, the total ligand concentration required for the CuAAC in vitro may be decreased to the nanomolar range while remaining complexed to an average of 7.5 (BTT_{(1,2)}-DNA) or 10 (BTT-DNA) copper molecules per ligand with different fluorogenic azido dyes, thus eliminating the need for additional copper in the reaction. These new ligands were also shown to significantly drive CuAAC, enable the CuAAC reagents to traverse the cell and nuclear membranes, and exhibit stronger intracellular labeling in fixed cells and achieving live cell detection of biomolecules in the nucleus.

Previous studies have shown DNA splint-enhanced CuAAC²⁶⁻²⁸ by bringing together the azide and the alkyne probes; however, these systems preclude the use of fluorogenic dyes that are ideal for live-cell applications. As shown herein, the BTT_{(1,2)}-DNA and BTT-DNA ligands were able to drive further the CuAAC reaction kinetics when hybridized to a DNA or RNA splint close to an alkyne-modified probe and clicked to a fluorogenic azide. This demonstrated that a completely non-fluorescent system may be used to drive the CuAAC reaction kinetics of a fluorogenic dye. As demonstrated herein, the reaction was enhanced with the addition of spermine (with NaCl for BTT-DNA experiments; without NaCl for BTT_{(1,2)}-DNA experiments) to the mixture before CuAAC. Without being bound by any theory, the spermine may have contributed to the stability of the DNA-DNA duplex and DNA-RNA duplex²⁹, which was useful for the subsequent CuAAC reaction. Additionally, without being bound by any theory, spermine may have helped mask the negative charge of oligonucleotides³⁰ that may have sequestered the copper away from the desired ligation site. As shown herein, DNA splints with multiple binding sites amplified the total fluorescence produced. Further, the BTT-DNA ligand is shown herein to enhance the RNA template-driven proximity ligation in situ in fixed cells.

The experiments disclosed herein demonstrated that the BTT-DNA ligand can enhance the RNA template-driven proximity ligation in situ in fixed cells. The sensitive and specific detection of individual RNA molecules in the nucleus as well as the cytoplasm was achieved, advancing the effort toward CuAAC application in detection and tracking single molecule RNA in live cells without a wash step.

The experiments disclosed herein demonstrated that the BTT_{(1,2)}-DNA and BTT-DNA ligands can be used for standard CuAAC ligation assays, such as 5-ethynyl uridine labeling and detection of nascent RNAs in fixed cells. The BTT_{(1,2)}-DNA and BTT-DNA ligands outperformed the commercial reagent⁹ with lower ligand concentration in fixed cells (e.g., a 12-fold lower ligand concentration in fixed cells with BTT_{(1,2)}-DNA). Additionally, the fluorescence produced in the nucleus was much higher for the reaction in the presence of 10 μM BTT_{(1,2)}-DNA ligand. The results of the experiments disclosed herein indicated that the BTT_{(1,2)}-DNA and BTT-DNA ligands can also outperform the commercial ligand in biological environments with lower concentration and higher yield.

As disclosed herein, BTT_{(1,2)}-DNA ligand was demonstrated to lower the cytotoxicity induced by copper for at least 12 hours. Interestingly, the 5 μM BTT_{(1,2)}-DNA ligand concentration offered the greatest protection from copper toxicity until 6 hours, after which the 10 μM BTT_{(1,2)}-DNA ligand concentration offered the greatest protection. This observation was made across multiple biological replicates. Without being bound by any theory, the prolonged protection may be because the copper molecules are not covalently bound to the ligand, and therefore will dissociate as they achieve equilibrium with the environment; the equilibrium takes longer for the BTT_{(1,2)}-DNA ligand at high concentration, leading to longer-term protection at the 10 μM BTT_{(1,2)}-DNA ligand concentration.

The BTT_{(1,2)}-DNA ligand displayed some unique properties when used for live cell labeling. As disclosed herein, the ligation of fluorogenic dyes on the surface of live cells to metabolically labeled sialylated glycans was achieved with a 4-fold lower ligand concentration than with the commercial ligand at low temperature. Interestingly, when the CuAAC reaction temperature was increased from 0° C. to 37° C., the fluorescent signal produced on the cell membrane diminished for reactions using the BTT_{(1,2)}-DNA ligand, and several cells showed high fluorescent signal in the nucleus, which did not appear in the commercial BTTAA ligand. Without being bound by any theory, because this reaction was performed with a fluorogenic dye, this result suggested that 1) the BTT_{(1,2)}-DNA ligand was freely entering the cells; and 2) Ac₄ManNAl may have been visualized as it was getting metabolized into CMP-Neu5N-(4pentynoyl) neuraminic acid (CMPSiaNAl) in the nucleus²². The primary reason why DNA cannot freely diffuse into cells is that the negative charge of the DNA is repelled by the negative charge on the surface of mammalian cells. Without being bound by any theory, it is possible that the pre-complexed copper molecules associated with the BTT_{(1,2)}-DNA ligand were sufficiently masking the charge of the DNA, thus allowing the ligand to diffuse freely.

As disclosed herein, the BTT-DNA ligand can also be used for live cell labeling. The ligation of fluorogenic dyes on the surface of live cells to metabolically labeled sialylated glycans and Cho-containing phospholipids occurred in the presence of BTT-DNA with lower ligand concentration than with the commercial ligand at low temperature.

The CuAAC-accelerating ligands, BTT_{(1,2)}-DNA and BTT-DNA, can drive an intercellular CuAAC reaction. The BTT_{(1,2)}-DNA ligand was demonstrated herein to be useful for intercellular detection of EU-labeled nascent RNAs and cell surface detection of sialic acids in live cells. This reaction is typically performed by metabolically labeling live cells with EU, fixing and permeabilizing the cells, and then detecting the EU labeled RNA using CuAAC; however, BTT_{(1,2)}-DNA enabled detection in live cells. Further development led to the production of the BTT-DNA ligand, which was demonstrated herein to be useful for intracellular detection of RNA, protein, and phospholipids, as well as cell surface detection of sialic acid and phospholipids, in live cells. BTT-DNA was also demonstrated herein to be useful for intracellular detection of DNA in fixed cells, and could be used to detect DNA in live cells. Overall, the methods herein disclose use of CuAAC-accelerating ligands for sensitive detection of intracellular and cell surface biomolecules in fixed and live cells.

### EMBODIMENTS

In one aspect, the disclosure herein provides a Cu(I)-catalyzed azide-alkyne cycloaddition (CuAAC)-accelerating ligand comprising a polynucleotide.

In some embodiments, the CuAAC-accelerating ligand further comprises one or more copper chelators and a linker connecting the polynucleotide to each of the copper chelators.

In some embodiments, the polynucleotide: comprises a single-stranded DNA, RNA, XNA, modified polynucleotide, an aptamer, or a combination thereof; is complementary to one or more other polynucleotides, which comprises a template, a target, a payload, or a combination thereof; is conjugated at a 3′ end, a 5′ end, and/or an internal base to a linker; binds a polypeptide; is conjugated to at least one of: a nanoparticle, a small molecule, a liposome, an antibody or antigen-binding fragment thereof, a detectable molecule, or a combination thereof; or any combination of the foregoing.

In some embodiments, the CuAAC-accelerating ligand is immobilized on the surface of a nanoparticle; chelates about 7 to 10 copper atoms; or both of the foregoing.

In some embodiments, the CuAAC-accelerating ligand further comprises: one or two copper chelators; a nanoparticle, a small molecule, a liposome, an antibody or antigen-binding fragment thereof, a detectable molecule, a chemical functional group, or a combination thereof; or any combination of the foregoing.

In some embodiments, the one or more copper chelators comprise 2-(4-((Bis((1-(tert-butyl)-1H-1,2,3-triazol-4-yl)methyl)amino)methyl)-1H-1,2,3-triazol-1-yl) (BTT).

In another aspect, the disclosure herein provides a method of synthesizing a Cu(I)-catalyzed azide-alkyne cycloaddition (CuAAC)-accelerating ligand comprising a polynucleotide, wherein the method comprises the following steps in order:
a) preparing a CuAAC reaction mixture comprising:
   i. an azide-labeled polynucleotide;
   ii. a copper chelator, wherein the copper chelator comprises an alkyne; and
   iii. a copper source;
b) adding sodium ascorbate to the CuAAC reaction mixture, thereby producing a sodium ascorbate-CuAAC reaction mixture; and
c) shaking the sodium ascorbate-CuAAC reaction mixture;
   a) thereby driving Cu(I)-catalyzed azide-alkyne cycloaddition of the azide-labeled polynucleotide to the copper chelator to produce the CuAAC-accelerating ligand comprising a polynucleotide.

In some embodiments, the azide-labeled polynucleotide comprises one or two azide groups; the copper chelator is biocompatible; the copper source is copper sulfate; the CuAAC reaction mixture further comprises a CuAAC-accelerating ligand; the shaking is performed at about 600 rpm, at about 0° C. to 37° C., or both; the method further comprises purifying the CuAAC-accelerating ligand comprising a polynucleotide from a remainder of the sodium ascorbate-CuAAC reaction mixture; or any combination of the foregoing.

In some embodiments, the sodium ascorbate-CuAAC reaction mixture comprises: copper sulfate at a molar concentration about 13-fold greater than the molar concentration of the azide-labeled polynucleotide; the CuAAC-accelerating ligand at a molar concentration about 25-fold greater than the molar concentration of the azide-labeled polynucleotide; sodium ascorbate at a molar concentration about 417-fold greater than the molar concentration of the azide-labeled polynucleotide; or any combination of the foregoing.

In some embodiments, the copper chelator comprises N,N-bis((1-tert-butyl-1H-1,2,3-triazol-4-yl)methyl) prop-2-yn-1-amine (S1 alkyne); the CuAAC-accelerating ligand comprises 2-(4-((Bis((1-(tert-butyl)-1H-1,2,3-triazol-4-yl)methyl)amino)methyl)-1H-1,2,3-triazol-1-yl) acetic acid (BTTAA); the purifying is performed with a 3.5 kD molecular weight threshold; or any combination of the forgoing.

In some embodiments, the azide-labeled polynucleotide comprises: an azide-labeled polynucleotide that comprises one azide group; and an azide-labeled polynucleotide that comprises two azide groups.

In some embodiments, the CuAAC reaction mixture comprises a copper chelator at a molar concentration about 100-fold greater than the molar concentration of the azide-labeled polynucleotide; the shaking is performed for about 60 minutes; or both of the foregoing.

In some embodiments, the CuAAC reaction mixture comprises about 6 μM azide-labeled polynucleotide, about 600 μM S1 alkyne, about 75 μM copper sulfate, about 150 μM BTTAA, or a combination of any of the foregoing; the sodium ascorbate-CuAAC reaction mixture comprises about 2.5 mM sodium ascorbate; or any combination of the foregoing.

In some embodiments, the azide-labeled polynucleotide comprises one azide group.

In some embodiments, the CuAAC reaction mixture comprises a copper chelator at a molar concentration about 200-fold greater than the molar concentration of the azide-labeled polynucleotide; the shaking is performed for about 30 minutes; or both of the foregoing.

In some embodiments, the CuAAC reaction mixture comprises about 6 μM azide-labeled polynucleotide, about 1200 μM S1 alkyne, about 75 μM copper sulfate, about 150 μM BTTAA, or any combination of the foregoing; the sodium ascorbate-CuAAC reaction mixture comprises about 2.5 mM sodium ascorbate; or any combination of the foregoing.

In yet another aspect, the disclosure herein provides a method of ligating a payload to a target, the method comprising performing a Cu(I)-catalyzed azide-alkyne cycloaddition (CuAAC) in the presence of a CuAAC-accelerating ligand comprising a polynucleotide.

In some embodiments, the CuAAC occurs at a rate of at least about 1×10⁻⁵ min⁻¹ nM⁻¹.

In some embodiments, the CuAAC occurs: inside or on the surface of a live cell, a fixed cell, a dead cell, or a combination thereof; inside or on the surface of a population of cells, an organoid, or a tissue comprising a live cell, a fixed cell, a dead cell, or a combination thereof; in a living subject; or any combination of the foregoing.

In some embodiments, the CuAAC occurs inside a live cell.

In some embodiments, the target comprises: an alkyne-derivatized thymidine analog, an alkyne-derivatized uridine analog, an alkyne-derivatized methionine analog, an alkyne-derivatized monosaccharide, an alkyne-derivatized choline, a library of alkyne-containing compounds, or any combination of the foregoing.

In some embodiments, the target comprises: 5-ethynyl-2′-deoxyuridine (EdU), 5-ethynyl uridine (EU), L-homopropargyl (L-HPG), N-(4-pentynoyl) mannosamine (Ac₄MaNAl), propargyl choline, or any combination of the foregoing.

In some embodiments, performing the CuAAC comprises the steps of:
a) contacting a cell, a population of cells, an organoid, or a tissue with the target, wherein the target comprises an alkyne-containing compound;
b) further contacting the cell, the population of cells, the organoid, or the tissue with the payload and the ligand, wherein the payload comprises a detectable molecule comprising an azide, thereby producing a compound ligated to a detectable molecule; and
c) detecting the detectable molecule, its location, or both the detectable molecule and its location.

In some embodiments, the detectable molecule comprises a fluorogenic azide.

In some embodiments, the compound comprises a small molecule or a drug, or a library of compounds comprising small molecules or drugs.

In some embodiments, the method further comprises performing the CuAAC in the presence of a nucleic acid template, wherein the template or a portion thereof is complementary to: the polynucleotide; and the payload and/or the target.

In some embodiments, the method further comprises performing the CuAAC in the presence of spermine.

In yet another aspect, the disclosure herein provides a method of labeling a live cell, the method comprising performing Cu(I)-catalyzed azide-alkyne cycloaddition (CuAAC) of a label to a target in the presence of a CuAAC-accelerating ligand comprising a polynucleotide in a live cell.

In some embodiments, the labeling is intracellular labeling.

In some embodiments, the target is an RNA comprising an alkyne-derivatized uridine analog.

In some embodiments, the target is a DNA comprising an alkyne-derivatized thymidine analog.

Example embodiments are set forth below.

1. A Cu(I)-catalyzed azide-alkyne cycloaddition (CuAAC)-accelerating ligand comprising a polynucleotide.

2. The CuAAC-accelerating ligand of embodiment 1, wherein the ligand further comprises:
a) one or more copper chelators; and
b) a linker connecting the polynucleotide to each of the copper chelators.

3. The CuAAC-accelerating ligand of embodiment 1 or 2, wherein the polynucleotide:
a) comprises a single-stranded DNA, RNA, XNA, modified polynucleotide, an aptamer, or a combination thereof;
b) is complementary to one or more other polynucleotides, which comprises a template, a target, a payload, or a combination thereof;
c) is conjugated at a 3′ end, a 5′ end, and/or an internal base to a linker;
d) binds a polypeptide;
e) is conjugated to at least one of: a nanoparticle, a small molecule, a liposome, an antibody or antigen-binding fragment thereof, a detectable molecule, or a combination thereof; or
f) any combination of the foregoing.

4. The CuAAC-accelerating ligand of any one of embodiments 1-3, wherein the ligand:
a) is immobilized on the surface of a nanoparticle;
b) chelates about 7 to 10 copper atoms; or
c) both of the foregoing.

5. The CuAAC-accelerating ligand of any one of embodiments 1-4, wherein the ligand further comprises:
a) one or two copper chelators;
b) a nanoparticle, a small molecule, a liposome, an antibody or antigen-binding fragment thereof, a detectable molecule, a chemical functional group, or a combination thereof; or
c) any combination of the foregoing.

6. The CuAAC-accelerating ligand of embodiment 2 or 5, wherein the one or more copper chelators comprise 2-(4-((Bis((1-(tert-butyl)-1H-1,2,3-triazol-4-yl)methyl)amino)methyl)-1H-1,2,3-triazol-1-yl) (BTT).

7. A method of synthesizing a Cu(I)-catalyzed azide-alkyne cycloaddition (CuAAC)-accelerating ligand comprising a polynucleotide, wherein the method comprises the following steps in order:
a) preparing a CuAAC reaction mixture comprising:
   i) an azide-labeled polynucleotide;
   ii) a copper chelator, wherein the copper chelator comprises an alkyne; and
   iii) a copper source;
b) adding sodium ascorbate to the CuAAC reaction mixture, thereby producing a sodium ascorbate-CuAAC reaction mixture; and
c) shaking the sodium ascorbate-CuAAC reaction mixture; thereby driving Cu(I)-catalyzed azide-alkyne cycloaddition of the azide-labeled polynucleotide to the copper chelator to produce the CuAAC-accelerating ligand comprising a polynucleotide.

8. The method of embodiment 7, wherein:
a) the azide-labeled polynucleotide comprises one or two azide groups;
b) the copper chelator is biocompatible;
c) the copper source is copper sulfate;
d) the CuAAC reaction mixture further comprises a CuAAC-accelerating ligand;
e) the shaking is performed at about 600 rpm, at about 0° C. to 37° C., or both;
f) the method further comprises purifying the CuAAC-accelerating ligand comprising a polynucleotide from a remainder of the sodium ascorbate-CuAAC reaction mixture;
g) or any combination of the foregoing.

9. The method of embodiment 8, wherein the sodium ascorbate-CuAAC reaction mixture comprises:
a) copper sulfate at a molar concentration about 13-fold greater than the molar concentration of the azide-labeled polynucleotide;
b) the CuAAC-accelerating ligand at a molar concentration about 25-fold greater than the molar concentration of the azide-labeled polynucleotide;
c) sodium ascorbate at a molar concentration about 417-fold greater than the molar concentration of the azide-labeled polynucleotide; or
d) any combination of the foregoing.

10. The method of embodiment 8 or 9, wherein:
a) the copper chelator comprises N,N-bis((1-tert-butyl-1H-1,2,3-triazol-4-yl)methyl) prop-2-yn-1-amine (S1 alkyne);
b) the CuAAC-accelerating ligand comprises 2-(4-((Bis((1-(tert-butyl)-1H-1,2,3-triazol-4-yl)methyl)amino)methyl)-1H-1,2,3-triazol-1-yl) acetic acid (BTTAA);
c) the purifying is performed with a 3.5 kD molecular weight threshold; or
d) any combination of the forgoing.

11. The method of any one of embodiments 7-10, wherein the azide-labeled polynucleotide comprises:
a) an azide-labeled polynucleotide that comprises one azide group; and
b) an azide-labeled polynucleotide that comprises two azide groups.

12. The method of any one of embodiments 7-11, wherein:
a) the CuAAC reaction mixture comprises a copper chelator at a molar concentration about 100-fold greater than the molar concentration of the azide-labeled polynucleotide;
b) the shaking is performed for about 60 minutes; or
c) both of the foregoing.

13. The method of any one of embodiments 7-12, wherein:
a) the CuAAC reaction mixture comprises:
   i) about 6 μM azide-labeled polynucleotide;
   ii) about 600 μM S1 alkyne;
   iii) about 75 μM copper sulfate;
   iv) about 150 μM BTTAA;
   v) or a combination of any of the foregoing;
b) the sodium ascorbate-CuAAC reaction mixture comprises about 2.5 mM sodium ascorbate; or
c) any combination of the foregoing.

14. The method of any one of embodiments 7-12, wherein the azide-labeled polynucleotide comprises one azide group.

15. he method of any one of embodiments 7-10 or 14, wherein:
a) the CuAAC reaction mixture comprises a copper chelator at a molar concentration about 200-fold greater than the molar concentration of the azide-labeled polynucleotide;
b) the shaking is performed for about 30 minutes; or
c) both of the foregoing.

16. The method of any one of embodiments 7-10 or 14-15, wherein:
a) the CuAAC reaction mixture comprises:
   i) about 6 μM azide-labeled polynucleotide;
   ii) about 1200 μM S1 alkyne;
   iii) about 75 M copper sulfate;
   iv) about 150 μM BTTAA; or
   v) any combination of the foregoing;
b) the sodium ascorbate-CuAAC reaction mixture comprises about 2.5 mM sodium ascorbate; or
c) any combination of the foregoing.

17. A method of ligating a payload to a target, the method comprising performing a Cu(I)-catalyzed azide-alkyne cycloaddition (CuAAC) in the presence of a CuAAC-accelerating ligand comprising a polynucleotide.

18. The method of embodiment 17, wherein the CuAAC occurs at a rate of at least about 1×10⁻⁵ min⁻¹ nM⁻¹.

19. The method of embodiment 17 or 18, wherein the CuAAC occurs:
a) inside or on the surface of a live cell, a fixed cell, a dead cell, or a combination thereof;
b) inside or on the surface of a population of cells, an organoid, or a tissue comprising a live cell, a fixed cell, a dead cell, or a combination thereof;
c) in a living subject; or
d) any combination of the foregoing.

20. The method of any one of embodiments 17-19, wherein the CuAAC occurs inside a live cell.

21. The method of any one of embodiments 17-20, wherein the target comprises:
a) an alkyne-derivatized thymidine analog;
b) an alkyne-derivatized uridine analog;
c) an alkyne-derivatized methionine analog;
d) an alkyne-derivatized monosaccharide;
e) an alkyne-derivatized choline;
f) a library of alkyne-containing compounds; or
g) any combination of the foregoing.

22. The method of any one of embodiments 17-21, wherein the target comprises:
a) 5-ethynyl-2′-deoxyuridine (EdU);
b) 5-ethynyl uridine (EU);
c) L-homopropargyl (L-HPG);
d) N-(4-pentynoyl) mannosamine (Ac₄MaNAl);
e) propargyl choline; or
f) any combination of the foregoing.

23. The method of any one of embodiments 17-20, wherein performing the CuAAC comprises the steps of:
a) contacting a cell, a population of cells, an organoid, or a tissue with the target, wherein the target comprises an alkyne-containing compound;
b) further contacting the cell, the population of cells, the organoid, or the tissue with the payload and the ligand, wherein the payload comprises a detectable molecule comprising an azide, thereby producing a compound ligated to a detectable molecule; and
c) detecting the detectable molecule, its location, or both the detectable molecule and its location.

24. The method of embodiment 23, wherein the detectable molecule comprises a fluorogenic azide.

25. The method of embodiment 23, wherein the compound comprises a small molecule or a drug, or a library of compounds comprising small molecules or drugs.

26. The method of any one of embodiments 17-25, the method further comprising performing the CuAAC in the presence of a nucleic acid template, wherein the template or a portion thereof is complementary to:
a) the polynucleotide; and
b) the payload and/or the target.

27. The method of embodiment 26, wherein the method further comprising performing the CuAAC in the presence of spermine.

28. A method of labeling a live cell, the method comprising performing Cu(I)-catalyzed azide-alkyne cycloaddition (CuAAC) of a label to a target in the presence of a CuAAC-accelerating ligand comprising a polynucleotide in a live cell.

29. The method of embodiment 28, wherein the labeling is intracellular labeling.

30. The method of embodiment 28 or 29, wherein the target is an RNA comprising an alkyne-derivatized uridine analog.

31. The method of embodiment 28 or 29, wherein the target is a DNA comprising an alkyne-derivatized thymidine analog.

### Compositions

In one aspect, the disclosure herein provides a Cu(I)-catalyzed azide-alkyne cycloaddition (CuAAC)-accelerating ligand comprising a polynucleotide (i.e., a CuAAC-accelerating ligand, wherein the ligand comprises a polynucleotide).

In some embodiments, the CuAAC-accelerating ligand comprising a polynucleotide comprises BTT_{(1,2)}-DNA. In some embodiments, BTT_{(1,2)}-DNA comprises a mixture of BTT₍₁₎-DNA and BTT₍₂₎-DNA.

In some embodiments, the CuAAC-accelerating ligand comprising a polynucleotide comprises BTT-DNA.

In some embodiments, the CuAAC-accelerating ligand comprising a polynucleotide comprises BTT₍₁₎-DNA. In some embodiments, the CuAAC-accelerating ligand comprising a polynucleotide comprises BTT₍₂₎-DNA. In some embodiments, the CuAAC-accelerating ligand comprising a polynucleotide comprises BTT_{(1,2)}-DNA, BTT₍₁₎-DNA, BTT₍₂₎-DNA, BTT-DNA, or a combination thereof.

In some embodiments, the CuAAC-accelerating ligand comprising a polynucleotide comprises one or more copper chelators. In some embodiments, the CuAAC-accelerating ligand comprising a polynucleotide comprises one copper chelator. In some embodiments, the CuAAC-accelerating ligand comprising a polynucleotide comprises two copper chelators. In some embodiments, the CuAAC-accelerating ligand comprising a polynucleotide comprises more than two copper chelators. In some embodiments, the CuAAC-accelerating ligand comprising a polynucleotide comprises two or more of the same copper chelator. In some embodiments, the CuAAC-accelerating ligand comprising a polynucleotide comprises two or more different copper chelators.

In some embodiments, the CuAAC-accelerating ligand comprising a polynucleotide comprises a linker. In some embodiments, the CuAAC-accelerating ligand comprising a polynucleotide comprises two linkers. In some embodiments, the CuAAC-accelerating ligand comprising a polynucleotide comprises more than two linkers. In some embodiments, a linker connects the polynucleotide to each of the copper chelators. In some embodiments, a linker connects the polynucleotide to one copper chelators. In some embodiments, a linker connects the polynucleotide to each of two copper chelators. In some embodiments, a linker connects the polynucleotide to each of more than two copper chelators. In some embodiments, one linker connects the polynucleotide to one copper chelator. In some embodiments, two linkers connect the polynucleotide to two copper chelators. In some embodiments, more than two linkers connect the polynucleotide to more than two copper chelators. In some embodiments, a first linker connects the polynucleotide to a first copper chelator. In some embodiments, a second linker connects the polynucleotide to a second copper chelator. In some embodiments, a first linker connects the polynucleotide to a first copper chelator and a second linker connects the polynucleotide to a second copper chelator.

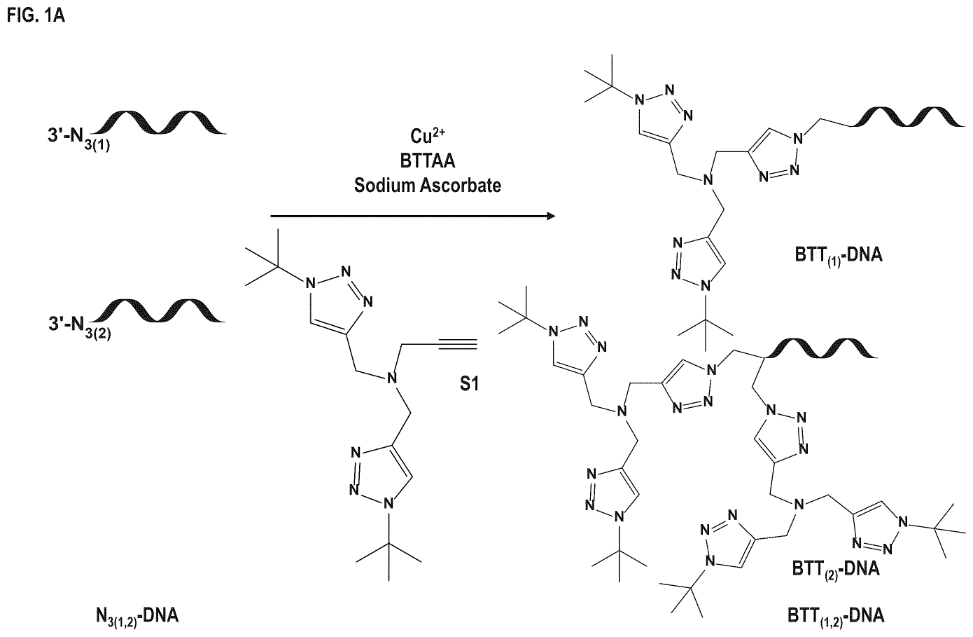

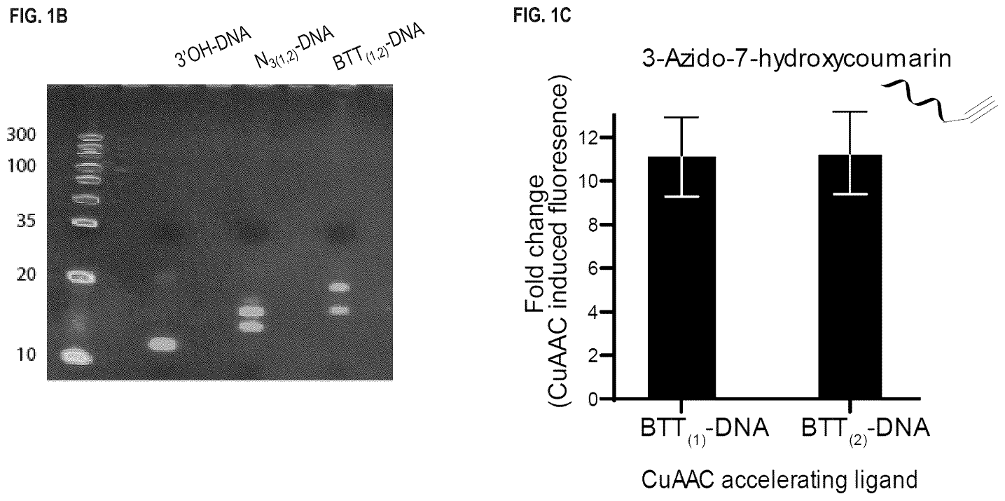

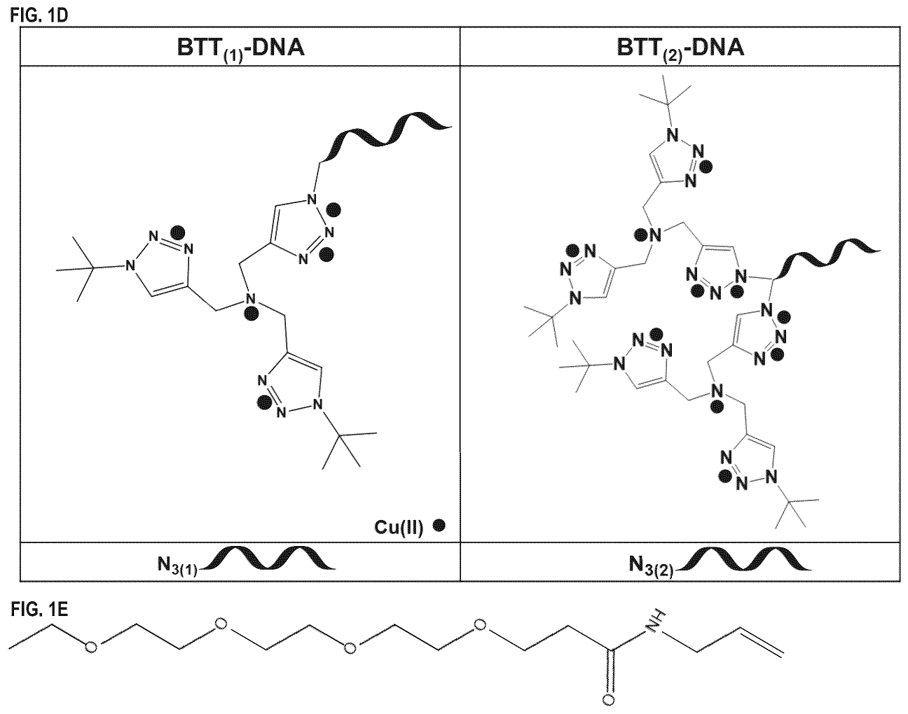

In some embodiments, a linker comprises the structure set forth in FIG. 1E. In some embodiments, BTT_{(1,2)}-DNA comprises the structure set forth in FIG. 1E. In some embodiments, BTT₍₁₎-DNA comprises the structure set forth in FIG. 1E. In some embodiments, BTT₍₂₎-DNA comprises the structure set forth in FIG. 1E. In some embodiments, BTT-DNA comprises the structure set forth in FIG. 1E. In some embodiments, BTT_{(1,2)}-DNA, BTT₍₁₎-DNA, BTT₍₂₎-DNA, BTT-DNA, or a combination thereof comprises the structure set forth in FIG. 1E. In some embodiments, a linker comprising the structure set forth in FIG. 1E is used in the synthesis of BTT_{(1,2)}-DNA. In some embodiments, a linker comprising the structure set forth in FIG. 1E is used in the synthesis of BTT)-DNA. In some embodiments, a linker comprising the structure set forth in FIG. 1E is used in the synthesis of BTT₍₂₎-DNA. In some embodiments, a linker comprising the structure set forth in FIG. 1E is used in the synthesis of BTT-DNA. In some embodiments, a linker comprising the structure set forth in FIG. 1E is used in the synthesis of BTT_{(1,2)}-DNA, BTT₍₁₎-DNA, BTT₍₂₎-DNA, BTT-DNA, or a combination thereof.

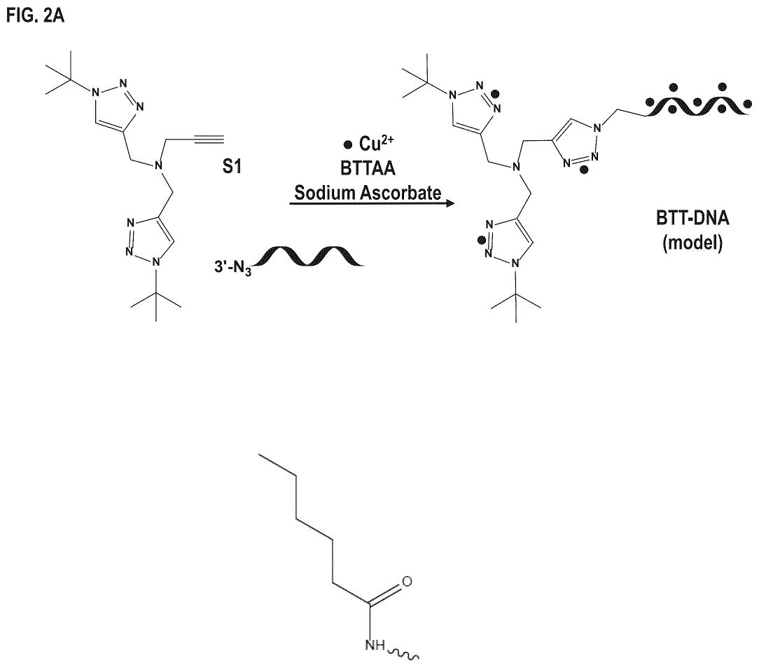

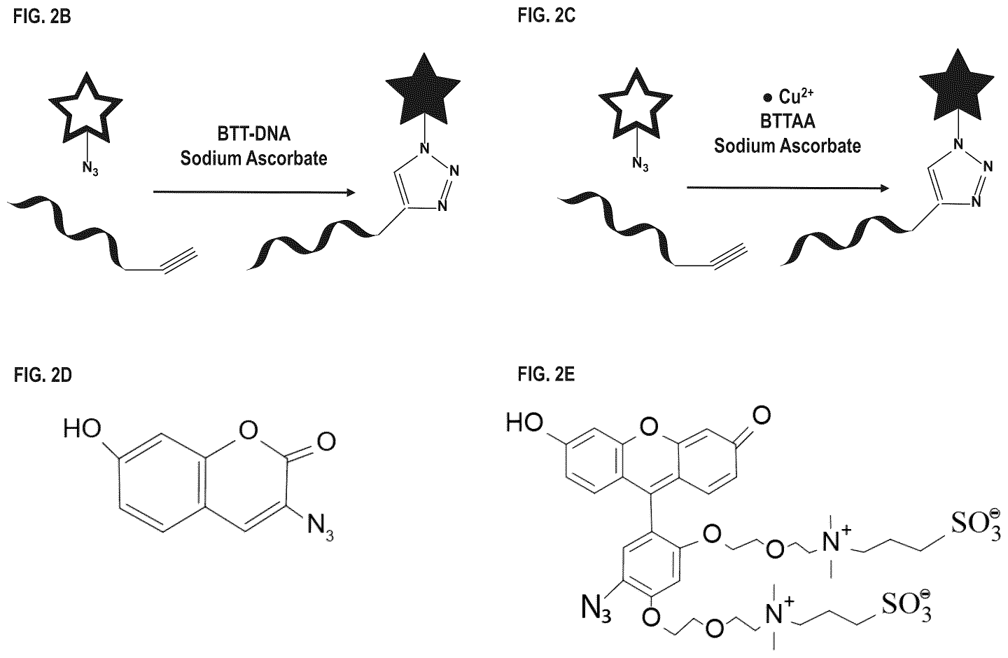

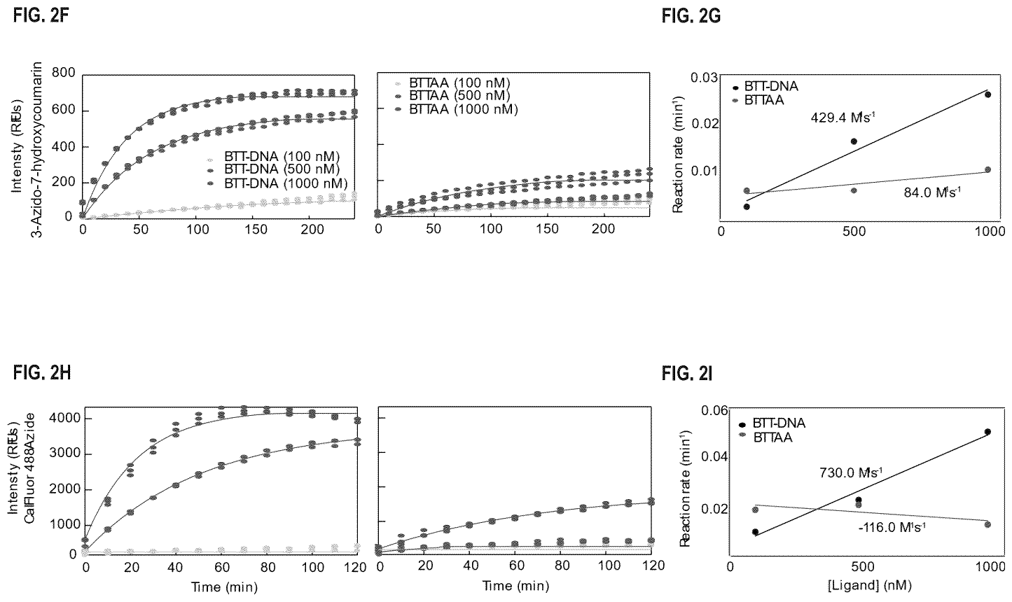

In some embodiments, a linker comprises the structure set forth in FIG. 2A. In some embodiments, BTT_{(1,2)}-DNA comprises the structure set forth in FIG. 2A. In some embodiments, BTT₍₁₎-DNA comprises the structure set forth in FIG. 2A. In some embodiments, BTT₍₂₎-DNA comprises the structure set forth in FIG. 2A. In some embodiments, BTT-DNA comprises the structure set forth in FIG. 2A. In some embodiments, BTT_{(1,2)}-DNA, BTT₍₁₎-DNA, BTT₍₂₎-DNA, BTT-DNA, or a combination thereof comprises the structure set forth in FIG. 2A. In some embodiments, a linker comprising the structure set forth in FIG. 2A is used in the synthesis of BTT_{(1,2)}-DNA. In some embodiments, a linker comprising the structure set forth in FIG. 2A is used in the synthesis of BTT₍₁₎-DNA. In some embodiments, a linker comprising the structure set forth in FIG. 2A is used in the synthesis of BTT₍₂₎-DNA. In some embodiments, a linker comprising the structure set forth in FIG. 2A is used in the synthesis of BTT-DNA. In some embodiments, a linker comprising the structure set forth in FIG. 2A is used in the synthesis of BTT_{(1,2)}-DNA, BTT₍₁₎-DNA, BTT₍₂₎-DNA, BTT-DNA, or a combination thereof.

In some embodiments, a linker is carbon-based. In some embodiments, a linker is connected to a nucleotide analog (e.g., a deoxynucleotide analog, a ribonucleotide analog, a xenonucleotide). In some embodiments, a linker comprises a nucleotide or a nucleotide analog.

In some embodiments, the CuAAC-accelerating ligand comprising a polynucleotide comprises, or further comprises, one or more copper chelators and a linker connecting the polynucleotide to each of the copper chelators.

In some embodiments, the polynucleotide of the CuAAC-accelerating ligand comprising a polynucleotide comprises DNA, RNA, XNA, a modified polynucleotide, an aptamer, or a combination thereof. In some embodiments, the polynucleotide comprises DNA. In some embodiments, the polynucleotide comprises RNA. In some embodiments, the polynucleotide comprises XNA. In some embodiments, the polynucleotide comprises a modified polynucleotide. In some embodiments, the polynucleotide comprises an aptamer.

In some embodiments, the polynucleotide comprises about 2 or more nucleotides. In some embodiments, the polynucleotide comprises about 8 or more nucleotides. In some embodiments, the polynucleotide comprises about 10 or more nucleotides. In some embodiments, the polynucleotide comprises about 15 or more nucleotides. In some embodiments, the polynucleotide comprises about 18 or more nucleotides. In some embodiments, the polynucleotide comprises about 20 or more nucleotides. In some embodiments, the polynucleotide comprises about 22 or more nucleotides. In some embodiments, the polynucleotide comprises about 30 or more nucleotides. In some embodiments, the polynucleotide comprises about 50 or more nucleotides. In some embodiments, the polynucleotide comprises about 100 or more nucleotides. In some embodiments, the polynucleotide comprises about 200 or more nucleotides. In some embodiments, the polynucleotide comprises about 2, 8, 10, 15, 18, 20, 22, 30, 50, 100, or 200 nucleotides.

In some embodiments, the polynucleotide of the CuAAC-accelerating ligand comprising a polynucleotide is connected to a linker, wherein the linker is connected to a copper chelator. In some embodiments, the polynucleotide is connected at a 3′ end to the linker. In some embodiments, the polynucleotide is connected at a 5′ end to the linker. In some embodiments, the polynucleotide is connected at an internal base to the linker. In some embodiments, the polynucleotide is connected at a 3′ end, a 5′ end, or an internal base to the linker. In some embodiments, the polynucleotide is connected at a 3′ end, a 5′ end, an internal base, or a combination thereof to more than one linker. In some embodiments, the polynucleotide is connected at a 3′ end, a 5′ end, or an internal base to a first linker. In some embodiments, the polynucleotide is connected at a 3′ end, a 5′ end, or an internal base to a second linker. In some embodiments, the polynucleotide is connected at a 3′ end, a 5′ end, or an internal base, to a first linker and a second linker.

In some embodiments, the polynucleotide of the CuAAC-accelerating ligand comprising a polynucleotide is complementary to one or more other polynucleotides. In some embodiments, the polynucleotide is complementary to a portion of one or more other polynucleotides. In some embodiments, an other polynucleotide further comprises an alkyne or an azide. In some embodiments, an other polynucleotide comprises a template (e.g., a splint, a scaffold).

In some embodiments, the polynucleotide of the CuAAC-accelerating ligand comprising a polynucleotide is conjugated (directly or indirectly) to at least one of: a nanoparticle, a small molecule (e.g., a drug), a liposome, an antibody or antigen-binding fragment thereof, a detectable molecule (e.g., that comprises a fluorophore or a fluorogenic dye), or a combination thereof. In some embodiments, the small molecule is a drug. In some embodiments, the detectable molecule is a dye, a fluorogenic dye, or a fluorophore.

In some embodiments, the polynucleotide of the CuAAC-accelerating ligand comprising a polynucleotide: comprises a single-stranded DNA, RNA, XNA, modified polynucleotide, an aptamer, or a combination thereof; is connected at a 3′ end, a 5′ end, or an internal base to a linker; binds a polypeptide; is complementary to one or more other polynucleotides; is conjugated to at least one of: a nanoparticle, a small molecule, a liposome, an antibody or antigen-binding fragment thereof, a detectable molecule, or a combination thereof; or any combination of the foregoing. In some embodiments, conjugation of a nanoparticle, a small molecule, a liposome, an antibody or antigen-binding fragment thereof, a detectable molecule, or a combination thereof to the polynucleotide is accomplished via attachment chemistry (e.g., click chemistry).

In some embodiments, the CuAAC-accelerating ligand comprising a polynucleotide: comprises one or two copper chelators; comprises one or two 2-(4-((Bis((1-(tert-butyl)-1H-1,2,3-triazol-4-yl)methyl)amino)methyl)-1H-1,2,3-triazol-1-yl) (BTT); is immobilized on the surface of a nanoparticle; further comprises a nanoparticle, a small molecule, a liposome, an antibody or antigen-binding fragment thereof, a detectable molecule, a chemical functional group, or a combination thereof; chelates about 7 to 10 copper atoms; or any combination of the foregoing. In some embodiments, conjugation of a nanoparticle, a small molecule, a liposome, an antibody or antigen-binding fragment thereof, a detectable molecule, or a combination thereof to the ligand is accomplished via attachment chemistry (e.g., click chemistry).

In some embodiments, the CuAAC-accelerating ligand comprising a polynucleotide comprises one or two copper chelators. In some embodiments, the CuAAC-accelerating ligand comprising a polynucleotide comprises one copper chelator. In some embodiments, the CuAAC-accelerating ligand comprising a polynucleotide comprises two copper chelators.

In some embodiments, the CuAAC-accelerating ligand comprising a polynucleotide comprises one or two 2-(4-((Bis((1-(tert-butyl)-1H-1,2,3-triazol-4-yl)methyl)amino)methyl)-1H-1,2,3-triazol-1-yl) (BTT). In some embodiments, the CuAAC-accelerating ligand comprising a polynucleotide comprises one 2-(4-((Bis((1-(tert-butyl)-1H-1,2,3-triazol-4-yl)methyl)amino)methyl)-1H-1,2,3-triazol-1-yl) (BTT). In some embodiments, the CuAAC-accelerating ligand comprising a polynucleotide comprises two 2-(4-((Bis((1-(tert-butyl)-1H-1,2,3-triazol-4-yl)methyl)amino)methyl)-1H-1,2,3-triazol-1-yl) (BTT).

In some embodiments, the CuAAC-accelerating ligand comprising a polynucleotide is immobilized on the surface of a nanoparticle. In some embodiments, the CuAAC-accelerating ligand comprising a polynucleotide further comprises a nanoparticle, a small molecule, a liposome, an antibody or antigen-binding fragment thereof, a detectable molecule, a chemical functional group, or a combination thereof. In some embodiments, the small molecule is a drug. In some embodiments, the detectable molecule is a dye, a fluorogenic dye, or a fluorophore.

In some embodiments, the CuAAC-accelerating ligand comprising a polynucleotide chelates about 7 to 10 copper atoms. In some embodiments, the CuAAC-accelerating ligand comprising a polynucleotide chelates about 10 or more copper atoms. In some embodiments, the CuAAC-accelerating ligand comprising a polynucleotide chelates about 7, 8, 9, 10, 7-8, 8-9, or 9-10 copper atoms. In some embodiments, the CuAAC-accelerating ligand comprising a polynucleotide chelates an average of 7.5 copper atoms. In some embodiments, the CuAAC-accelerating ligand comprising a polynucleotide chelates about 7.5 copper atoms. In some embodiments, the CuAAC-accelerating ligand comprising a polynucleotide chelates an average of 10 copper atoms. In some embodiments, the CuAAC-accelerating ligand comprising a polynucleotide chelates about 10 copper atoms.

In some embodiments, the CuAAC-accelerating ligand comprising a polynucleotide further comprises a nanoparticle, a small molecule (e.g., a drug), a liposome, an antibody or antigen-binding fragment thereof, a detectable molecule (e.g., that comprises a fluorophore or a fluorogenic dye), a chemical functional group (e.g., an alkyne), or a combination thereof.

### Methods of Synthesis

In another aspect, the disclosure herein provides a method of synthesizing a Cu(I)-catalyzed azide-alkyne cycloaddition (CuAAC)-accelerating ligand comprising a polynucleotide (i.e., a CuAAC-accelerating ligand, wherein the ligand comprises a polynucleotide). In some embodiments, the method comprises the steps of:
a) preparing a CuAAC reaction mixture;
b) adding sodium ascorbate to the CuAAC reaction mixture; and
c) shaking the CuAAC reaction mixture,
   thereby driving the Cu(I)-catalyzed cycloaddition of the azide-labeled polynucleotide to the copper chelator to produce the CuAAC-accelerating ligand comprising a polynucleotide.

In some embodiments, a method of synthesizing a (CuAAC)-accelerating ligand comprising a polynucleotide comprises the steps of:
a) preparing a CuAAC reaction mixture by combining:
   i) an azide-labeled polynucleotide;
   ii) a copper chelator, wherein the copper chelator comprises an alkyne; and
   iii) a copper source;
a) adding sodium ascorbate to the CuAAC reaction mixture; and
b) shaking the CuAAC reaction mixture, thereby driving the Cu(I)-catalyzed cycloaddition of the azide-labeled polynucleotide to the copper chelator to produce the CuAAC-accelerating ligand comprising a polynucleotide.

In some embodiments, a CuAAC reaction mixture comprises an azide-labeled polynucleotide; a copper chelator, wherein the copper chelator comprises an alkyne; and a copper source. In some embodiments, sodium ascorbate is added to the CuAAC reaction mixture, thereby producing a sodium ascorbate-CuAAC reaction mixture. In some embodiments, the sodium ascorbate-CuAAC reaction mixture comprises an azide-labeled polynucleotide; a copper chelator, wherein the copper chelator comprises an alkyne; a copper source; and sodium ascorbate. In some embodiments, the mixture (i.e., the CuAAC reaction mixture or the sodium ascorbate-CuAAC reaction mixture) further comprises spermine. In some embodiments, the mixture (i.e., the CuAAC reaction mixture or the sodium ascorbate-CuAAC reaction mixture) further comprises sodium chloride (NaCl). In some embodiments, the mixture (i.e., the CuAAC reaction mixture or the sodium ascorbate-CuAAC reaction mixture) further comprises spermine and NaCl.

In some embodiments, the azide-labeled polynucleotide comprises one or two azide groups. In some embodiments, the azide-labeled polynucleotide comprises one azide group. In some embodiments, the azide-labeled polynucleotide comprises two azide groups. In some embodiments, the one or two azide groups are at the 5′ end of the azide-labeled polynucleotide. In some embodiments, the one or two azide groups are at the 3′ end of the azide-labeled polynucleotide. In some embodiments, the one or two azide groups are connected to one or two internal bases of the azide-labeled polynucleotide. In some embodiments, one azide group is connected to one internal base of the azide-labeled polynucleotide. In some embodiments, a first azide group is connected to a first internal base of the azide-labeled polynucleotide. In some embodiments, a second azide group is connected to a second internal base of the azide-labeled polynucleotide. In some embodiments, a first azide group is connected to a first internal base of the azide-labeled polynucleotide and a second azide group is connected to a second internal base of the azide-labeled polynucleotide. In some embodiments, the azide-labeled polynucleotide is connected to more than two azide groups. In some embodiments, the azide-labeled polynucleotide is connected to the more than two azide groups at the 5′ end of the azide-labeled polynucleotide, at the 3′ end of the azide-labeled polynucleotide, at one or more internal bases of the azide-labeled polynucleotide, or a combination of the foregoing.

In some embodiments, the azide-labeled polynucleotide is synthesized. In some embodiments, the azide-labeled polynucleotide is synthesized by using a transferase enzyme to add an azido-nucleotide to an oligomer (e.g., a 15-mer). In some embodiments, the transferase enzyme is a terminal deoxynucleotidyl transferase (TdT). In some embodiments, the azido-nucleotide is an azido-deoxyuridine treiphosphate (azido dUTP). In some embodiments, one azido-nucleotide is added to an oligomer to generate the azide-labeled polynucleotide. In some embodiments, two azido-nucleotides are added to an oligomer to generate the azide-labeled polynucleotide. In some embodiments, an azido-nucleotide comprises a linker. In some embodiments, an azido-nucleotide comprises azide-PEG4-aminoallyl-dUTP.

In some embodiments, the CuAAC reaction mixture for synthesizing a CuAAC-accelerating ligand comprising a polynucleotide comprises a copper chelator. In some embodiments, the copper chelator is biocompatible. In some embodiments, the copper chelator comprises an alkyne. In some embodiments, the copper chelator comprises one or more tert-butyl groups. In some embodiments, the copper chelator is biocompatible, comprises an alkyne, comprises one or more tert-butyl groups, or a combination thereof. In some embodiments, a copper chelator is modified to be biocompatible, to comprise an alkyne, to comprise one or more tert-butyl groups, or a combination thereof. Non-limiting examples of copper chelators include S1 alkyne (S1; (N,N-bis((1-tert-butyl-1H-1,2,3-triazol-4-yl)methyl) prop-2-yn-1-amine; CAS No. 1257633-68-3), BTTAA (2-(4-((Bis((1-(tert-butyl)-1H-1,2,3-triazol-4-yl)methyl)amino)methyl)-1H-1,2,3-triazol-1-yl) acetic acid; CAS No: 1334179-85-9), and BTTES-Cu(I) (see, for example, Soriano del Amo et al. (2010) and Wu et al. (2012), the entire contents of which are incorporated herein by reference). In some embodiments, the copper chelator is S1.

In some embodiments, the CuAAC reaction mixture for synthesizing a CuAAC-accelerating ligand comprising a polynucleotide comprises a copper source. In some embodiments, the copper source is copper sulfate (CuSO₄).

In some embodiments, the CuAAC reaction mixture for synthesizing a CuAAC-accelerating ligand comprising a polynucleotide further comprises a CuAAC-accelerating ligand. In some embodiments, the CuAAC-accelerating ligand comprises BTTAA. In some embodiments, the CuAAC-accelerating ligand comprises the CuAAC-accelerating ligand comprising a polynucleotide.

In some embodiments, the CuAAC reaction mixture for synthesizing a CuAAC-accelerating ligand comprising a polynucleotide comprises sodium ascorbate at a molar concentration about 417-fold greater than the molar concentration of the azide-labeled polynucleotide.

In some embodiments, the method of synthesizing a CuAAC-accelerating ligand comprising a polynucleotide comprises shaking. In some embodiments, the shaking is performed by an orbital shaker. In some embodiments, the method comprises shaking at about 600 rpm. In some embodiments, the shaking is performed by an orbital shaker at about 600 rpm. In some embodiments, the method comprises gentle shaking. In some embodiments, the method comprises vigorous shaking. In some embodiments, the method comprises shaking at more than 600 rpm. In some embodiments, the method comprises shaking at less than 600 rpm. In some embodiments, the method comprises shaking at about 0° C.-37° C. In some embodiments, the method comprises shaking at about 0° C. In some embodiments, the method comprises shaking at about 37° C. In some embodiments, the method comprises shaking at 0° C. In some embodiments, the method comprises shaking at 37° C. In some embodiments, the method comprises shaking at a temperature of at least 37° C.

In some embodiments, the method of synthesizing a CuAAC-accelerating ligand comprising a polynucleotide comprises, or further comprises, purifying the CuAAC-accelerating ligand comprising a polynucleotide from a remainder of the CuAAC reaction mixture. In some embodiments, the purifying is performed: with 3.5 kD molecular weight cut-off, or molecular weight threshold, dialysis tubing; for about 24 hours; in about 3.5 L of water; at room temperature; or a combination thereof. In some embodiments, the purifying is performed with 3.5 kD molecular weight cut-off, or molecular weight threshold, dialysis tubing for about 24 hours in about 3.5 L of water at room temperature.

In some embodiments, the method of synthesizing a CuAAC-accelerating ligand comprising a polynucleotide comprises:
a) preparing a CuAAC reaction mixture, wherein the reaction mixture comprises, or further comprises:
   i. an azide-labeled polynucleotide comprises one or two azide groups;
   ii. a biocompatible copper chelator;
   iii. an S1 alkyne;
   iv. copper sulfate;
   v. a CuAAC-accelerating ligand;
   vi. sodium ascorbate present at a molar concentration about 417-fold greater than the molar concentration of the azide-labeled polynucleotide; or
   vii. a combination thereof;
b) shaking the CuAAC reaction mixture, wherein the shaking:
   i. is performed by an orbital shaker;
   ii. is performed at about 600 rpm;
   iii. is performed at about 0° C. to 37° C.; or
   iv. a combination of the foregoing;
c) purifying the CuAAC-accelerating ligand comprising a polynucleotide from a remainder of the CuAAC reaction mixture; or
d) any combination of the foregoing.

In some embodiments, the method of synthesizing a CuAAC-accelerating ligand comprising a polynucleotide comprises:
a) preparing a CuAAC reaction mixture comprising:
   i. a copper source, wherein the copper source is copper sulfate, wherein the copper sulfate is present at a molar concentration about 13-fold greater than the molar concentration of the azide-labeled polynucleotide;
   ii. a CuAAC-accelerating ligand, wherein:
      1) the CuAAC-accelerating ligand is present at a molar concentration about 25-fold greater than the molar concentration of the azide-labeled polynucleotide;
      2) the CuAAC-accelerating ligand comprises BTTAA; or
      3) both of the foregoing;
   b) purifying the CuAAC-accelerating ligand comprising a polynucleotide from a remainder of the CuAAC reaction mixture, wherein the purifying is performed with 3.5 kD molecular weight cut-off dialysis tubing for about 24 hours in about 3.5 L of water at room temperature; or
   c) any combination of the foregoing.

In some embodiments, a method of synthesizing a CuAAC-accelerating ligand comprising a polynucleotide comprises preparing a CuAAC reaction mixture comprising:
a) an azide-labeled polynucleotide;
b) copper sulfate present at a molar concentration about 13-fold greater than the molar concentration of the azide-labeled polynucleotide;
c) sodium ascorbate present at a molar concentration about 417-fold greater than the molar concentration of the azide-labeled polynucleotide;
d) a CuAAC-accelerating ligand present at a molar concentration about 25-fold greater than the molar concentration of the azide-labeled polynucleotide;
e) copper chelator present at a molar concentration about 100-fold to 200-fold greater than the molar concentration of the azide-labeled polynucleotide; or
f) a combination of the foregoing.

In some embodiments, a method of synthesizing a CuAAC-accelerating ligand comprising a polynucleotide comprises preparing a CuAAC reaction mixture comprising at least one azide-labeled polynucleotide that comprises one azide group; and at least one azide-labeled polynucleotide that comprises two azide groups.

In some embodiments, a method of synthesizing a CuAAC-accelerating ligand comprising a polynucleotide comprises preparing a CuAAC reaction mixture that comprises copper chelator present at a molar concentration greater than, e.g., about 100-fold greater than, the molar concentration of the azide-labeled polynucleotide. In some embodiments, a method of synthesizing a CuAAC-accelerating ligand comprising a polynucleotide comprises shaking the CuAAC reaction mixture, e.g., wherein the shaking is performed for about 60 minutes. In some embodiments, a method of synthesizing a CuAAC-accelerating ligand comprising a polynucleotide comprises: preparing a CuAAC reaction mixture comprises copper chelator present at a molar concentration about 100-fold greater than the molar concentration of the azide-labeled polynucleotide; and shaking the CuAAC reaction mixture, wherein the shaking is performed for about 60 minutes.

In some embodiments, a method of synthesizing a CuAAC-accelerating ligand comprising a polynucleotide comprises preparing a CuAAC reaction mixture that comprises: azide-labeled polynucleotide, S1 alkyne, copper sulfate, BTTAA, sodium ascorbate, or any combination thereof. In some embodiments, a method of synthesizing a CuAAC-accelerating ligand comprising a polynucleotide comprises preparing a CuAAC reaction mixture that comprises: about 6 μM azide-labeled polynucleotide, about 600 μM S1 alkyne, about 75 μM copper sulfate, about 150 μM BTTAA, about 2.5 mM sodium ascorbate, or any combination thereof. In some embodiments, the CuAAC reaction mixture comprises about 6 μM azide-labeled polynucleotide. In some embodiments, the CuAAC reaction mixture comprises about 600 μM S1 alkyne. In some embodiments, the CuAAC reaction mixture comprises about 75 μM copper sulfate. In some embodiments, the CuAAC reaction mixture comprises about 150 μM BTTAA. In some embodiments, the CuAAC reaction mixture comprises about 2.5 mM sodium ascorbate. In some embodiments, the CuAAC reaction mixture comprises about 6 μM azide-labeled polynucleotide, about 600 μM S1 alkyne, about 75 μM copper sulfate, about 150 μM BTTAA, and about 2.5 mM sodium ascorbate.

In some embodiments, a method of synthesizing a CuAAC-accelerating ligand comprising a polynucleotide comprises preparing a CuAAC reaction mixture that comprises: azide-labeled polynucleotide, S1 alkyne, copper sulfate, BTTAA, sodium ascorbate, or any combination thereof. In some embodiments, a method of synthesizing a CuAAC-accelerating ligand comprising a polynucleotide comprises preparing a CuAAC reaction mixture that comprises: 6 μM azide-labeled polynucleotide, 600 μM S1 alkyne, 75 μM copper sulfate, 150 μM BTTAA, 2.5 mM sodium ascorbate, or any combination thereof. In some embodiments, the CuAAC reaction mixture comprises 6 μM azide-labeled polynucleotide. In some embodiments, the CuAAC reaction mixture comprises 600 μM S1 alkyne. In some embodiments, the CuAAC reaction mixture comprises 75 μM copper sulfate. In some embodiments, the CuAAC reaction mixture comprises 150 μM BTTAA. In some embodiments, the CuAAC reaction mixture comprises 2.5 mM sodium ascorbate. In some embodiments, the CuAAC reaction mixture comprises 6 μM azide-labeled polynucleotide, 600 μM S1 alkyne, 75 μM copper sulfate, 150 μM BTTAA, and 2.5 mM sodium ascorbate.

In some embodiments, a method of synthesizing a CuAAC-accelerating ligand comprising a polynucleotide comprises preparing a CuAAC reaction mixture that comprises copper chelator present at a molar concentration about 200-fold greater than the molar concentration of the azide-labeled polynucleotide. In some embodiments, a method of synthesizing a CuAAC-accelerating ligand comprising a polynucleotide comprises shaking the CuAAC reaction mixture, wherein the shaking is performed for about 30 minutes. In some embodiments, a method of synthesizing a CuAAC-accelerating ligand comprising a polynucleotide comprises: preparing a CuAAC reaction mixture comprises copper chelator present at a molar concentration about 200-fold greater than the molar concentration of the azide-labeled polynucleotide; and shaking the CuAAC reaction mixture, wherein the shaking is performed for about 30 minutes.

In some embodiments, a method of synthesizing a CuAAC-accelerating ligand comprising a polynucleotide comprises preparing a CuAAC reaction mixture that comprises: about 6 μM azide-labeled polynucleotide, about 1200 μM S1 alkyne, about 75 M copper sulfate, about 150 μM BTTAA, about 2.5 mM sodium ascorbate, or any combination thereof. In some embodiments, the CuAAC reaction mixture comprises about 6 μM azide-labeled polynucleotide. In some embodiments, the CuAAC reaction mixture comprises about 1200 μM S1 alkyne. In some embodiments, the CuAAC reaction mixture comprises about 75 μM copper sulfate. In some embodiments, the CuAAC reaction mixture comprises about 150 μM BTTAA. In some embodiments, the CuAAC reaction mixture comprises about 2.5 mM sodium ascorbate. In some embodiments, the CuAAC reaction mixture comprises about 6 μM azide-labeled polynucleotide, about 1200 μM S1 alkyne, about 75 μM copper sulfate, about 150 μM BTTAA, and about 2.5 mM sodium ascorbate.

In some embodiments, a method of synthesizing a CuAAC-accelerating ligand comprising a polynucleotide comprises preparing a CuAAC reaction mixture that comprises: 6 μM azide-labeled polynucleotide, 1200 μM S1 alkyne, 75 μM copper sulfate, 150 μM BTTAA, 2.5 mM sodium ascorbate, or any combination thereof. In some embodiments, the CuAAC reaction mixture comprises 6 μM azide-labeled polynucleotide. In some embodiments, the CuAAC reaction mixture comprises 1200 μM S1 alkyne. In some embodiments, the CuAAC reaction mixture comprises 75 μM copper sulfate. In some embodiments, the CuAAC reaction mixture comprises 150 μM BTTAA. In some embodiments, the CuAAC reaction mixture comprises 2.5 mM sodium ascorbate. In some embodiments, the CuAAC reaction mixture comprises 6 μM azide-labeled polynucleotide, 1200 μM S1 alkyne, 75 μM copper sulfate, 150 UM BTTAA, and 2.5 mM sodium ascorbate.

### Methods of Use

In another aspect, the disclosure provides a method of ligating a payload to a target, the method comprising performing a Cu(I)-catalyzed azide-alkyne cycloaddition (CuAAC) of the payload to the target in the presence of a CuAAC-accelerating ligand comprising a polynucleotide.

In some embodiments, the payload comprises an azide and the target comprises an alkyne. In some embodiments, the payload comprises and alkyne and the target comprises an azide. In some embodiments, the target comprises a nanoparticle, a small molecule, a liposome, an antibody or antigen-binding fragment thereof, a detectable molecule, a chemical functional group, or a combination thereof. In some embodiments, the payload comprises a nanoparticle, a small molecule, a liposome, an antibody or antigen-binding fragment thereof, a detectable molecule, a chemical functional group, or a combination thereof.

In some embodiments, the CuAAC occurs at (i.e., use of the CuAAC-accelerating ligand leads to) a rate of at least 1×10⁻⁵ min⁻¹ nM⁻¹. In some embodiments, use of the CuAAC-accelerating ligand comprising a polynucleotide leads to a reaction rate of at least 1×10⁻⁵ min⁻¹ nM⁻¹. In some embodiments, use of the CuAAC-accelerating ligand comprising a polynucleotide leads to a reaction rate of about 1×10⁻⁵ min⁻¹ nM⁻¹. In some embodiments, use of the CuAAC-accelerating ligand comprising a polynucleotide leads to a reaction rate of at least about 1×10⁻⁵ min⁻¹ nM⁻¹.

In some embodiments, a reaction rate (e.g., of a CuAAC) is about 2.58×10⁻⁵ min⁻¹ nM⁻¹. In some embodiments, a reaction rate is at least 2.58×10⁻⁵ min⁻¹ nM⁻¹. In some embodiments, a reaction rate is at least about 2.58×10⁻⁵ min⁻¹ nM⁻¹. In some embodiments, a reaction rate is about 2.58×10⁻⁵ min⁻¹ nM⁻¹ when the azide component of the CuAAC (i.e., the payload) comprises a coumarin azide dye. In some embodiments, a reaction rate is at least 2.58×10⁻⁵ min⁻¹ nM⁻¹ when the azide component of the CuAAC (i.e., the payload) comprises a coumarin azide dye. In some embodiments, a reaction rate is at least about 2.58×10⁻⁵ min⁻¹ nM⁻¹ when the azide component of the CuAAC (i.e., the payload) comprises a coumarin azide dye.

In some embodiments, a reaction rate (e.g., of a CuAAC) is about 4.38×10⁻⁵ min⁻¹ nM⁻¹. In some embodiments, a reaction rate is at least 4.38×10⁻⁵ min⁻¹ nM⁻¹. In some embodiments, a reaction rate is at least about 4.38×10⁻⁵ min⁻¹ nM⁻¹. In some embodiments, a reaction rate is about 4.38×10⁻⁵ min⁻¹ nM⁻¹ when the azide component of the CuAAC (i.e., the payload) comprises a CalFluor azide dye. In some embodiments, a reaction rate is at least 4.38×10⁻⁵ min⁻¹ nM⁻¹ when the azide component of the CuAAC (i.e., the payload) comprises a CalFluor azide dye. In some embodiments, a reaction rate is at least about 4.38×10⁻⁵ min⁻¹ nM⁻¹ when the azide component of the CuAAC (i.e., the payload) comprises a CalFluor azide dye.

In some embodiments, the CuAAC-accelerating ligand comprising a polynucleotide accelerates a reaction rate when used at nanomolar concentrations (i.e., concentrations in the nanomolar range).

In some embodiments, the CuAAC occurs: inside or on the surface of a live cell, a fixed cell, a dead cell, or a combination thereof; inside or on the surface of a population, an organoid, or a tissue comprising a live cell, a fixed cell, a dead cell, or a combination; and/or in a living subject. In some embodiments, the CuAAC occurs inside or on the surface of a live cell. In some embodiments, the CuAAC occurs inside or on the surface of a dead cell. In some embodiments, the CuAAC occurs inside or on the surface of a fixed cell. In some embodiments, the CuAAC occurs inside or on the surface of a live cell, a dead cell, a fixed cell, or a combination thereof. In some embodiments, the CuAAC occurs in a living subject.

In some embodiments, the CuAAC occurs inside a live cell.

In some embodiments, the CuAAC reagents (i.e., the ligand, the target, the payload) are delivered into a cell. In some embodiments, the CuAAC reagents further comprise a template, spermine, salt, sodium ascorbate, or a combination thereof. In some embodiments, delivery of CuAAC reagents into a cell is accomplished by passive means (e.g., passive uptake). In some embodiments, delivery of CuAAC reagents into a cell is accomplished by electroporation. In some embodiments, delivery of CuAAC reagents into a cell is accomplished by lipofection.

In some embodiments, the target comprises:
a) an alkyne-derivatized thymidine analog, thereby enabling detection of nascent DNA in cells;
b) an alkyne-derivatized uridine analog, thereby enabling detection of nascent RNA in cells;
c) an alkyne-derivatized methionine analog, thereby enabling detection of nascent protein on or in cells;
d) an alkyne-derivatized monosaccharide, thereby enabling detection of sialylated glycans (e.g., glycans that comprise sialic acid) on or in cells;
e) an alkyne-derivatized choline, thereby enabling detection of choline-containing phospholipids on or in cells;
f) a library of alkyne-containing compounds; or
g) a combination of the foregoing.

In some embodiments, the target comprises:
a) 5-ethynyl-2′-deoxyuridine (EdU);
b) 5-ethynyl uridine (EU);
c) L-homopropargyl (L-HPG);
d) N-(4-pentynoyl) mannosamine (Ac₄MaNAl; CAS No. 935658-93-8);
e) propargyl choline; or
f) a combination of the foregoing.

In some embodiments, the target comprises:
a) an alkyne-derivatized thymidine analog, such as 5-ethynyl-2′-deoxyuridine (EdU), thereby enabling detection of nascent DNA in cells;
b) an alkyne-derivatized uridine analog, such as 5-ethynyl uridine (EU), thereby enabling detection of nascent RNA in cells;
c) an alkyne-derivatized methionine analog, such as L-homopropargyl (L-HPG), thereby enabling detection of nascent protein on or in cells;
d) an alkyne-derivatized monosaccharide, such as N-(4-pentynoyl) mannosamine (Ac₄MaNAl), thereby enabling detection of sialylated glycans on or in cells;
e) an alkyne-derivatized choline, such as propargyl choline, thereby enabling detection of choline-containing phospholipids on or in cells;
f) a library of alkyne-containing compounds; or
g) a combination of the foregoing.

In some embodiments, performing the CuAAC of the payload to the target in the presence of a CuAAC-accelerating ligand comprises the steps of: contacting a cell, a population of cells, an organoid, or a tissue with the target, wherein the cell, the population of cells, the organoid, or the tissue comprises a live cell, a fixed cell, a dead cell, or a combination thereof, and wherein the target comprises an alkyne-containing compound or drug; further contacting the cell, the population of cells, the organoid, or the tissue with the payload and the CuAAC-accelerating ligand comprising a nucleotide, wherein the payload comprises a detectable molecule comprising an azide, thereby producing a compound or drug ligated to a detectable molecule; and detecting the detectable molecule, its location, or both the detectable molecule and its location. In some embodiments, the detectable is a fluorogenic azide. In some embodiments, the target further comprises a library of alkyne-containing compounds or drugs.

In some embodiments, the method further comprises performing the CuAAC in the presence of a template, wherein the template or a portion thereof is complementary to: the polynucleotide of the ligand or a portion thereof; and the molecule or payload or a portion thereof, and/or the target or a portion thereof. In some embodiments, the method further comprises performing the CuAAC in the presence of spermine.

In yet another aspect, the disclosure provides a method of labeling a live cell, the method comprising performing a CuAAC of a label to a target in the presence of a CuAAC-accelerating ligand comprising a polynucleotide (i.e., a CuAAC-accelerating ligand, wherein the ligand comprises a polynucleotide) in a live cell. In some embodiments, the target is an RNA comprising an alkyne-derivatized uridine analog.

In yet another aspect, the disclosure provides a method of intracellular labeling, the method comprising performing a CuAAC of a label to a target in the presence of a CuAAC-accelerating ligand comprising a polynucleotide (i.e., a CuAAC-accelerating ligand, wherein the ligand comprises a polynucleotide) in a live cell. In some embodiments, the target is an RNA comprising an alkyne-derivatized uridine analog. In some embodiments, the target is DNA comprising an alkyne-derivatized thymidine analog.

In yet another aspect, the disclosure herein provides a kit comprising a CuAAC-accelerating ligand comprising a polynucleotide, a target, and a payload. In some embodiments, the kit further comprises a template. In some embodiments, the kit further comprises spermine. In some embodiments, the kit further comprises buffers.

In some embodiments, the kit is useful for detection of biomolecules (e.g., DNA, RNA, protein, cell surface glycans, phospholipids). In some embodiments, the target and/or the payload comprises a detectable molecule (i.e., a tag or a label, e.g., a fluorophore or a fluorogenic azide).

In some embodiments, the kit is useful for delivery of a therapy or therapeutic agent (e.g., a drug, a pharmaceutical composition). In some embodiments, the ligand, the target, and/or the payload comprises a therapy or a therapeutic agent. In some embodiments, a therapy or a therapeutic agent comprises the ligand, the target, and/or the payload. In some embodiments, the ligand, the target, and/or the payload are pharmaceutically acceptable for use as, comprising, or in conjunction with, a therapeutic agent. In some embodiments, a therapeutically effective amount of a therapeutic agent is delivered. In some embodiments, the ligand, the target, the payload, and/or the template aids in delivering the therapeutic agent to an intended location (e.g., the location of a particular protein) in a subject.

### General Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., ±10%, ±5% or ±1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours. As used herein, the symbol “+” denotes a range, that is, where a given value is “N±X” a range from N−X to N±X, wherein the range includes the values of N−X and N+X.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

When introducing elements disclosed herein, the articles “a,” “an,” “the,” and “said” are intended to mean that there are one or more of the elements. Further, the one or more elements may be the same or different. Thus, for example, unless the context clearly indicates otherwise, “an agent” includes a single agent, and two or more agents. Further the two or more agents can be the same or different as, for example, in embodiments wherein a first agent comprises a polynucleotide of a first sequence and a second agent comprises a polynucleotide of a second sequence.

As used herein, “increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

As used herein, “increases” also means increases by at least 1-fold, for example, 1-, 2-, 3—, 4-, 5-, 6-, 7-, 8-, 9-, 10-, 15-, 20-, 30-, 40-, 50-, 60-, 70-, 80-, 90-, 100-, 200-, 500-, or 1000-fold or more, for example, as compared to the level of a as compared to the level of a reference standard.

As used herein, “decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99, or 100%, for example, as compared to the level of reference.

As used herein, “decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

As used herein, the term “reference” means a standard or control condition (e.g., untreated with a test agent or combination of test agents). Alternatively, “reference” may refer to a resource, such as an annotated genome, transcriptome, or the like, that is used to assemble, analyze, and/or interpret data.

As used herein, the term “eliminate” means to decrease to a level that is undetectable.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “isolated” refers to a material that is free to varying degrees from components which normally accompany it as found in its native state. “Isolate” denotes a degree of separation from original source or surroundings.

### Click Chemistry

As used herein, “click chemistry” or “click reaction” refers to a Cu(I)-catalyzed azide-alkyne cycloaddition (CuAAC). CuAAC provides a method of covalently binding a first and a second molecule together, wherein the first molecule comprises an azide and the second molecule comprises an alkyne. A CuAAC may be interchangeably referred to as a ligation, ligating, binding, a conjugation, conjugating, a connection, connecting, an addition, adding, clicking, snapping, a combination, combining, building, joining, merging, piecing together, or other similar terms.

Copper in the oxidation state of Cu(I) is the active oxidation state that is used to catalyze CuAAC. As used herein, the terms “copper” and “Cu(I)” may be used interchangeably; the terms “Cu(I)” and “Cu¹⁺” may be used interchangeably; and the terms “Cu(II)” and “Cu²⁺” may be used interchangeably. A copper source in a CuAAC is represented herein as “Cu¹⁺” or “Cu²⁺.” As used herein, a “copper source” refers to a source (e.g., a reagent, e.g., copper sulfate) that provides copper in the active oxidation state that is used to catalyze CuAAC, or in an oxidation state that can be reduced to the active oxidation state that is used to catalyze CuAAC (e.g., a copper source may provide Cu²⁺ (Cu(II)), which is then reduced to Cu¹⁺ (Cu(I))). In some embodiments, a reductant (e.g., sodium ascorbate) is required to enable copper from a copper source to catalyze CuAAC.

CuAAC-accelerating ligands, also referred to as CuAAC ligands, are used in the art to accelerate the rate of a CuAAC and stabilize the Cu(I) oxidation state in aqueous solution. The stabilization of the Cu(I) oxidation state occurs via chelation of copper by the CuAAC ligands. Chelation is a non-covalent coordination between atoms that can donate electron density (such as, but not limited to, nitrogen, oxygen, sulfur, and phosphorus) to a metal (e.g., Cu(I)). The stabilization of the Cu(I) oxidation state occurs by a CuAAC ligands can accelerate the rate of a CuAAC reaction (see, for example, Hein and Fokin (2010)). Known CuAAC-accelerating ligands are referred to herein as “commercially available.” An example of a commercially available CuAAC-accelerating ligand is BTTAA (2-(4-((Bis((1-(tert-butyl)-1H-1,2,3-triazol-4-yl)methyl)amino)methyl)-1H-1,2,3-triazol-1-yl) acetic acid; CAS No: 1334179-85-9). Additional non-limiting examples of CuAAC-accelerating ligands include TBTA (tris(benzyltriazolyl)methyl amine; CAS No. 510758-28-8), BTTES (3-(4-((Bis((1-(tert-butyl)-1H-1,2,3-triazol-4-yl)methyl)amino)methyl)-1H-1,2,3-triazol-1-yl) propane-1-sulfonic acid; CAS No. 2101505-88-6), and THPTA (Tris((1-hydroxy-propyl-1H-1,2,3-triazol-4-yl)methyl)amine; CAS No. 760952-88-3).

Embodiments of the instant invention disclosed herein comprise CuAAC-accelerating ligands, including BTT₍₁₎-DNA, BTT₍₂₎-DNA, BTT_{(1,2)}-DNA (a mixture of BTT₍₁₎-DNA and BTT₍₂₎-DNA), and BTT-DNA. The terms “DNA oligomer-conjugated CuAAC ligand,” “DNA-enhanced CuAAC ligand,” “new ligand,” “CuAAC-accelerating ligand, wherein the ligand comprises a polynucleotide,” “CuAAC-accelerating ligand comprising a polynucleotide,” and other similar terms are used interchangeably to refer to embodiments of the instant invention disclosed herein.

In some embodiments, a CuAAC-accelerating ligand comprising a polynucleotide, a target, and/or a payload comprise a tag, a label, and/or a dye. In some embodiments, a tag comprises a dye. In some embodiments, a dye comprises a tag. In some embodiments, a tag does not comprise a dye. As used herein, the terms “dye,” “tag,” and “label” may be used interchangeably.

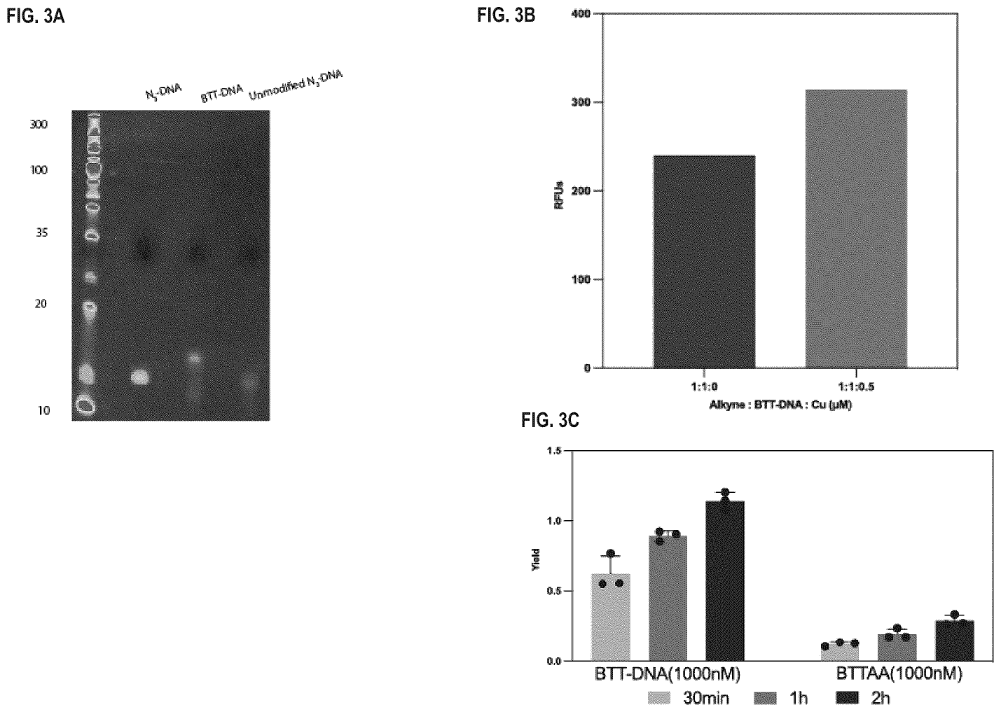

In some embodiments, a dye comprises a fluorogenic dye. In some embodiments, a dye comprises a fluorogenic azide. As used herein, the terms “fluorogenic dye” and “fluorogenic azide” are used interchangeably. Non-limiting examples of fluorogenic azide dyes include CalFluor 488 azide (CAS No. 1798305-98-2; for structure, see FIG. 2E) and 3-Azido-7-hydroxycoumarin (CAS No. 817638-68-9; for structure, see FIG. 2D). Additional examples of fluorogenic dyes include CalFluor 647 azide (CAS No. 1798306-01-0), CalFluor 555 azide (CAS No. 1798305-99-3), and CalFluor 580 azide (CAS No. 1798306-00-9).

### Nucleic Acids

As disclosed herein, deoxyribonucleic acid (DNA), ribonucleic acid (RNA), and xeno nucleic acid (XNA) may comprise natural, synthetic, or modified nucleotides, or a combination thereof. For further description of modified nucleotides (e.g., modified DNA, modified DNA bases), see, for example, Bilyard, Becker, and Balasubramanian (2020). For further description of XNA, see, for example, Duffy, Arangundy, and Holliger (2020).

Embodiments of a CuAAC-accelerating ligand comprising a polynucleotide are disclosed herein. As disclosed herein, the polynucleotide is interchangeably referred to as a polynucleotide attachment, oligomer, oligomer attachment, DNA attachment, DNA oligomer, DNA scaffold, and similar terms. In some embodiments, the oligomer attachment comprises DNA, RNA, xeno nucleic acid (XNA), or a combination thereof. In some embodiments, the polynucleotide is single-stranded. In some embodiments, the polynucleotide comprises a nucleic acid aptamer. As used herein, “aptamer’ refers to a sequence of DNA, RNA, XNA that binds a specific target, e.g., a biomolecule or a family of biomolecules. For further description of nucleic acid aptamers, see, for example, Bohrmann et al. (2022).

Embodiments of a method of synthesizing a CuAAC-accelerating ligand comprising a polynucleotide are disclosed herein. In some embodiments, a method of synthesizing includes preparing a CuAAC reaction mixture comprising an azide-labeled polynucleotide. In some embodiments, the azide-labeled polynucleotide is commercially available. In some embodiments, the azide-labeled polynucleotide is synthesized by using a transferase enzyme (e.g., a terminal deoxynucleotidyl transferase (TdT)) to add one or more azido-nucleotides (e.g., azido-deoxyuridine treiphosphate (azido dUTP), e.g., 5-(15-Azido-4,7,10,13-tetraoxa-pentadecanoyl-aminoallyl)-2′-deoxyuridine-5′-triphosphate, Triethylammonium salt (azide-PEG4-aminoallyl-dUTP)) to an oligomer (e.g., a 15-mer). The presence of one or more azide groups in the polynucleotide enables CuAAC ligation of the polynucleotide to one or more other molecules bearing an alkyne.

Disclosed herein are embodiments of a method of ligating a payload to a target via CuAAC in the presence of a CuAAC-accelerating ligand comprising a polynucleotide. In some embodiments, the target comprises an alkyne-derivatized thymidine analog (e.g., 5-ethynyl-2′-deoxyuridine (EdU)), thereby enabling detection of DNA (e.g., nascent DNA) in cells. In some embodiments, the target comprises an alkyne-derivatized uridine analog (e.g., 5-ethynyl uridine (EU)).

As used herein, the term “nascent” refers to a nucleic acid molecule that has been newly synthesized or is in the process of being synthesized. When cells are contacted with molecules (e.g., targets) such as an alkyne-derivatized thymidine analog or an alkyne-derivatized uridine analog, such molecules may be incorporated into (i.e., may metabolically label) nascent DNA or RNA, respectively, as it is being synthesized.

Disclosed herein are embodiments of a method of ligating a payload to a target via CuAAC in the presence of a CuAAC-accelerating ligand comprising a polynucleotide. In some embodiments, the method further comprises performing CuAAC in the presence of a template comprising a nucleic acid (i.e., a nucleic acid template). As used herein, the terms “template” and “splint” are used interchangeably.

As used herein, the phrase “nucleic acid template-driven proximity ligation” refers to CuAAC performed in the presence of a) a CuAAC-accelerating ligand comprising a polynucleotide and b) a template comprising a nucleic acid. In some embodiments, a template is complementary to the polynucleotide (i.e., the polynucleotide of a CuAAC-accelerating ligand comprising a polynucleotide). In some embodiments, a) the payload and/or the target comprise a polynucleotide; and b) the template is complementary to the payload and/or the target. As used herein, the term “complementary” refers to two or more polynucleotide sequences that are able to base pair with a reasonable expectation of specificity, i.e., that the two or more complementary polynucleotide sequences will preferentially form base pairs with each other rather than other (e.g., random) polynucleotide sequences. It is well known in the art that polynucleotide sequences about 20 nucleotides in length and shorter (e.g., about 18 nucleotides, about 15 nucleotides) are able to specifically base pair with complementary nucleotide sequences.

### Cells and Cell Culture

As used herein, a “population” of cells is any number of cells greater than 1, such as at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the terms “cell culture” and “tissue culture” are used interchangeably. Non-limiting examples of cells or cell lines grown in cell culture include Hela cells (e.g. American Type Culture Collection (ATCC) HeLa/CRM-CCL-2 cells), Chinese hamster ovary (CHO) cells (e.g., ATCC CHO-K1/CCL-61 cells), CHO-GFP-M20 cells (e.g., transgenic CHO cells harboring 32 tandem copies of the probe-binding sequence M20), and Jurkat cells (e.g., ATCC TIB-152 cells).

As used herein, the term “organoid” or refers to a cell cluster or aggregate that resembles an organ, or part of an organ, and possesses cell types relevant to that particular organ. Alternatively, “organoid” refers to a model of an organ or tissue, e.g., a simplified and/or miniaturized version of the organ or tissue that recapitulates characteristics of the organ or tissue in a state that is undeveloped, developing, fully developed, mature, aging, healthy, injured, diseased, and/or at risk for disease. An organoid can be a model of any stage organ development.

“Plating” refers to the action of dispensing cells (i.e., singularized cells or small aggregates of cells) onto/into a plate. As used herein, “seeding” refers to plating a given number of cells with the intention of culturing said cells to divide and produce a larger number of cells.

The terms “media” and “medium” may be used interchangeably herein when in reference to cell culture. A medium may comprise one or more supplements, such as a small molecule, an amino acid, a protein, a serum, a nutrient mixture, and the like, or a combination thereof. In some embodiments, a disclosed supplement may be replaced by a “suitable substitute.” In some embodiments, a suitable substitute comprises a supplement or combination of supplements that is different from but equivalent to the disclosed supplement, i.e. a supplement that exhibits the same or a similar biological effect as the disclosed supplement. Non-limiting examples of cell culture media include Dulbecco's Modified Eagle's Medium (DMEM) and Roswell Park Memorial Institute (RPMI 1640) medium. A non-limiting example of a cell culture medium supplement comprises fetal bovine serum (FBS). Non-limiting examples of cell culture conditions include incubation at 37° C., incubation in 5% CO₂, incubation in a water-saturated environment (i.e., at 100% relative humidity), or combinations of the foregoing.

In some embodiments, cell viability is quantified by a trypan blue dye exclusion method (see, for example, Strober (2015)).

### Methods of Intracellular Delivery of a CuAAC-Accelerating Ligand

### Electroporation

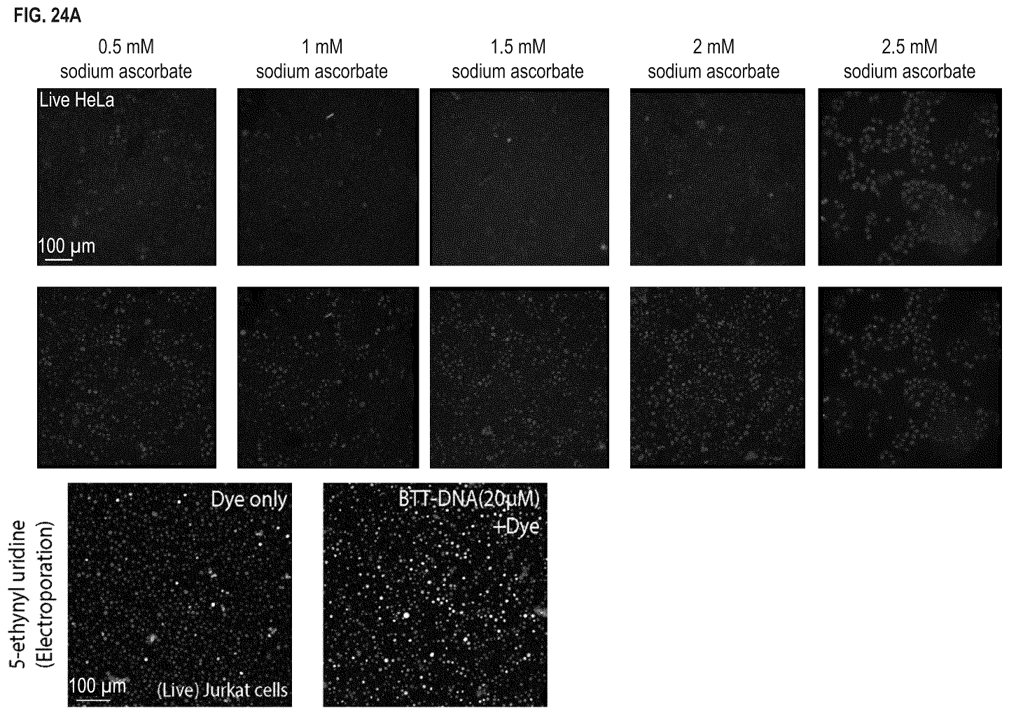

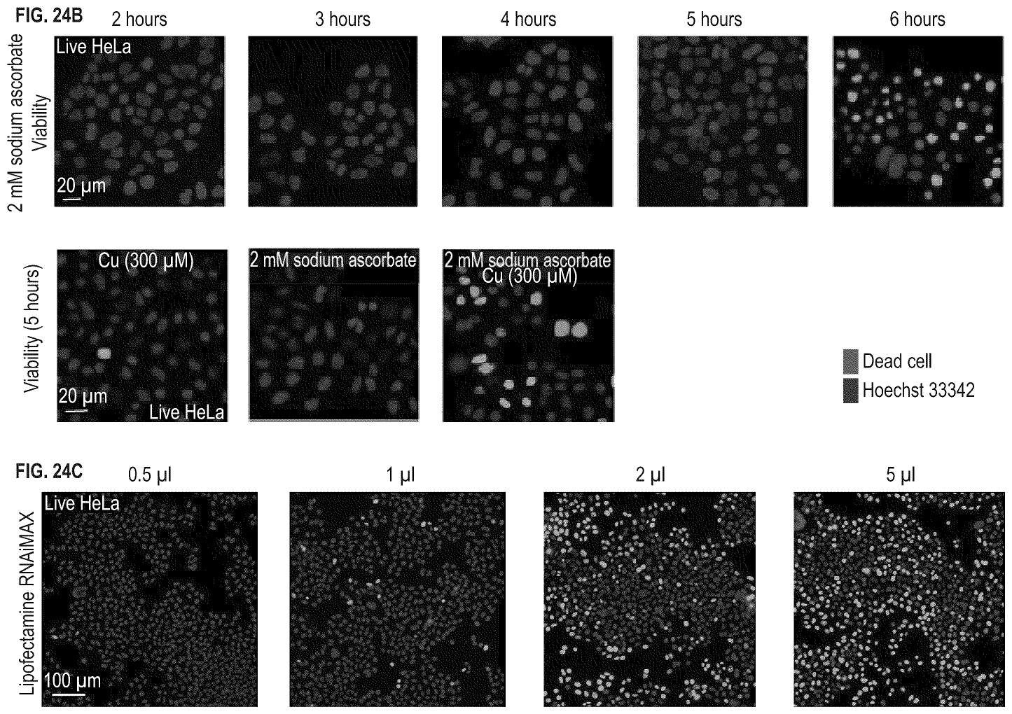

In some embodiments, intracellular delivery of a CuAAC-accelerating ligand (e.g., BTT_{(1,2)}-DNA, BTT-DNA) can be facilitated by electroporation (see FIG. 24A).

### Liposomal Intracellular Delivery of BTT-DNA

In some embodiments, a method for encapsulating the ligand in a liposome for intracellular delivery is described. Briefly, a major advance of some embodiments is to complex the oligonucleotide attachment of the ligand with an additional oligonucleotide, thereby producing a double-stranded oligonucleotide complex to achieve sufficient negative charge and encapsulate using commercial liposome formulations. This provides uniform delivery of the ligand with little to no toxicity. Example applications include intracellular labeling of biomolecules using CuAAC and drug discovery.

The DNA oligo attachment of BTT-DNA serves several purposes, including enabling the liposome encapsulation and delivery of the ligand into live cells. DNA provides a convenient method for intracellular delivery to encapsulate the copper within a liposome. LIPOFECTAMINE® (e.g., RNAIMAX®) is an example of a commercial liposome formulation that can be used to encapsulate BTT-DNA for intracellular delivery.

### Pore Forming Toxins for Intracellular Delivery of CuAAC Ligands in Live Cells

One of the significant challenges for intracellular delivery and intracellular click chemistry of biomolecules in live cells has been delivering the necessary CuAAC reagents inside the cell while avoiding trapping the reagents in the exosome. The present disclosure includes methods for delivery of the reagents using reversible, pore-forming toxins, e.g., Streptolysin-O (SLO), to achieve live cell CuAAC labeling. Example advantages include delivery of all reagents into the cytoplasm and some to the nucleus, avoiding endosomal delivery where it can get trapped, and use in tandem with liposome delivery methods to achieve even greater labeling efficiency. Example applications include a biosensor for live cell tracking of biomolecules and drugs and tracking the rate of drug metabolism. For example, SLO was used in intracellular click reactions in live cells to label RNA (see Example 7) and proteins (see Example 9).

### Pharmaceutical Compositions and Methods

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual.

In some embodiments, an agent is in a form of a pharmaceutical composition, or a pharmaceutically acceptable salt thereof. A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a pharmaceutical composition disclosed herein is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods disclosed herein further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linolcyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents disclosed herein may be prepared in accordance with standard procedures and are administered at dosages that are selected to reduce, prevent, or eliminate, or to slow or halt progression of, a condition being treated (Sec, e.g., Remington's Pharmaceutical Sciences, Mack Publishing Company, Easton, PA, and Goodman and Gilman's The Pharmaceutical Basis of Therapeutics, McGraw-Hill, New York, N. Y., the contents of which are incorporated herein by reference, for a general description of methods for administering various agents for human therapy).

In some embodiments, an agent disclosed herein is delivered using controlled or sustained-release delivery systems (e.g., capsules, biodegradable matrices). Example delayed-release delivery systems for drug delivery that would be suitable for administration of a composition described herein are described in U.S. Pat. No. 5,990,092 (issued to Walsh); 5,039,660 (issued to Leonard); 4,452,775 (issued to Kent); and 3,854,480 (issued to Zaffaroni), the entire teachings of which are incorporated herein by reference. Example biodegradable matrices are discussed in Li et al. (2020).

For oral administration, an agent may be in the form of, for example, a tablet, capsule, suspension or liquid. An agent is preferably made in the form of a dosage unit containing a therapeutically effective amount of an active ingredient. Examples of such dosage units are tablets and capsules. For therapeutic purposes, tablets and capsules can contain, in addition to an active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

Administration of the agent to the subject can be by parenteral or non-parenteral means. In some embodiments, an agent disclosed herein is administered intravenously, intra-arterially, intrathecally, intraventricularly, intramuscularly, intradermally, subcutaneously, intracranially, or spinally. “Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including an act of prescribing a drug or directing a subject to consume an agent. For example, as used herein, one (e.g., a physician) who instructs a subject (e.g., a patient) to self-administer an agent (e.g., a drug), or to have an agent administered by another and/or who provides a patient with a prescription for a drug is administering an agent to a subject. Administration of an agent can be once in a day or more than once in a day (e.g., twice a day or more). Administration of the agent can be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose.

In some embodiments, an agent disclosed herein is delivered systemically, such as via intravenous or subcutaneous injection. In some embodiments, the agent is delivered using an approach that enhances bioavailability in the target tissue or organ after administration. These approaches can include modification of the sugars or phosphate linkages, delivering as a duplex with a ligand-conjugated RNA molecule, formulation into an artificial exosome, liposome, polymer nanoparticle or lipid nanoparticle, or conjugation to lipids, antibodies, peptides, sugars, neuroactive molecules, or other moieties that enhance delivery to the target tissue or organ. Other methods of enhancing bioavailability in the target tissue or organ after administration will be known to one skilled in the art.

In some embodiments, a method disclosed herein comprises administering to the subject two or more agents, for example, 2, 3, 4, or 5 or more agents. In some embodiments, the two or more agents are administered together. In other embodiments, the two or more agents are administered separately, e.g., sequentially. In some embodiments, two or more agents are administered in the same composition. In some embodiments, two or more agents are administered in different compositions.

### EXEMPLIFICATION

### Example 1. General Materials and Methods

### Tissue Culture Conditions.

HeLa cells were grown in Dulbecco's Modified Eagle's Medium (DMEM) supplemented with 10% fetal bovine serum (FBS). Jurkat cells were grown in Roswell Park Memorial Institute (RPMI 1640) medium containing 10% FBS. All cells were incubated in a 5% CO₂, water-saturated incubator at 37° C.

### Epifluorescent Microscopy of Cultured Cells.

For HeLa cells, microscopy was performed using a Nikon inverted research microscope (Eclipse Ti2-E/Ti2-E/B) with a Plan Apo λ 20×/0.75 objective or Plan Apo λ 60×/1.40 oil objective (Nikon, Tokyo, Japan). An Epi-fi LED illuminator linked to the microscope assured illumination and controlled respective brightness of four types of LEDs of different wavelengths. Images were acquired using the Neutral Density (ND16) filter for labeling with CalFluor 647 azide. Images were acquired and processed using ImageJ and were shown as a single z-plane. Images acquired using the Neutral Density (ND16) filter were false-colored gray.

TABLE 1
Oligonucleotide sequences.

| SEQ | | | | |
|---|---|---|---|---|
| ID | | | | |
| NO. | Oligonucleotide | Use | 5′ Sequence | Modification |
| 1 | M20-right_3′azide | Ligand | GGTGCTCTTCGTCCA | 3′ Azide |
| | | synthesis | | |
| 2 | Random_M20_3′azide | Ligand | GCAGAGACACTATTG | 3′ Azide |
| | | synthesis | | |
| 3 | M20-alkyne-left | 5′ alkyne | CAAACACAACTCCTG | 5′ Hexynyl |
| | | DNA probe | | |
| 4 | M20_left_alkyne_20 | 5′ alkyne | CAAACACAACTCCTGGTC | 5′ Hexynyl |
| | | DNA probe | GA | |
| 5 | M20_left_alkyne_20-1 | 5′ alkyne | AAACACAACTCCTGGTCG | 5′ Hexynyl |
| | | DNA probe | AG | |
| 6 | M20_left_alkyne_20-2 | 5′ alkyne | AACACAACTCCTGGTCGA | 5′ Hexynyl |
| | | DNA probe | GG | |
| 7 | M20_left_alkyne_20_t | 5′ alkyne | TCAAACACAACTCCTGGT | 5′ Hexynyl |
| | | DNA probe | CGA | |
| 8 | M20_left_alkyne_20_ | 5′ alkyne | TTCAAACACAACTCCTGG | 5′ Hexynyl |
| | 2t | DNA probe | TCGA | |
| 9 | M20_left_alkyne_20_ | 5′ alkyne | TTTCAAACACAACTCCTG | 5′ Hexynyl |
| | 3t | DNA probe | GTCGA | |
| 10 | M20_left_alkyne_20_ | 5′ alkyne | TTTTCAAACACAACTCCT | 5′ Hexynyl |
| | 4t | DNA probe | GGTCGA | |
| 11 | DNAX1 splint | Template | CGACCTCGACCAGGAGTT | |
| | | | GTGTTTGTGGACGAAGAG | |
| | | | CACC | |
| 12 | DNAX2 splint | Template | CGACCTCGACCAGGAGTT | |
| | | | GTGTTTGTGGACGAAGAG | |
| | | | CACCAAACGACCTCGACC | |
| | | | AGGAGTTGTGTTTGTGGA | |
| | | | CGAAGAGCACC | |
| 13 | DNAX3 splint | Template | CGACCTCGACCAGGAGTT | |
| | | | GTGTTTGTGGACGAAGAG | |
| | | | CACCAAACGACCTCGACC | |
| | | | AGGAGTTGTGTTTGTGGA | |
| | | | CGAAGAGCACCAAACGAC | |
| | | | CTCGACCAGGAGTTGTGT | |
| | | | TTGTGGACGAAGAGCACC | |
| 14 | RNAX1 splint | Template | CGACCUCGACCAGGAGUU | |
| | | | GUGUUUGUGGACGAAGAG | |
| | | | CACC | |
| 15 | M20-right_3′azide- | | TGGACGAAGAGCACC | |
| | complementary | | | |

Quantification of Labeling Intensity of Biomolecules with BTT-DNA-Assisted CuAAC in on Fixed Cells.

Fluorescence intensities were obtained from random points in various raw fluorescence micrographs. A minimum of 175 points were acquired for analysis of EdU labeling, a minimum of 90 points were acquired for EU labeling, a minimum of 115 points were obtained for L-HPG labeling, a minimum of 85 points were acquired for Ac₄ManNAl labeling, and a minimum of 95 points were obtained for propargyl choline labeling. Intensity was normalized to the fluorescence intensity from no ligand control.

Quantification of Extracellular Labeling Intensity of Biomolecules with BTT-DNA-Assisted CuAAC on Live Cells.

Fluorescence intensities were obtained from random cells in various raw fluorescence micrographs. 5 random points were acquired from each cell membrane to obtain the average fluorescence of each cell. 60 cells were acquired for Ac4ManNAl labeling, and 45 cells were obtained for propargyl choline labeling. Intensity was normalized to the fluorescence intensity from no ligand control.

### Example 2. Synthesis and Purification

### Enzymatic Production of N₃-Labeled Oligomer Probe.

A 15-unit (15-mer) single-stranded DNA (ssDNA) oligomer (5′-GGTGCTCTTCGTCCA-3′; SEQ ID NO:1) was obtained from Integrated DNA Technologies (IDT; Coralville, IA) and reconstituted to 100 μM with nuclease-free H₂O (NF—H₂O). A volume of 250 μl of 1 mM azide-PEG4-aminoallyl-dUTP (azido-UTP; Jena Bioscience, Jena, Germany), 100 μl of 10× transferase buffer, 100 μl of 100 μM 15-mer ssDNA oligomer, 30 μl of terminal deoxynucleotidyl transferase (TdT) enzyme (20,000 U/ml), 100 μl of 10× CoCl₂, and 420 μl of NF—H₂O were mixed in a total reaction volume of 1000 μl. The reaction was vortexed for several seconds then incubated at 37° C. for 90 minutes. The reaction was stopped by increasing the temperature to 70° C. for 10 minutes on a heat block. The product was purified by ethanol precipitation and the pellet was resuspended with 50 μl NF—H₂O, resulting in a pure 3′ azide (N₃)-labeled 15-mer ssDNA oligomer. The concentration of N₃-labeled 15-mer ssDNA oligomer was measured using the Qubit ssDNA Assay Kit (Thermo Fisher Scientific, Waltham, MA).

### Synthesis and Purification of BTT_{(1,2)}-DNA Ligand.

The commercially available BTTAA (2-(4-((Bis((1-(tert-butyl)-1H-1,2,3-triazol-4-yl)methyl)amino)methyl)-1H-1,2,3-triazol-1-yl) acetic acid; CAS No: 1334179-85-9) ligand was used to stabilize the copper (I) oxidation state and maintain the fast kinetics for copper-catalyzed azide-alkyne cycloaddition (CuAAC) in aqueous solution. To enhance sensitivity and reduce the total copper concentration required for CuAAC, the precursor molecule S1 (N,N-bis((1-tert-butyl-1H-1,2,3-triazol-4-yl)methyl) prop-2-yn-1-amine; CAS No. 1257633-68-3), which contains two tert-butyl groups with an alkyne, was used to synthesize a CuAAC-accelerating ligand, BTT_{(1,2)}-DNA (FIG. 1A) with a DNA scaffold. S1 was ligated to a single-strand DNA oligonucleotide probe bearing a single or double 3′ azide, added using TdT and azido-UTP, via CuAAC, to produce BTT_{(1,2)}-DNA (FIG. 1A).

Specifically, a 100-fold molar excess of N,N-bis((1-tert-butyl-1H-1,2,3-triazol-4-yl)methyl) prop-2-yn-1-amine (S1; purchased from the Albert Einstein College of Medicine (AECOM) Chemical Synthesis Core Facility) was reacted with 3′ N₃-labeled 15-mer ssDNA oligomer using BTTAA ligand-assisted CuAAC ([BTTAA]: [CuSO₄]=2:1). The reaction mixture was prepared by adding the following reagents: N_{3(1,2)}-DNA (50 M), S1 (20 mM), premixed copper sulfate (CuSO₄; 5 mM) and BTTAA (20 mM), then sodium ascorbate (100 mM) (Table 2). The reaction mixture was shaken at 600 revolutions per minute (rpm) for 1 hour at 37° C., resulting in a crude BTT_{(1,2)}-DNA ligand. The BTT_{(1,2)}-DNA ligand was purified using 3.5 kDa molecular weight cut-off (MWCO) dialysis tubing (Repligen, Waltham, MA) for 24 hours in 3.5 L of water at room temperature. The Invitrogen Qubit ssDNA Assay Kit was used to measure the concentration of the purified BTT_{(1,2)}-DNA ligand.

TABLE 2
BTT_{(1,2)}-DNA ligand synthesis reaction mixture.

| | Stock | | Volume | | Final | |
|---|---|---|---|---|---|---|
| Reagent | Concentration | | Added | | Concentration | |
| N3_{(1,2)}-DNA | 50 | μM | 96 | μl | 6 | μM |
| S1 alkyne | 20 | mM | 24 | μl | 600 | μM |
| CuSO₄ | 5 | mM | 12 | μl | 75 | μM |
| BTTAA | 20 | mM | 6 | μl | 150 | μM |
| Sodium Ascorbate | 100 | mM | 20 | μl | 2.5 | mM |
| NF-H₂0 | | | 642 | μl | | |
| Total volume | | | 800 | μl | | |

The synthesis of this ligand was confirmed using a tris/boric acid/EDTA (TBE)-Urea gel. The relative abundance of the single-BTT (BTT₍₁₎-DNA) and double-BTT (BTT₍₂₎-DNA) CuAAC-accelerating ligands, based on the incorporation of a single or double azido-UTP via TdT enzyme, is 47.8% and 52.2%, respectively (FIG. 1B). High-performance liquid chromatography (HPLC) and dialysis were performed to purify both populations of BTT_{(1,2)}-DNA ligand.

### Synthesis and Purification of BTT-DNA Ligand.

The precursor molecule S1, which contains two tert-butyl groups with an alkyne, was ligated to a single-stranded DNA oligonucleotide probe bearing a single 3′ azide via CuAAC to produce BTT-DNA (FIG. 2A).

Specifically, a 200-fold molar excess of S1 alkyne (S1; N,N-bis((1-tert-butyl-1H-1,2,3-triazol-4-yl)methyl) prop-2-yn-1-amine; purchased from the Albert Einstein College of Medicine (AECOM) Chemical Synthesis Core Facility) was reacted with 3′ N₃-labeled 15-mer ssDNA oligomer using BTTAA ligand-assisted CuAAC ([BTTAA]: [CuSO₄]=2:1). The reaction mixture was prepared by adding the following reagents: N₃-DNA (100 μM), S1 (40 mM), premixed copper sulfate (5 mM) and BTTAA (20 mM), then sodium ascorbate (100 mM) Table 3). The reaction mixture was then shaken at 600 rpm for 30 minutes at 37° C., resulting in a crude BTT-DNA ligand. The BTT-DNA ligand was purified using 3.5 kDa molecular weight cut-off (MWCO) dialysis tubing for 24 hours in 3.5 L of water at room temperature (Repligen). The Invitrogen Qubit ssDNA Assay Kit was used to measure the concentration of the purified BTT-DNA ligand.

TABLE 3
BTT-DNA ligand synthesis reaction mixture.

| | Stock | | Volume | | Final | |
|---|---|---|---|---|---|---|
| Reagent | Concentration | | Added | | Concentration | |
| N₃-DNA | 100 | μM | 48 | μl | 6 | μM |
| S1 alkyne | 40 | mM | 24 | μl | 1200 | μM |
| CuSO₄ | 5 | mM | 12 | μl | 75 | μM |
| BTTAA | 20 | mM | 6 | μl | 150 | μM |
| Sodium Ascorbate | 100 | mM | 20 | μl | 2.5 | mM |
| NF-H₂0 | | | 690 | μl | | |
| Total volume | | | 800 | μl | | |

The synthesis of this ligand was confirmed using a TBE-Urea gel (FIG. 3A). BTT-DNA ligand was then purified with dialysis.

### Example 3. Characterization

### Inductively Coupled Plasma Mass Spectrometry (ICP-MS).

To assess the quantity of copper that was complexed with the BTT_{(1,2)}-DNA ligand and the BTT-DNA ligand, the ligands were prepared for ICP-MS. The sample (0.1 ml) was dissolved in 0.9 ml of concentrated HNO₃. The sample was heated for an hour at 90° C. Then the temperature was increased to 150° C., at which the sample was boiled. During the boiling period, 0.5 ml of concentrated HNO₃ was added to the sample twice (a total of 1 ml concentrated HNO₃ was added during boiling). When the sample was completely dried, an additional 0.5 ml of concentrated HNO₃ was added. The sample was then diluted with NF—H₂O to bring the final volume to 10 ml. To confirm the accuracy of the ICP-MS data, two technical replicates were performed. Samples were submitted to Robertson Microlit Laboratories (Ledgewood, New Jersey) for ICP-MS.

### Characterization of BTT_{(1,2)}-DNA Ligand.

The relative efficacy of each version of the CuAAC-accelerating ligand (BTT₍₁₎-DNA and BTT₍₂₎-DNA) to catalyze the formation of a ligation product via CuAAC was tested. An alkyne-labeled DNA oligonucleotide was ligated to 3-Azido-7-hydroxycoumarin (a fluorogenic azide dye; CAS No: 817638-68-9) in the presence of sodium ascorbate and BTT₍₁₎-DNA or BTT₍₂₎-DNA ligand. The fold change of the BTT₍₁₎-DNA ligand was 11.10 and that of the BTT₍₂₎-DNA ligand was 11.28, indicating that the two ligands performed similarly (FIG. 1C).

The mixed population of BTT_{(1,2)}-DNA ligand was confirmed to have similar efficacy after purification by HPLC and dialysis or by dialysis alone. To lower the sample loss due to HPLC and accelerate the purification process, synthesis products were therefore purified directly with dialysis for subsequent experiments, allowing the two populations of BTT_{(1,2)}-DNA ligand to remain mixed.

Pilot CuAAC studies using the BTT_{(1,2)}-DNA ligand suggested the formation of ligation product in the absence of free copper (data not shown), so inductively coupled plasma mass spectrometry (ICP-MS) was performed to assess the presence of copper in the purified ligand. Surprisingly, the ratio of copper to BTT_{(1,2)}-DNA was 7.5:1, even after dialysis (FIG. 1D).

### Characterization of BTT-DNA Ligand.

Using inductively coupled plasma mass spectrometry (ICP-MS), the presence of copper in the purified BTT-DNA ligand was assessed. The ratio of copper to BTT-DNA was 10:1 (FIG. 2A). The same azide-modified DNA that was subjected to the same reaction conditions without the presence of S1 showed a ratio of copper to DNA of 7:1, indicating that the DNA as well as the triazole groups chelated the copper.

### Example 4. Reaction Kinetics

### Reaction Kinetics of BTT_{(1,2)}-DNA Ligand.

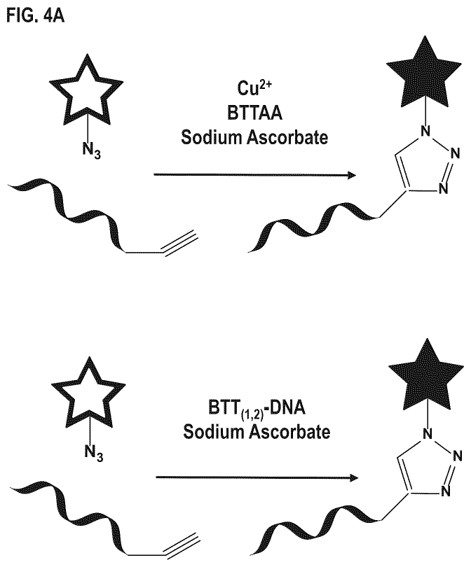

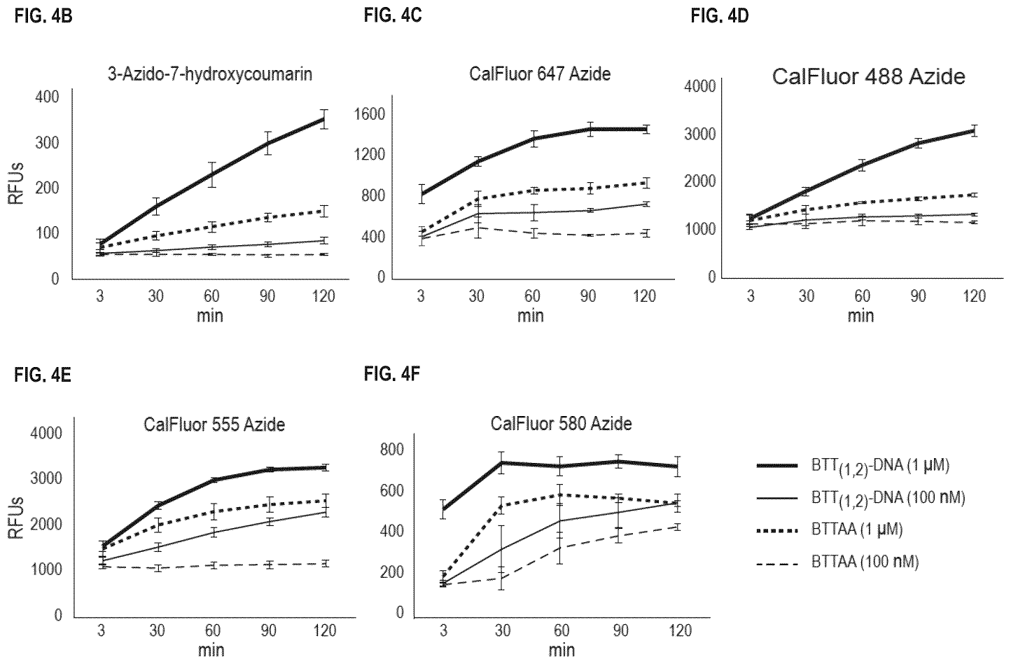

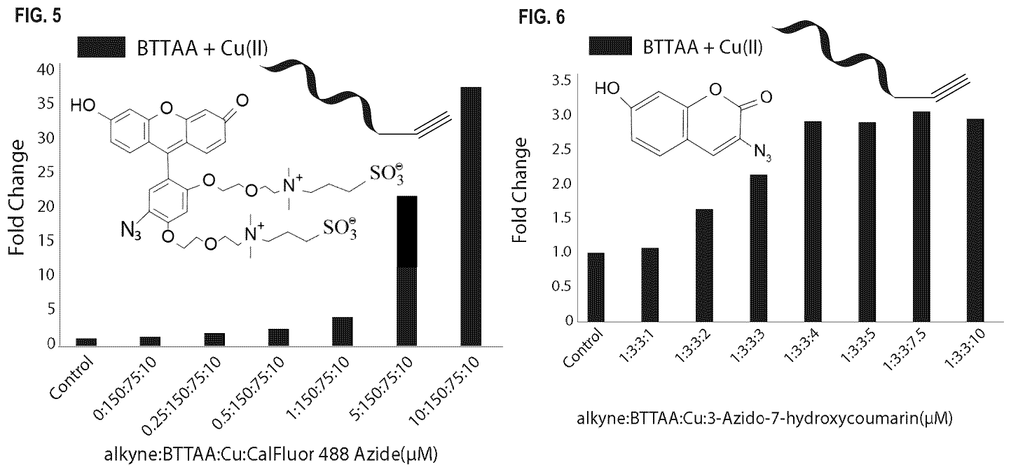

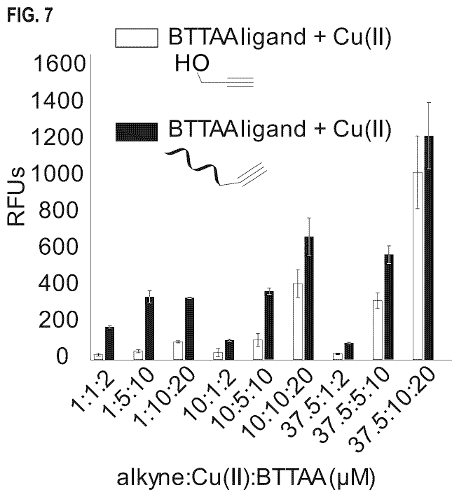

The relative reactivity of Cu(I) catalysts in the form of BTTAA-Cu(I) and the new Cu(I) accelerating ligand, BTT_{(1,2)}-DNA ligand (which was complexed with an average of 7.5 copper molecules) was determined in a fluorogenic plate-reader assay by reacting a 15-mer DNA oligomer bearing a 5′ alkyne moiety (henceforth referred to as 5′ alkyne DNA) with a series of azido fluorogenic dyes²⁴ using CuAAC (FIG. 4A). To assess the reactivity of the Cu(I) catalyst in complex with BTTAA and the BTT_{(1,2)}-DNA ligand, the reaction was optimized (FIGS. 5-7) and the 5′ alkyne DNA was ligated to a series of fluorogenic dyes using CuAAC with the fluorescence measured over 2 hours (FIGS. 4B-4F).

The click reaction plate assay (fluorogenic plate-reader assay) reaction mixture was prepared by adding the following reagents: phosphate-buffered saline (PBS; 10×), alkyne (10 μM), premixed copper sulfate (5 μM) and BTTAA (10 μM) or BTT_{(1,2)}-DNA (10 μM), azido dye (75 μM), then sodium ascorbate (25 mM). The click reaction mixture was added to the wells (20 μL/well) of a 384-well fluorescence plate and the click reaction was carried out for 2 hours at room temperature. The fluorescent alkyne-azide cyclo-adduct was detected using SPECTRAMAX® Gemini EM plate reader with excitation/emission wavelengths respectively at 404/477 nm for 3-Azido-7-hydroxycoumarin, 500/521 nm for CalFluor 488 azide, 561/583 nm for CalFluor 555 azide, 591/609 nm for CalFluor 580 azide, and 657/674 nm for CalFluor 647 azide.

TABLE 4
Click reaction plate assay for BTT_{(1,2)}-DNA.

| | | | Volume | | Final | |
|---|---|---|---|---|---|---|
| Reagent | Stock | | added | | concentration | |
| PBS | 10X | | 2 | μl | 1X | |
| Alkyne | 10 | μM | 2 | μl | 1 | μM |
| BTT_{(1,2)}-DNA (or Cu and BTTAA | 10 | μM | 2 | μl | 1 | μM |
| premix) | | | | | | |
| Azido dye | 75 | μM | 2 | μl | 7.5 | μM |
| NF- H₂O | | | 10 | μl | | |
| Sodium Ascorbate | 25 | mM | 2 | μl | 2.5 | mM |
| Total volume | | | 20 | μl | | |

BTT_{(1,2)}-DNA consistently showed higher activity in accelerating the CuAAC reaction than BTTAA for all dyes tested. For the CuAAC reactions that were assisted by 100 nM BTT_{(1,2)}-DNA ligand, an increase in fluorescence intensity from 59.46 to 87.68 relative fluorescence units (RFUs) was observed over a 2 hour reaction. For the reactions that were assisted by 1 μM BTT_{(1,2)}-DNA ligand, fluorescence intensity increased from 79.64 to 355.82 RFUs over 2 hours. At 100 nM ligand concentration, the BTT_{(1,2)}-DNA ligand outperformed the commercial ligand 1.56-fold, demonstrating that BTT_{(1,2)}-DNA is an effective ligand in the nanomolar range and in the absence of free Cu(I). The difference was even more striking at 1 μM BTT_{(1,2)}-DNA, at which the fluorescence produced was 2.33-fold higher than commercial ligand at 2 hours (FIG. 4B). Upon click reaction in the presence of BTT_{(1,2)}-DNA ligand, the fluorescence of the CalFluor 647 azide produced at 1 μM BTT_{(1,2)}-DNA was 1.56-fold higher than BTTAA ligand at 2 hours; the BTT_{(1,2)}-DNA ligand also outperformed the BTTAA ligand 1.63-fold at the concentration of 100 nM (FIG. 4C). The CalFluor 488 azide showed a significant difference at 1 μM BTT_{(1,2)}-DNA ligand concentration—the fluorescence produced was 1.77-fold higher than that produced by the commercial ligand at 2 hours, while the difference between the BTT_{(1,2)}-DNA and BTTAA ligand at the concentration of 100 nM was negligible (FIG. 4D). Surprisingly, CalFluor 555 azide showed a significant difference between the BTT_{(1,2)}-DNA and BTTAA ligand at 100 nM of the ligand as a 1.96-fold increase was achieved (FIG. 4E). While CalFluor 580 had the lowest reaction yield among the CalFluor azide dyes in the 100 nM to 1 μM concentration range of ligand, and the pattern differed from the other three CalFluor azide dyes, a 1.26-fold increase and a 1.32-fold increase in fluorescent signal were produced from the BTT_{(1,2)}-DNA ligand-accelerated reaction as compared to the commercial ligand at 100 nM and 1 μM of ligand, respectively (FIG. 4F).

### Reaction Kinetics of BTT-DNA Ligand.

The reactivity of the CuAAC reaction in the presence of the new Cu(I) accelerating ligand, BTT-DNA ligand, was determined using a fluorogenic plate-reader assay. A 15-mer DNA oligomer bearing a 5′ alkyne moiety (henceforth referred to as 5′ alkyne DNA) was reacted to a series of azido fluorogenic dyes²⁴ using CuAAC (FIGS. 2B, 2C). To assess the reactivity of the Cu(I) catalyst in complex with the BTT-DNA and BTTAA ligands, the reaction was optimized (FIGS. 3B-3C, 5-7), and the 5′ alkyne DNA was ligated to two different fluorogenic dyes using CuAAC with the fluorescence measured over 2 and 4 hours, respectively (FIGS. 2D-2I).

The click reaction mixture for the fluorogenic plate-reader assay was prepared by adding the following reagents: PBS (10×), alkyne (10 μM), premixed copper sulfate (5 μM) and BTTAA (10 μM) or BTT-DNA (10 μM), and azido dye (75 μM), then sodium ascorbate (25 mM). The click reaction mixture was added to the wells (20 μL/well) of the 384-well fluorescence plate and the click reaction was carried out for 2 hours at room temperature. The fluorescent alkyne-azide cyclo-adduct was detected using SPECTRAMAX® Gemini EM plate reader with excitation/emission wavelengths respectively at 404/477 nm for 3-Azido-7-hydroxycoumarin, 500/521 nm for CalFluor 488 azide, 561/583 nm for CalFluor 555 azide, 591/609 nm for CalFluor 580 azide, 657/674 nm for CalFluor 647 azide.

TABLE 5
Click reaction plate assay for BTT-DNA.

| | | | Volume | | Final | |
|---|---|---|---|---|---|---|
| Reagent | Stock | | added | | concentration | |
| PBS | 10X | | 2 | μl | 1X | |
| Alkyne | 10 | μM | 2 | μl | 1 | μM |
| BTT-DNA (or Cu and BTTAA | 10 | μM | 2 | μl | 1 | μM |
| premix) | | | | | | |
| Azido dye | 75 | μM | 2 | μl | 7.5 | μM |
| NF- H₂O | | | 10 | μl | | |
| Sodium Ascorbate | 25 | mM | 2 | μl | 2.5 | mM |
| Total volume | | | 20 | μl | | |

BTT-DNA consistently increased activity in accelerating the CuAAC reaction over BTTAA activity for 3-Azido-7-hydroxycoumarin in the nanomolar range. For the CuAAC reactions assisted by 100 nM BTT-DNA ligand, an increase in fluorescence intensity from 6.83 to 125.77 RFUs over a 4-hour reaction was observed. For the reactions assisted by 500 nM BTT-DNA ligand, an increase in fluorescence intensity from 19.63 to 586.96 RFUs over a 4-hour reaction was observed. For the reactions assisted by 1000 nM BTT-DNA ligand, the fluorescence intensity increased from 90.41 to 701.43 RFUs over 4 hours. Even at 100 nM ligand concentration, the BTT-DNA ligand outperformed the commercial ligand 1.39-fold, demonstrating that BTT-DNA is an effective ligand in the nanomolar range and the absence of free Cu(I). The difference was even more striking at 1000 nM BTT-DNA, at which the fluorescence produced was 3.00-fold higher than the commercial ligand at 2 hours. Surprisingly, at 500 nM ligand concentration, the BTT-DNA ligand outperformed the commercial ligand 4.80-fold and produced an even higher fold-change at 1000 nM ligand concentration (FIG. 2F). The rate of CuAAC reaction that was assisted by BTT-DNA ligand was 2.5765×10⁻⁵ min⁻¹ nM⁻¹, demonstrating that the CuAAC reaction accelerated by BTT-DNA was significantly faster than that accelerated by commercial BTTAA ligand, for which the reaction rate was 5.046×10⁻⁶ min⁻¹ nM⁻¹ (FIG. 2G).

The fluorogenic plate-reader assay was also performed using CalFluor 488 azide. Upon click reaction in the presence of BTT-DNA ligand, the fluorescence of the CalFluor 488 azide produced at 1000 nM BTT-DNA was 2.53-fold higher than BTTAA ligand at 2 hours. The BTT-DNA ligand also outperformed the BTTAA ligand 7.54-fold at the concentration of 500 nM (FIG. 2H). The most prominent enhancement of the fluorescence was constantly achieved at 500 nM ligand concentration for both fluorogenic dyes. For the CalFluor 488 azide, the rate of CuAAC reaction that was assisted by BTT-DNA ligand was 4.38×10⁻⁵ min⁻¹ nM⁻¹, while the rate of CuAAC reaction assisted by commercial BTTAA ligand was 1.10×10⁻⁶ min⁻¹ nM⁻¹ (FIG. 2I).

### Contribution of DNA Sequence in Ligand Design

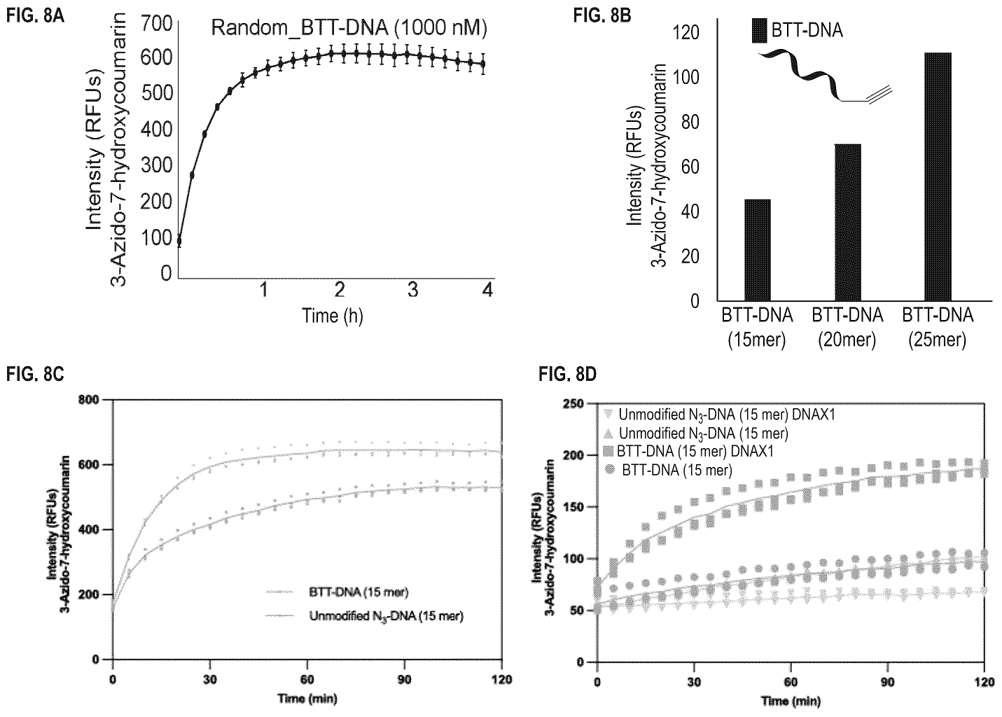

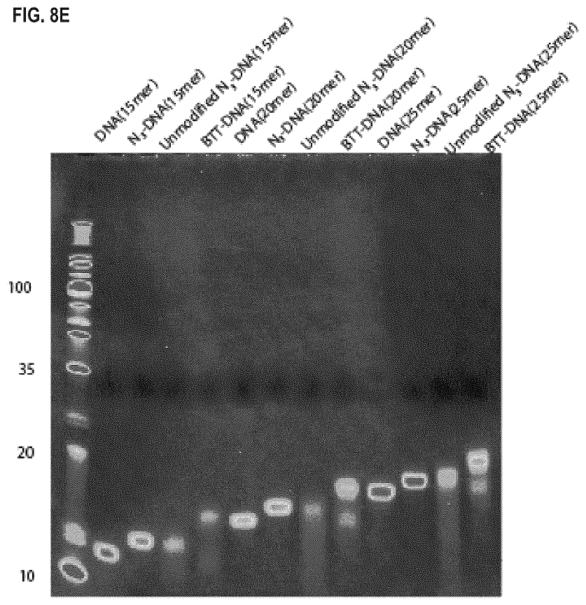

To assess the contribution of DNA sequence to the reactivity of the BTT-DNA ligand, the reaction was also evaluated with a scrambled (random) single-stranded DNA sequence (SEQ ID NO:2) using the fluorescent plate-reader assay. Identical activity was observed regardless of the sequence (FIG. 8A). Thus, the strategy to synthesize the new CuAAC-accelerating ligand can be applied to make a BTT-DNA ligand with any sequence.

Stability of BTT-DNA Ligand after Cold Storage

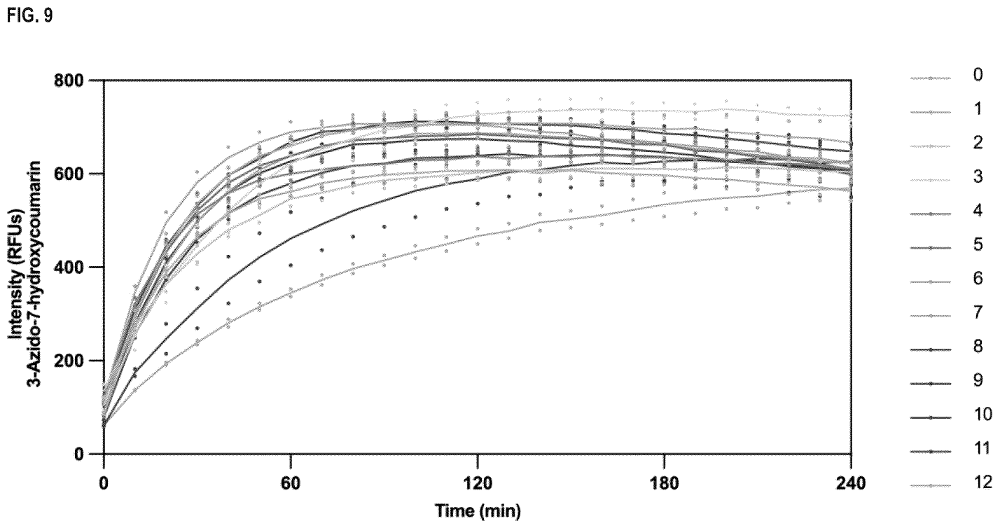

To evaluate the stability of the BTT-DNA ligand, the fluorogenic plate-reader assay was performed as described above using 3-Azido-7-hydroxycoumarin in the presence of the BTT-DNA ligand for 4 hours monthly for twelve months (FIG. 9). Using a concentration of 1000 nM BTT-DNA ligand, the reaction kinetics were unchanged. There was no significant difference between the fluorescence intensity of each sample across time points over twelve months, indicating that the BTT-DNA ligand can be stable for at least twelve months while stored at −20° C.

### Example 5. Proximity Ligation

DNA and RNA Template-Driven Proximity Ligation with BTT_{(1,2)}-DNA Ligand.

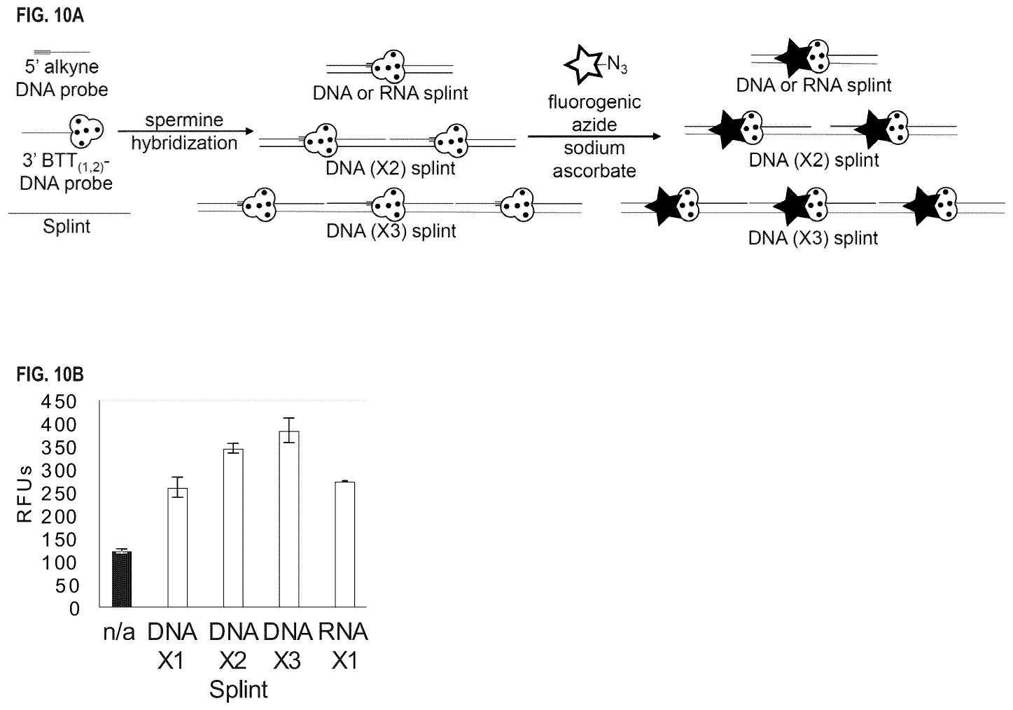

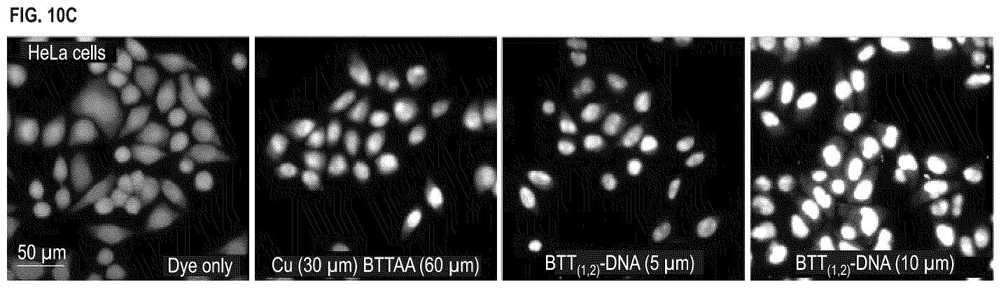

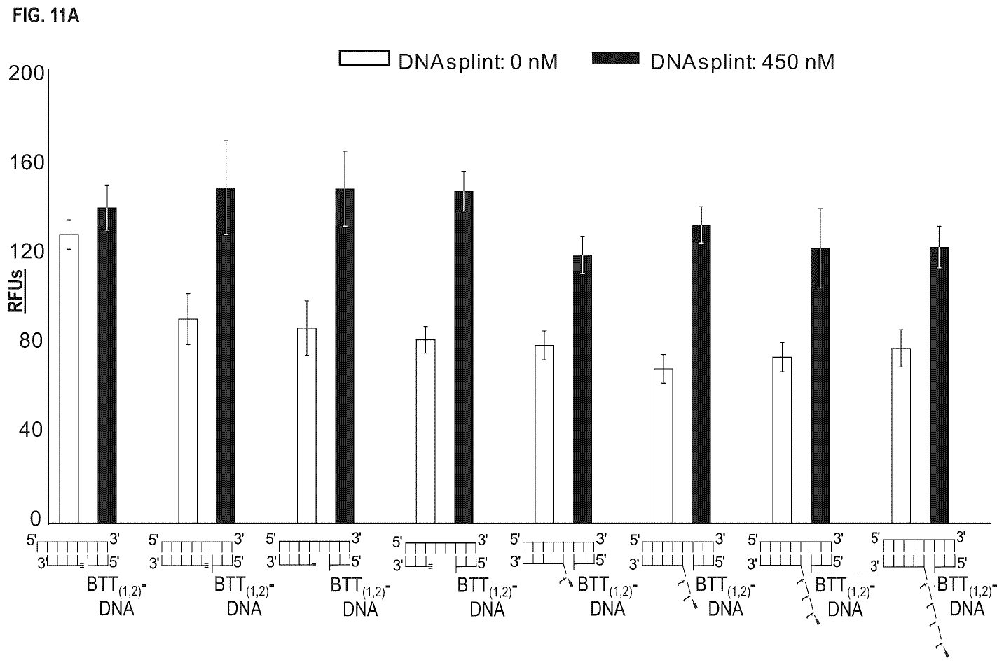

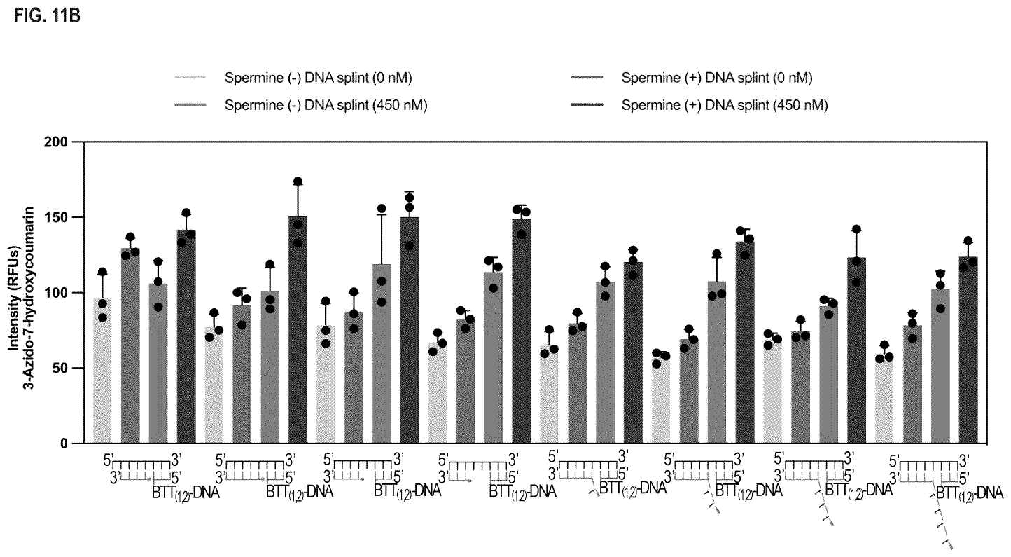

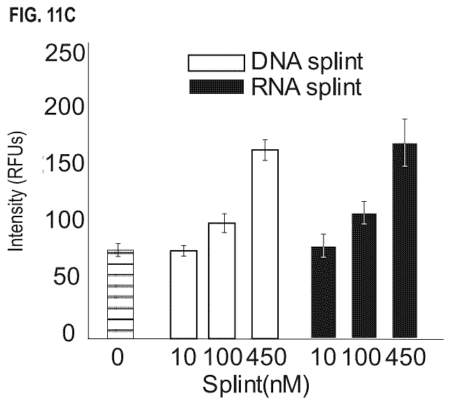

To increase the local concentration of the reaction components, a DNA splint (DNAX1 splint of SEQ ID NO:11) that was complementary to both the 5′ alkyne DNA probes (SEQ ID NOs: 3-10) and 3′ BTT_{(1,2)}-DNA ligand was used (FIG. 10A). The reaction was supplemented with spermine, a polyamine bearing multiple amino groups, to improve hybridization (FIG. 11A). The addition of both DNA and RNA (RNAX1 splint of SEQ ID NO: 14) splints enhanced the ligation of the fluorogenic azide with 5′ alkyne DNA (2.13-fold and 2.21-fold, respectively) facilitated by the BTT_{(1,2)}-DNA probe (FIG. 11C, FIG. 10A). To assess the appropriate distance to position the 5′ alkyne on the DNA probe to maximize ligation to the fluorogenic azide, the fluorogenic plate-reader assay was performed as described above (Table 4; for the DNA and RNA template-driven proximity ligation click reaction, DNA or RNA (0.45 μM) and spermine (1 mM) were added after BTT_{(1,2)}-DNA) for 8 different 5′ alkyne DNA probes. The DNA alkyne probe that had a 2-nucleotide space from the BTT_{(1,2)}-DNA probe was selected (FIG. 11B, black box). The splint concentration range which could drive the proximity ligation was evaluated. A splint concentration as low as 100 nM was able to drive the reaction (FIG. 11C). These data are consistent with results observed with purified BTT-DNA (data not shown).

To enhance the reaction kinetics, DNA splints harboring 2 and 3 repeats of the binding sequence (DNAX2 splint of SEQ ID NO:12; DNAX3 splint of SEQ ID NO: 13), which can double and triple the ligation, respectively, were designed (FIG. 10A). The absorbance after a 1-hour incubation time in fluorogenic plate-reader assay for each condition was assessed. The total fluorescence produced from a single DNA splint-enhanced reaction was 236.71±12.16 RFUs, which was 2.33-fold higher than the fluorescence produced from random collisions (FIG. 10B). Splints harboring more binding sites had higher fluorescence intensity. The absorbance increased from 236.71±12 RFUs to 310.98±7.11 RFUs and 344.10±9.82 RFUs when the binding sites doubled and tripled, respectively (FIG. 10B, FIG. 11).

DNA and RNA Template-Driven Proximity Ligation with BTT-DNA Ligand.

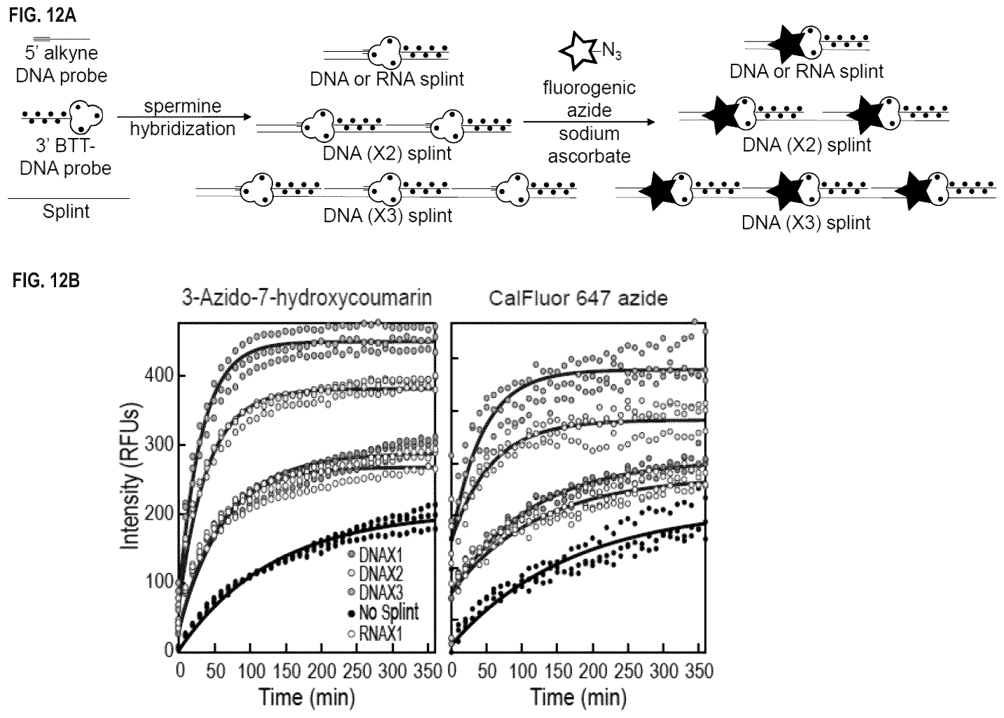

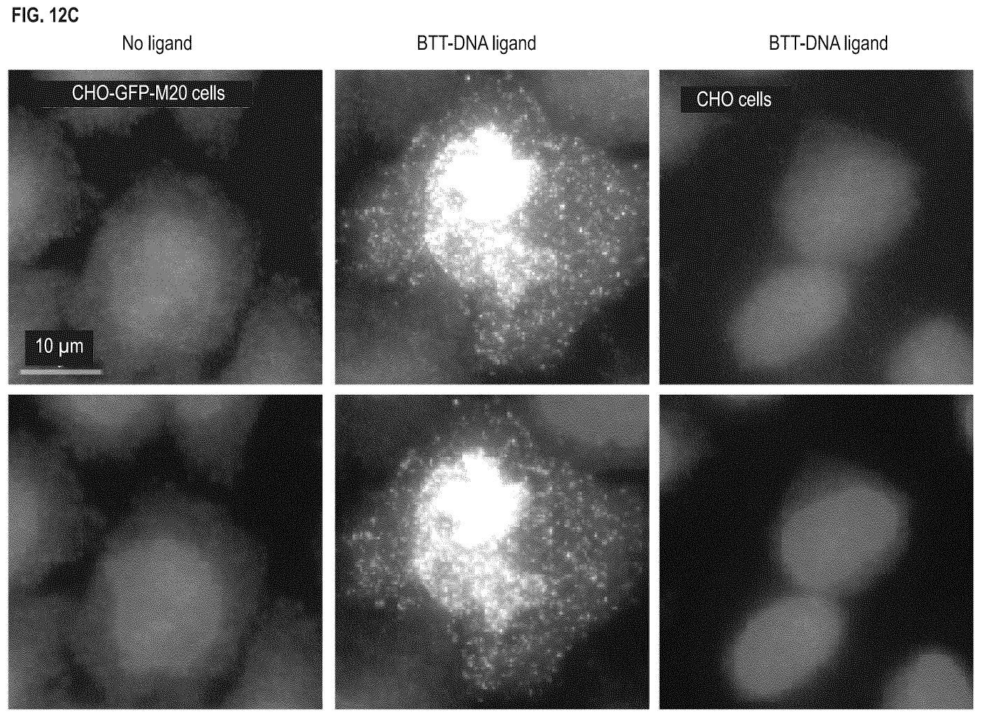

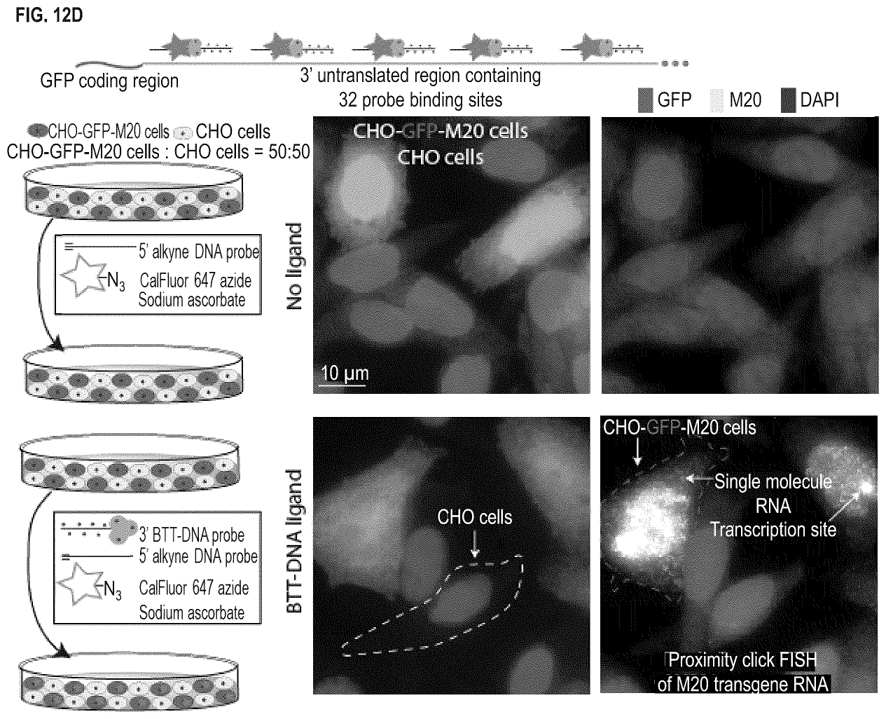

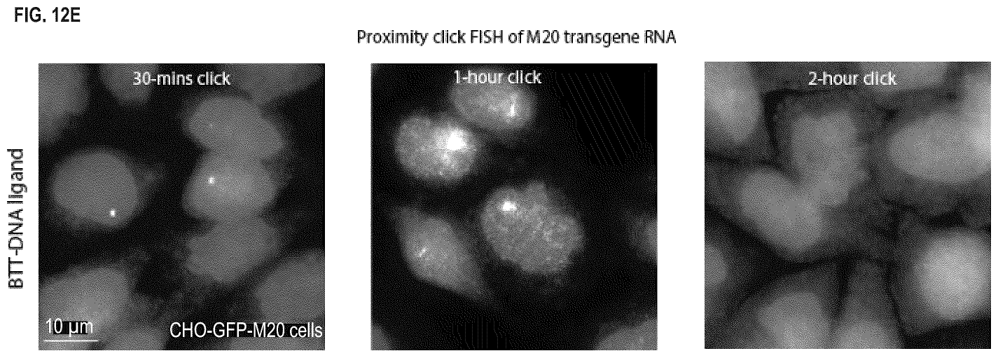

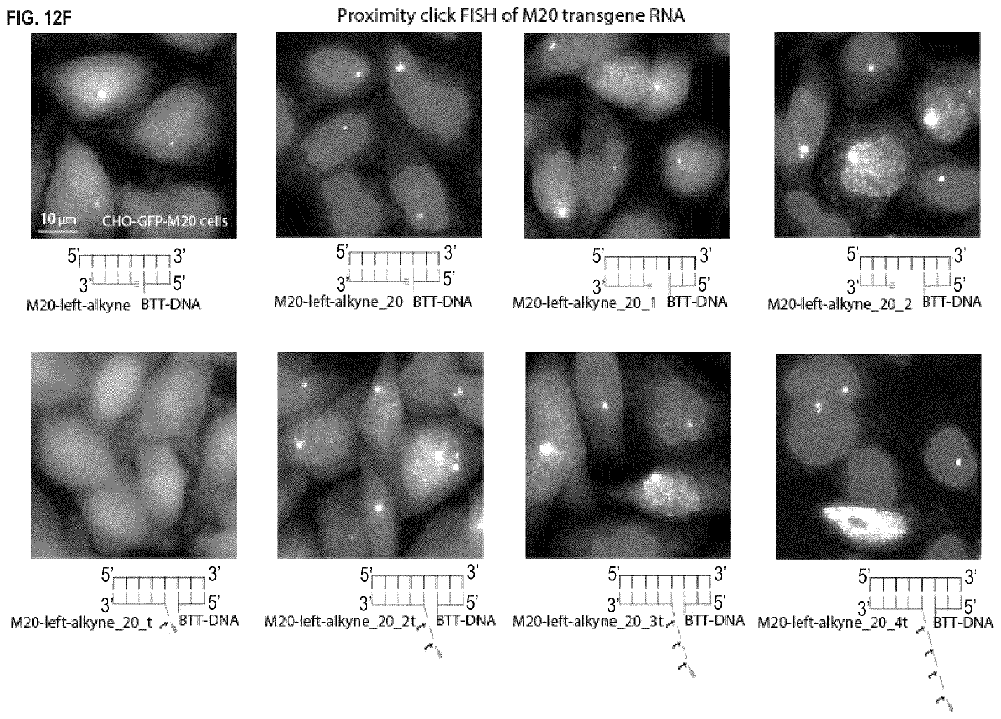

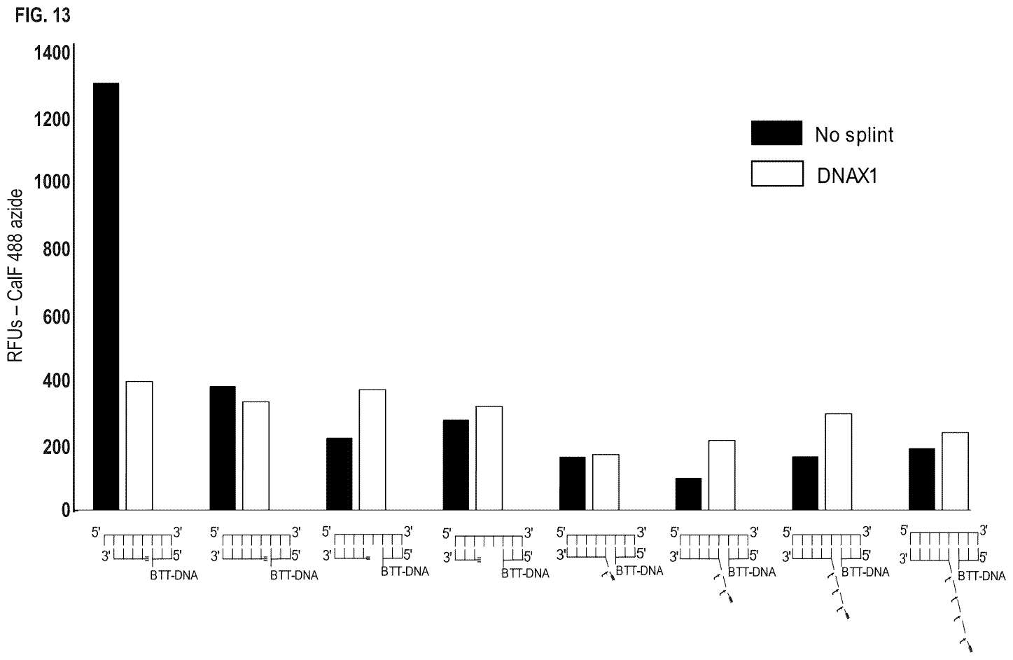

To increase the local concentration of the reaction components, a DNA splint (SEQ ID NO: 11) was designed to be complementary to both the 5′ alkyne DNA probes (SEQ ID NOS: 3-10) and 3′ BTT-DNA ligand (FIG. 12A). The reaction was supplemented with spermine, a polyamine bearing multiple amino groups, and NaCl to improve hybridization (FIG. 13). To assess the appropriate distance to position the 5′ alkyne on the DNA probe for maximum ligation to the fluorogenic azide, the fluorogenic plate-reader assay was performed (Table 5; for the DNA and RNA template-driven proximity ligation click reaction, DNA or RNA (4.5 μM), spermine (1 mM), and NaCl (4M) were added after BTT-DNA) for eight different 5′ alkyne DNA probes. The DNA alkyne probe with a one nucleotide distance from the BTT-DNA probe was selected (FIG. 13).

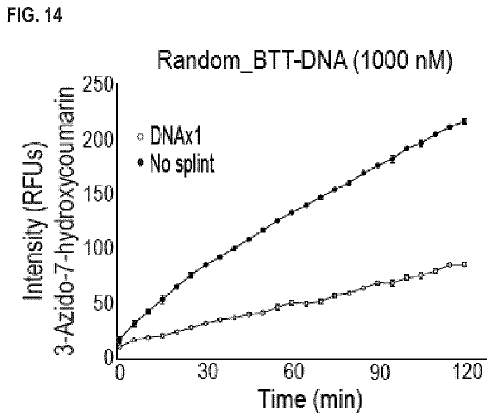

To enhance the reaction kinetics, DNA splints harboring 2 (SEQ ID NO:12) and 3 (SEQ ID NO: 13) repeats of the binding sequence, which can theoretically double and triple the ligation, were designed (FIG. 12A). The absorbance was assessed after a 6-hour incubation time using the fluorogenic plate-reader assay for each condition. For 3-Azido-7-hydroxycoumarin, the total fluorescence produced from a single DNA splint-enhanced reaction was 301.94 RFUs, which was 1.53-fold higher than the fluorescence produced from random collisions (i.e., no splint). As expected, no fluorescence was produced when substituting the targeted BTT-DNA strand with a scrambled BTT-DNA sequence that was not complementary to the DNA splint (FIG. 14). Splints harboring more binding sites had higher fluorescence intensity. The absorbance increased from 301.94 RFUs to 393.76 RFUs and 455.10 RFUs when the binding sites doubled and tripled, respectively (FIG. 12B). For the CalFluor 647 azide dye, the total fluorescence produced increased from 290.77 RFUs to 403.17 RFUs, 491.40 RFUs, and 595.40 RFUs when the DNA splints harboring 1, 2 and 3 repeats of the binding sequence were used, respectively (FIG. 12B). Interestingly, the RNA splint also enhanced the reaction kinetics (FIG. 12B).

The transgenic cell line CHO-GFP-M20 has the transgene harboring 32 tandem copies of the probe-binding sequence M20³¹. To evaluate the BTT-DNA in enhancing the RNA template-driven (SEQ ID NO:14) proximity ligation in situ, cells were fixed and permeabilized, then reacted with CalFluor 647 azide via CuAAC in the presence of BTT-DNA ligand. Cells reacted with CalFluor 647 azide via CuAAC without an accelerating ligand were the negative control. Specific labeling of transcription sites in the nucleus and individual RNA molecules in the nucleus and cytoplasm was observed (FIG. 12C). The proximity ligation was applied to the parental CHO cell line that does not contain the transgene as a negative control.

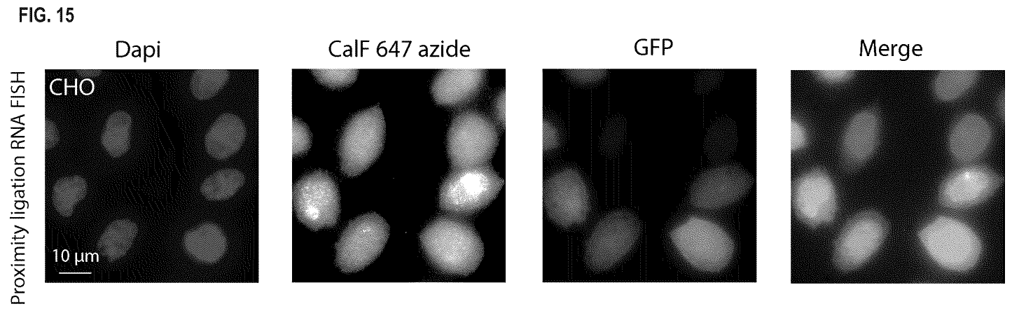

Equal concentrations of CHO cells and CHO-GFP-M20 cells were combined, cultivated, fixed, permeabilized, and then reacted with CalFluor 647 azide via CuAAC accelerated by BTT-DNA ligand. The cells with bright spots in the CalFluor 647 azide channel colocalize with GFP-positive cells, indicating that the probe was binding specifically (FIG. 15).

In Situ Proximity Ligation to Detect Individual mRNAs in Fixed Cells: No Wash Single Molecule Fluorescence In Situ Hybridization (smFISH).

Proximity ligation was applied to detect individual RNA molecules in the cell nucleus and cytoplasm using click RNA FISH assisted by the disclosed DNA oligomer-conjugated CuAAC ligand in fixed cells.

To test the proximity ligation in fixed cells, the transgenic cell line CHO-GFP-M20 was used, which contains a transgene including a GFP coding region and 32 tandem copies of the probe-binding sequence (M20) (FIG. 12D)³¹. The parental CHO cell line was used as a negative control, which does not contain the transgene. First, the CuAAC reaction time was optimized to bind an azido dye to the alkyne DNA probe in fixed CHO-GFP-M20 cells. The CuAAC reaction time was optimized and achieved the best labeling by performing a 1-hour CuAAC reaction (FIG. 12E).

Proximity click FISH of M20 transgene RNA was performed on fixed cells according to the following method. Cho cells and CHO-GFP-M20 cells were cultured in Dulbecco's modified Eagle's medium (DMEM) supplemented with 10% FBS for three days and harvested by centrifugation (300×g, 3 min) and resuspended in fresh media and seeded into the 8-well chamber (Fisher Scientific). An equal concentration of Cho cells and CHO-GFP-M20 cells were seeded together, and the total cell number was 60,000 cells/well, and the total volume per well was 300 μl and incubated for 24 hours. Cells were rinsed with 1×PBS and fixed with 4% formaldehyde for 10 minutes, then permeabilized with 70% EtOH in NF—H₂O at 4° C. overnight. Cells were rinsed with 1×PBS/10% formamide, followed by treatment with 100 μl single molecule fluorescence in situ hybridization (smFISH) hybridization buffer (10% formamide and 10% dextran sulfate in 1×PBS) containing 2 μl of M20-left-alkyne_20_2 (10 μM; SEQ ID NO: 6) for 24 hours at 37° C. After hybridization, cells were washed with 300 μl 1×PBS/10% formamide for 30 minutes at 37° C. Then, cells were treated with 10 μM CalFluor 647 azide, BTT-DNA ligand ([BTT-DNA]=20 μM), and 2.5 mM freshly prepared sodium ascorbate for 1 hour at 37° C. After the click reaction, the cells were re-washed with 300 μl 1×PBS/10% formamide for 30 minutes twice at 37° C. and counterstained with DAPI stain diluted with 1×PBS/10% formamide for 20 minutes at 37° C. (DAPI and Hoechst 33342 can be used interchangeably). Then, 1×PBS was added to each well to prepare for imaging. For the negative control, the cells were treated with M20-left-alkyne_20_2 without ligand.

After CuAAC reaction time optimization, the CuAAC reaction was performed using the BTT-DNA ligand in CHO-GFP-M20 cells with eight different 5′ alkyne DNA probes to find the best 5′ alkyne DNA probe for maximum ligation to the fluorogenic azide for transgene detection (FIG. 12F). Clean and robust labeling of transcription sites in the nucleus and individual RNA molecules in the nucleus and cytoplasm was observed via CuAAC in the presence of a 5′ alkyne DNA probe with a two-nucleotide distance from the BTT-DNA probe (FIG. 12D, FIG. 12F). Then, equal concentrations of CHO-GFP-M20 and parental CHO cells were seeded together. The cells were fixed and permeabilized, then reacted with CalFluor 647 azide via CuAAC in the presence of BTT-DNA ligand for 1 hour. Specific labeling of transcription sites in the nucleus and individual RNA molecules was observed in the nucleus and cytoplasm (FIG. 12D). The cells with bright puncta in the CalFluor 647 azide channel colocalized with GFP-positive cells, indicating that the probe was binding specifically (FIG. 12D).

### Example 6. DNA Detection

EdU Labeling of DNAs and Fixed Cell Detection with BTT-DNA Ligand.

The activity of BTT-DNA in driving the CuAAC reaction in a cellular environment to detect the various biomolecules in/on fixed cells was tested. EdU is an alkyne-derivatized thymidine analog that was metabolically incorporated into newly synthesized DNAs during active DNA synthesis in live cells. Following overnight metabolic incorporation, cells were fixed and permeabilized, then reacted with CalFluor 647 azide via CuAAC.

Specifically, HeLa cells were cultured in Dulbecco's Modified Eagle's Medium (DMEM) supplemented with 10% FBS for three days, harvested by centrifugation (300×g for 3 minutes), resuspended in fresh medium, and seeded into an 18-well chamber. A volume of 1.5 μl 5-ethynyl-2′-deoxyuridine (EdU) (1 mM, Thermo Fisher Scientific) was added to each well. The total volume per well was 150 μl. Cells were incubated for 24 hours. Cells were rinsed with 1×PBS, fixed with 4% formaldehyde for 10 minutes, then permeabilized with 70% EtOH in NF—H₂O at 4° C. overnight. Cells were washed with 1×PBS/10% formamide for 30 minutes at 37° C., followed by treatment with 10 μM CalFluor 647 azide, premixed BTTAA-CuSO₄ complex ([BTTAA]:[CuSO₄]=2:1, [CuSO₄]=10-30 μM) or BTT-DNA ligand ([BTT-DNA]=20 μM), and 2.5 mM freshly-prepared sodium ascorbate for 30 minutes at 37° C. After the click reaction, the cells were washed twice with 150 μl wash buffer for 20 minutes at 37° C. Cells were then counterstained with DAPI (4′,6-diamidino-2-phenylindole; CAS No. 28718-90-3) stain diluted with 1×PBS/10% formamide for 30 minutes at 37° C. Then 1×PBS was added to each well to prepare for imaging.

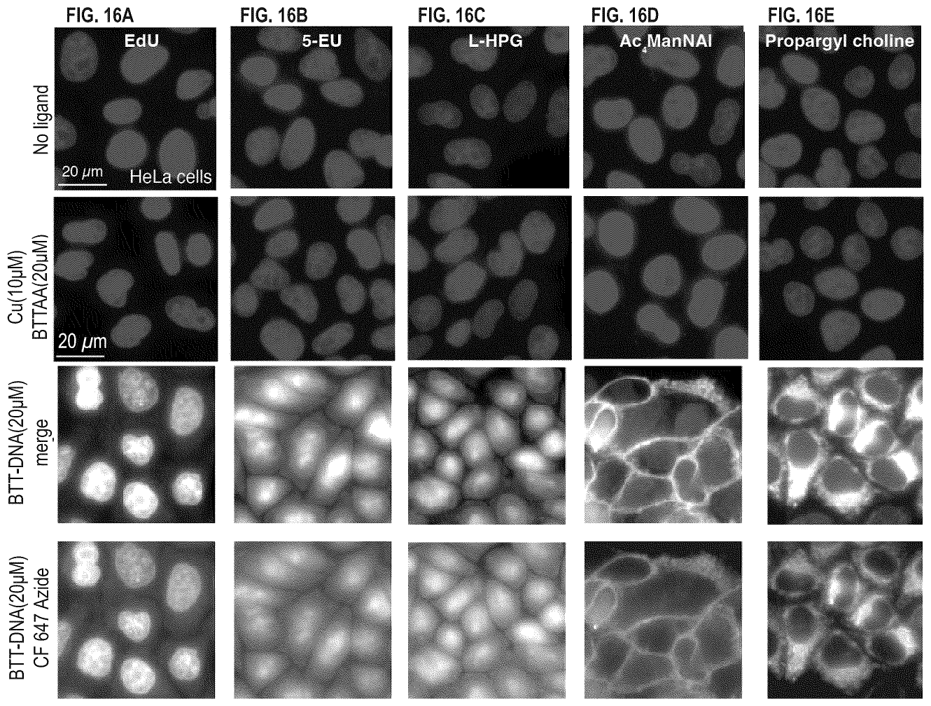

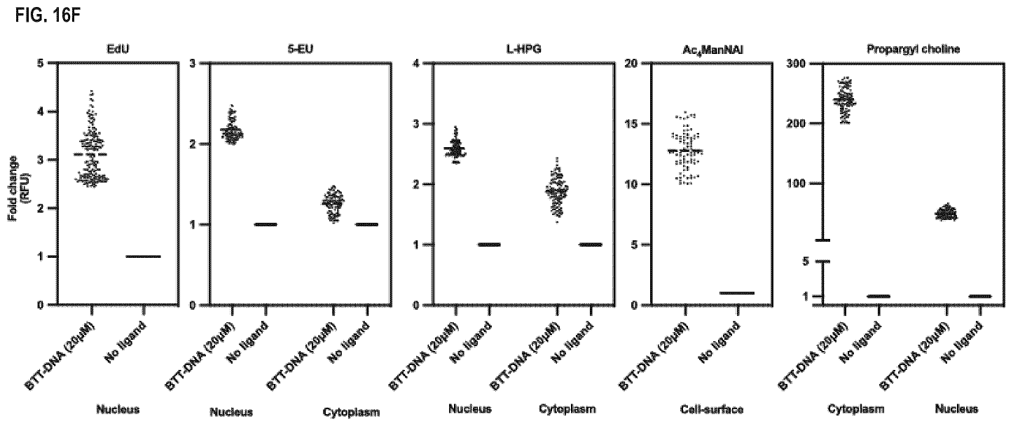

The performance of the BTT-DNA ligand was compared to commercially available BTTAA (FIG. 16A). The negative control containing no ligand showed very low background staining (1522.21 RFUs). Robust labeling of nascent DNA (20901.45 RFUs) was observed using the CuAAC accelerated by the BTT-DNA ligand (FIG. 16A), showing a 3-fold enhancement of signal over background at a ligand concentration of 20 M (FIG. 16A).

### Example 7. RNA Detection

EU Labeling of RNAs and Fixed Cell Detection with BTT_{(1,2)}-DNA Ligand.

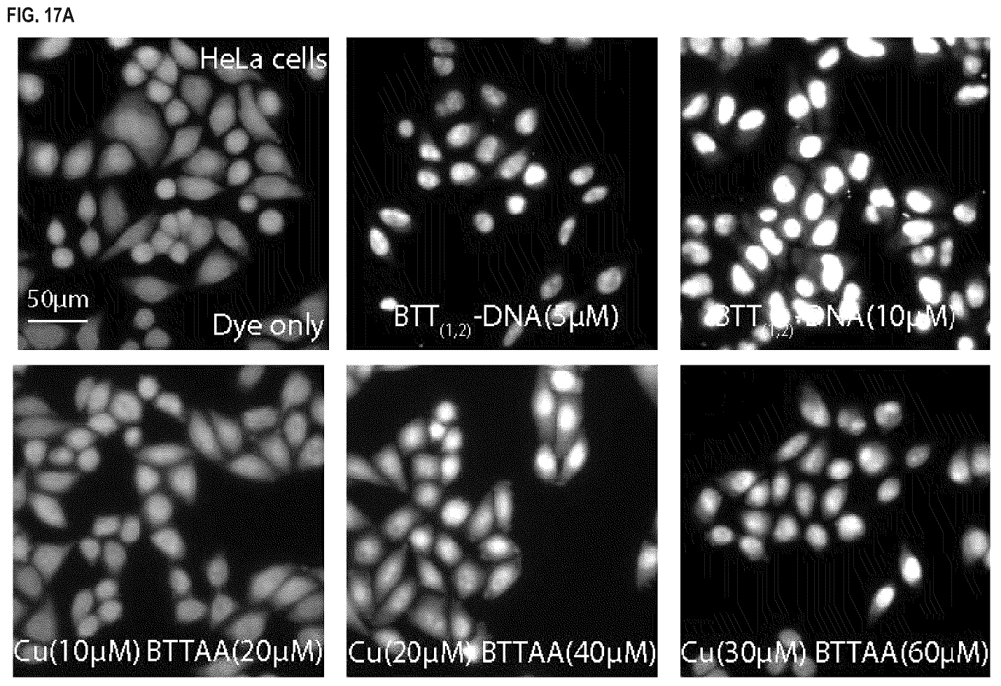

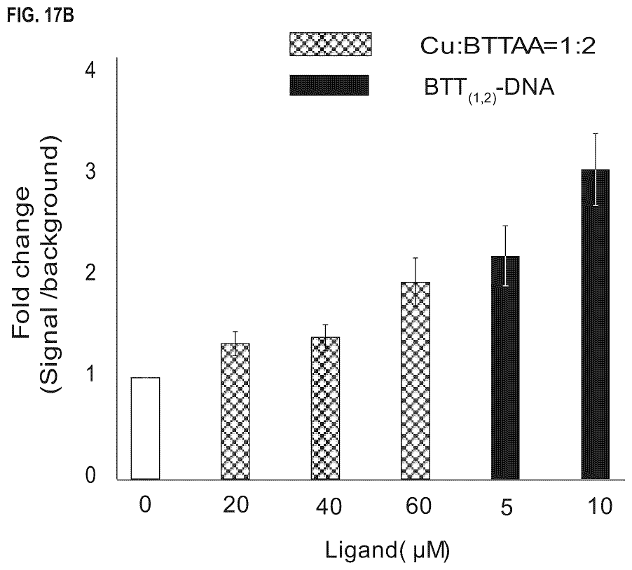

5-ethynyl uridine (EU) is an alkyne-derivatized uridine analog that was metabolically incorporated into nascent RNAs in live cells. Following metabolic incorporation, cells were fixed and permeabilized, then reacted with CalFluor 647 azide using CuAAC, comparing the performance of BTT_{(1,2)}-DNA ligand to commercially available BTTAA (FIGS. 17A-17B).

Specifically, HeLa cells were cultured in Dulbecco's Modified Eagle's Medium (DMEM) supplemented with 10% FBS for three days, harvested by centrifugation (300× g for 3 minutes), resuspended in fresh medium, and seeded into an 18-well chamber. The total volume per well was 150 μl. After one day of incubation at 37° C., 1.5 μl 5-ethynyl uridine (EU) was added to each well (1 mM final concentration) and returned to the incubator for a 3-hour incubation. Cells were rinsed with 1×PBS, fixed with 4% formaldehyde for 10 minutes, then permeabilized with 70% EtOH in NF—H₂O at 4° C. overnight. Cells were washed with 2× saline sodium citrated buffer (SSC)/10% formamide for 30 minutes at 37° C., followed by treatment with 10 μM CalFluor 647 azide, premixed BTTAA-CuSO₄ complex ([BTTAA]: [CuSO₄]=2:1, [CuSO₄]=0.5-30 μM) or BTT_{(1,2)}-DNA ligand ([BTT_{(1,2)}-DNA]=1-10 μM), and 2.5 mM freshly prepared sodium ascorbate for 30 minutes at 37° C. After click reaction, the cells were washed with 150 μl wash buffer for 30 minutes at 37° C. Cells were counterstained with DAPI stain diluted with 2×SSC/10% formamide for 20 minutes at 37° C. Then 2×SSC was added to each well to prepare for imaging.

The total fluorescent signal produced in the nucleus from EU labeling in the presence of the BTT_{(1,2)}-DNA ligand was 3.36-fold higher than the BTTAA-accelerated sample (FIGS. 17A-17B, FIG. 10C). The BTT_{(1,2)}-DNA enhanced CuAAC with a 12-fold lower ligand concentration (5 μM BTT_{(1,2)}-DNA compared to 60 M BTTAA; FIG. 10C). The BTT_{(1,2)}-DNA ligand produced a 3.1 signal-to-noise ratio while the commercial ligand produced a ratio of 1.94 (FIGS. 17A-17B, FIG. 10C).

EU Labeling of RNAs and Live Cell Detection with BTT_{(1,2)}-DNA Ligand.

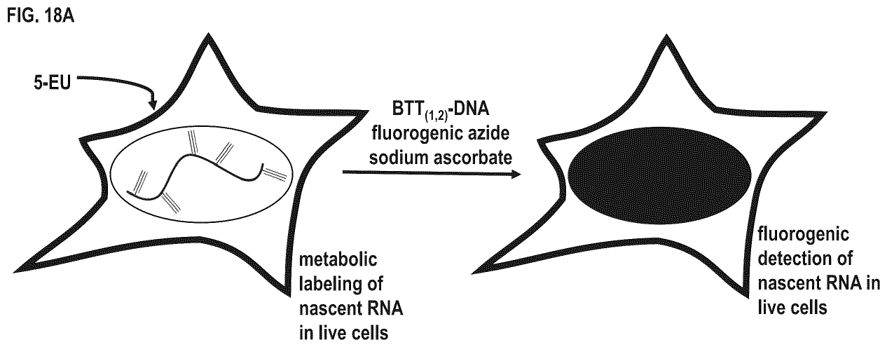

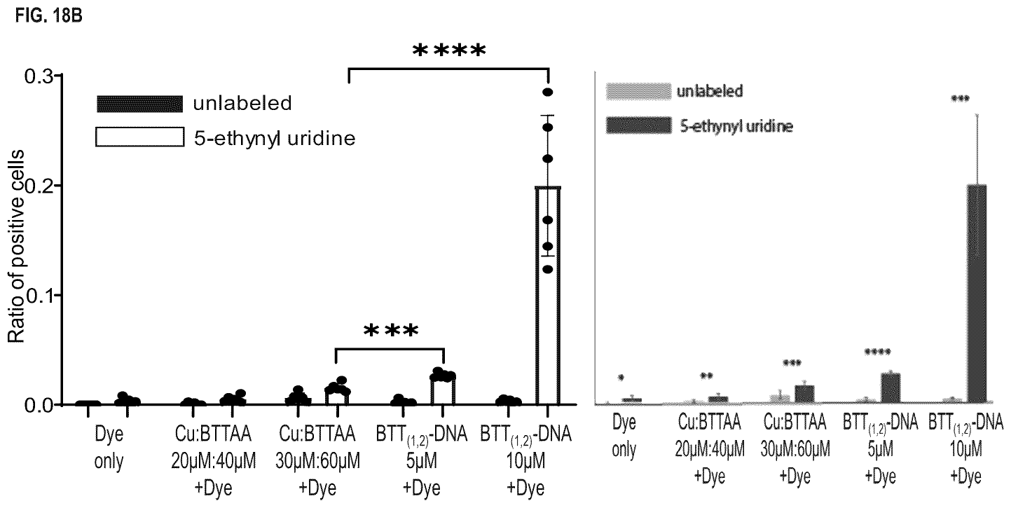

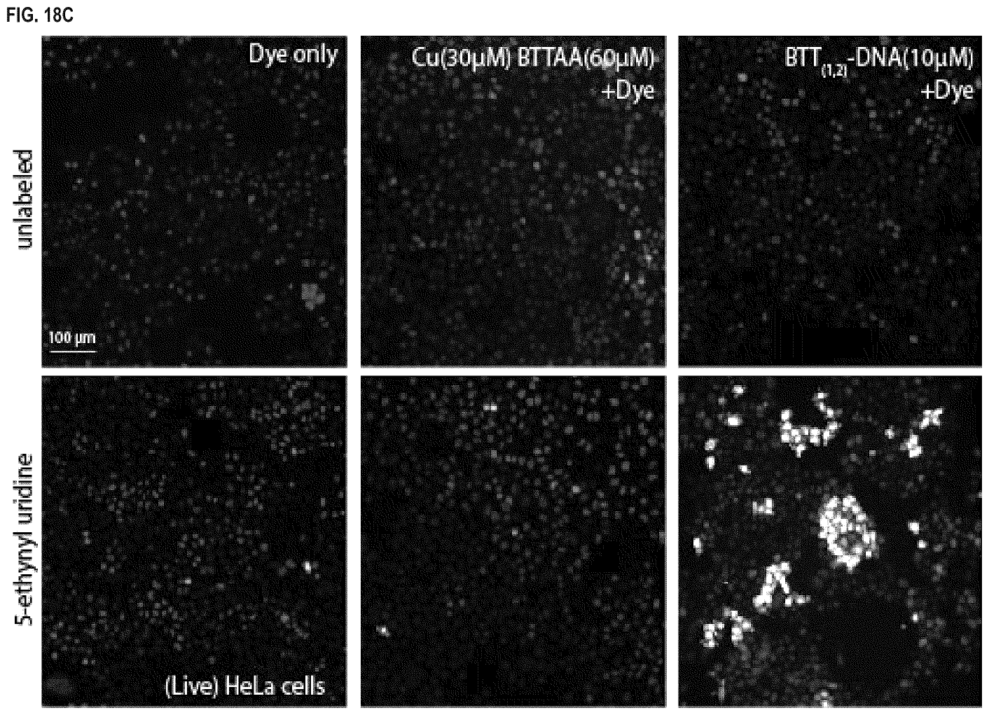

To evaluate the efficacy of the BTT_{(1,2)}-DNA ligand in driving CuAAC in the detection of intracellular biomolecules, nascent RNAs in Hela cells were metabolically labeled using 5-EU (FIG. 18A). Briefly, the HeLa cells were cultured with EU for 3 hours, then the CuAAC component was added directly to the live cells with a fluorogenic dye.

Specifically, HeLa cells were cultured in Dulbecco's Modified Eagle's Medium (DMEM) supplemented with 10% FBS for three days, harvested and resuspended in fresh medium, and seeded into an 18-well chamber. The total volume per well was 150 μl. After one day of incubation at 37° C., 1.5 μl 5-ethynyl uridine (EU) was added to each well (1 mM final concentration) and returned to the incubator for a 3-hour incubation. The negative control was the untreated cells. The cells were washed twice with PBS/1% FBS (pH 7.4) and treated with 100 μl of Invitrogen™ NUCBLUE® Live READYPROBES® reagent for 30 minutes at 37° C. to stain the nuclei of the live cells. Cells were washed three times with PBS/1% FBS (pH 7.4), followed by treatment with 10 μM CalFluor 647 azide (Click Chemistry Tools, Vector Laboratories, Newark, CA), premixed BTTAA-CuSO₄ complex ([BTTAA]: [CuSO₄]=2:1, [CuSO₄]=10-30 μM) or BTT_{(1,2)}-DNA ligand ([BTT_{(1,2)}-DNA]=5-10 μM), and 2.5 mM freshly prepared sodium ascorbate for 30 minutes in a 5% CO₂ incubator at 37° C. The cells were washed three times with 200 μl of PBS/1% FBS (pH 7.4) to remove the unreacted CalFluor 647 azide dye. Then 100 μl of PBS/1% FBS (pH 7.4) was added to each well to prepare for imaging.

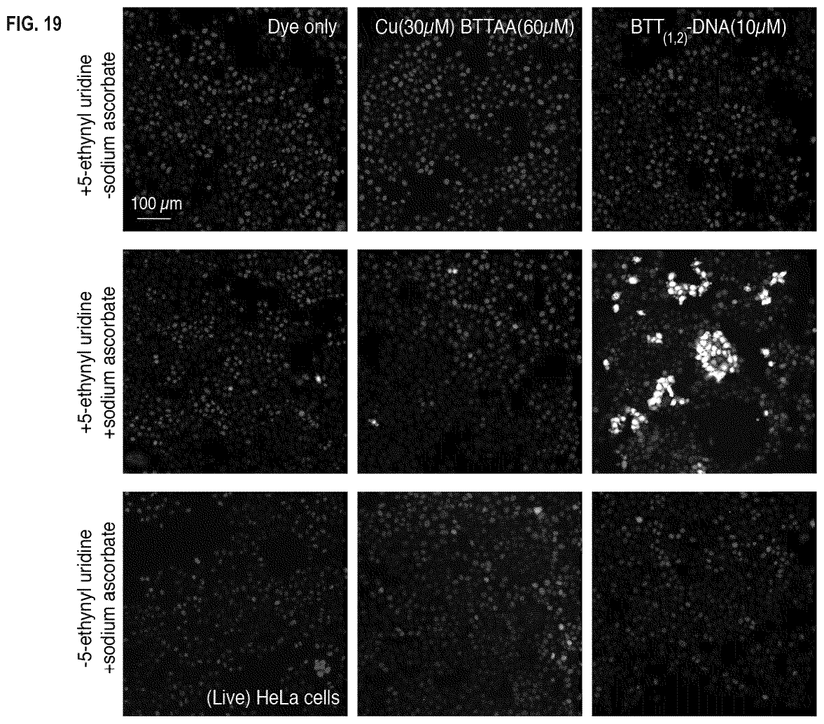

Using the BTT_{(1,2)}-DNA ligand to click the fluorogenic dye, positive signal was observed in 20% of the cell population (FIGS. 18B-18C, FIG. 19). When the BTT_{(1,2)}-DNA ligand concentration was lowered to 5 μM, a significant difference was still observed, however to a lesser extent.

EU Labeling of RNAs and Fixed Cell Detection with BTT-DNA Ligand.

5-ethynyl uridine (EU) is an alkyne-derivatized uridine analog that was metabolically incorporated into nascent RNAs in live cells (Jao and Salic 2008). Following a three-hour metabolic incorporation, cells were fixed and permeabilized, then reacted with CalFluor 647 azide via CuAAC. The performance of BTT-DNA ligand was compared to that of commercially available BTTAA (FIG. 16B).

HeLa cells were cultured in Dulbecco's Modified Eagle's Medium (DMEM) supplemented with 10% FBS for three days, harvested by centrifugation (300×g for 3 minutes), resuspended in fresh medium, and seeded into an 18-well chamber. The total volume per well was 150 μl. After one day of incubation at 37° C., 1.5 μl 5-ethynyl uridine (EU) was added to each well (1 mM final concentration) and returned to the incubator for a 3-hour incubation. Cells were rinsed with 1×PBS, fixed with 4% formaldehyde for 10 minutes, then permeabilized with 70% EtOH in NF—H₂O at 4° C. overnight. Cells were washed with 1×PBS/10% formamide for 30 minutes at 37° C., followed by treatment with 10 μM CalFluor 647 azide, premixed BTTAA-CuSO₄ complex ([BTTAA]: [CuSO₄]=2:1, [CuSO₄]=10-30 μM) or BTT-DNA ligand ([BTT-DNA]=20 μM), and 2.5 mM freshly prepared sodium ascorbate for 30 minutes at 37° C. After click reaction, the cells were washed twice with 150 μl wash buffer for 30 minutes at 37° C. Cells were counterstained with DAPI stain diluted with 1×PBS/10% formamide for 20 minutes at 37° C. Then 1×PBS was added to each well to prepare for imaging.

For the cells treated with 20 M BTT-DNA ligand, the intensity was very strong in the cell nuclei, especially in the nucleoli (8122.51 RFUs) where abundant ribosomal RNA are transcribed (Jao and Salic 2008) (FIG. 16B). The total fluorescent signal produced in the nucleoli in the presence of the BTT-DNA ligand was 2.46-fold higher than that in the non-nucleoli region (FIG. 16B).

Click Intracellular Labeling of Exogenously Delivered RNA Nucleotides in Live Cells with SLO

Hela cells were cultured in Dulbecco's modified Eagle's medium (DMEM) supplemented with 10% FBS for three days, then harvested and resuspended in fresh media. Cells were seeded into an 18-well chamber (Cellvis, Mountain View, CA) (2,000 cells per well) to a total volume of 150 μl per well and were incubated for 24 hours.

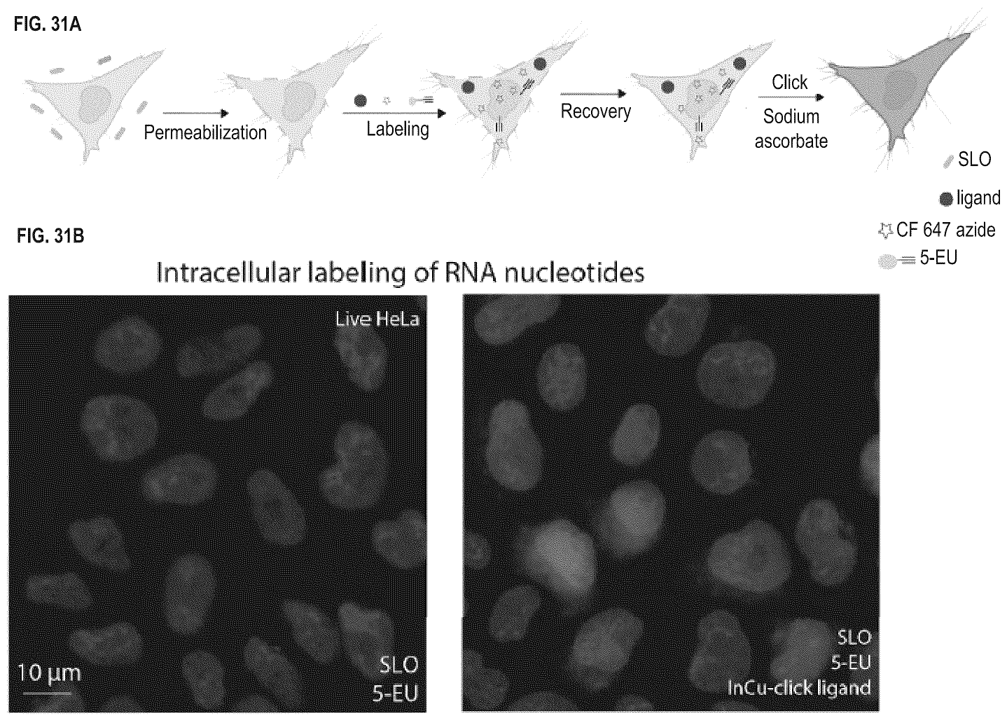

Cells were first treated with 200 ng/ml Streptolysin-O (SLO) (Bio-Rad, Hercules, CA) diluted with 1×HBSS (Thermo Scientific, Waltham, MA), which does not contain calcium and magnesium, for 12 minutes at 37° C. Cells were then washed with 3× with 1×HBSS without calcium and magnesium and treated with 200 μM CalFluor 647 azide and 1 mM 5-Ethynyl Uridine (5-EU) for 15 minutes. Cells were then washed three times with 1×HBSS without calcium and magnesium, followed by a 30-minute treatment of 30 μM BTT-DNA ligand diluted with 1×HBSS without calcium and magnesium. Cells were then washed 3× with non-phenol red DMEM/10% FBS to remove extra BTT-DNA ligand outside of the cells, followed by treatment with 2 mM freshly prepared sodium ascorbate for 20 minutes in a 5% CO₂ incubator at 37° C. The cells were washed 3× with 100 μl of non-phenol red DMEM/10% FBS to remove extra sodium ascorbate, followed by staining with 100 μl of diluted READYPROBES® Cell Viability Imaging Kit, Blue/Green (Thermo Fisher Scientific) for 5 minutes at 37° C. to stain the nucleus of the live and dead cells. The cells were then washed 3× with non-phenol red DMEM/10% FBS. Then 100 μl of non-phenol red DMEM/10% FBS was added to each well to prepare for imaging. For the negative control, untreated cells cultured in 5-EU and treated cells and treated cells cultured without 5-EU were included. See FIGS. 31A-31B.

Click Intracellular Labeling of Nascent RNAs in Live Cells with SLO

HeLa cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well) to a total volume of 150 μl per well, and incubated for 18 hours.

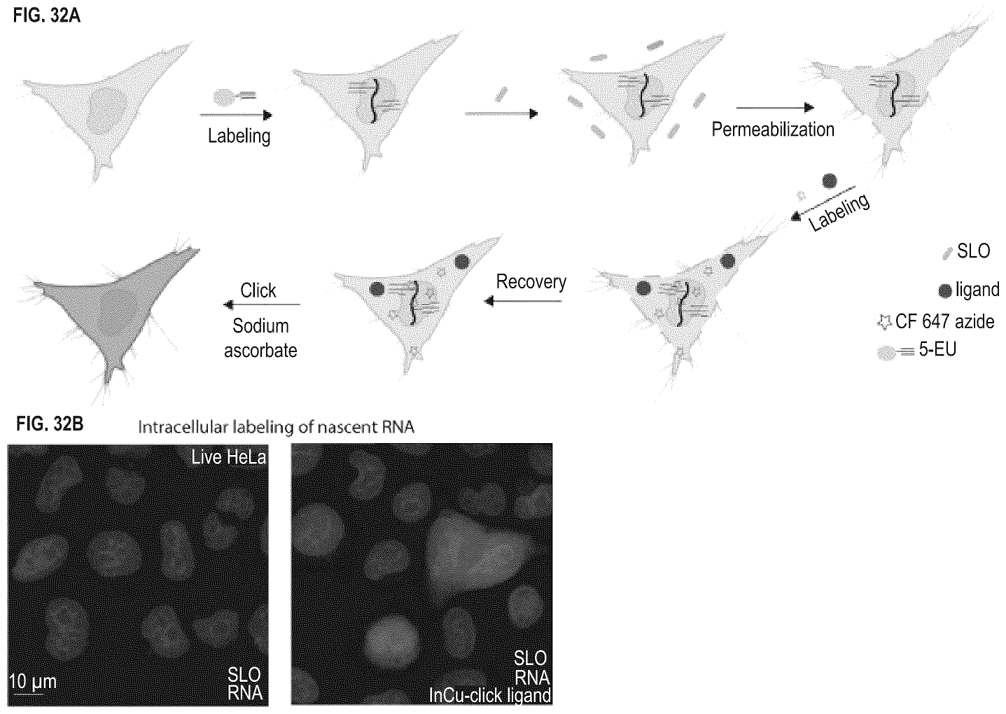

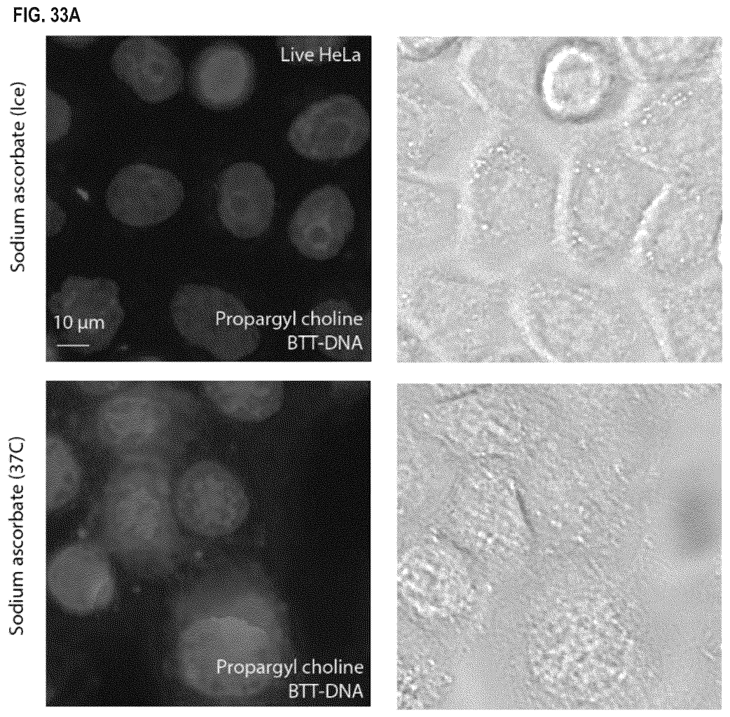

After an 18-hour incubation, cells were treated with 1 mM of 5-EU for 6 hours. Cells were then treated with 200 ng/ml Streptolysin-O (SLO) (Bio-Rad) diluted with 1×HBSS (Thermo Scientific), which does not contain calcium and magnesium, for 12 minutes at 37° C. Cells were then washed with 3× with 1×HBSS without calcium and magnesium and treated with 200 μM CalFluor 647 azide for 15 minutes and washed three times with 1×HBSS without calcium and magnesium, followed by a 30-minute treatment of 30 μM BTT-DNA ligand diluted with 1×HBSS without calcium and magnesium. Cells were then washed 3× with non-phenol red DMEM/10% FBS to remove extra BTT-DNA ligand outside of the cells, followed by treatment with 2 mM freshly prepared sodium ascorbate for 20 minutes in a 5% CO₂ incubator at 37° C. The cells were washed 3× with 100 μl of non-phenol red DMEM/10% FBS to remove extra sodium ascorbate, followed by staining with 100 μl of diluted READYPROBES® Cell Viability Imaging Kit, Blue/Green (Thermo Fisher Scientific) for 5 minutes at 37° C. to stain the nucleus of the live and dead cells. The cells were then washed 3× with non-phenol red DMEM/10% FBS. Then 100 μl of non-phenol red DMEM/10% FBS was added to each well to prepare for imaging. For the negative control, untreated cells cultured in 5-EU and treated cells and treated cells cultured without 5-EU were included. Sec FIGS. 32A-B.

### Example 8. Copper-Induced Cellular Toxicity

### Characterizing Copper-Induced Cellular Toxicity in the Presence of BTT_{(1,2)}-DNA Ligand

To evaluate the cytotoxicity of copper with the new Cu(I) accelerating ligand BTT_{(1,2)}-DNA, a) Jurkat cells that were treated with premixed BTTAA-CuSO₄ complex at the recommended copper concentration (20-30 μM) and sodium ascorbate were compared to b) Jurkat cells treated with BTT_{(1,2)}-DNA ligand in the optimal concentration range (5-10 μM; each molecule of ligand was complexed to approximately 7.5 molecules of copper; FIG. 1D) and sodium ascorbate. These samples were compared to untreated cells and cells reacted with 20-30 μM CuSO₄ and sodium ascorbate in the absence of ligand.

For the cell growth assay, Jurkat cells were grown in RPMI 1640 medium supplemented with 10% FBS. After 3 days, the cells were harvested by centrifugation (300×g for 3 minutes) and seeded into 48-well plates at a concentration of 1 million viable cells per well. The total volume of each well was 500 μl. Cells were treated with premixed BTTAA-CuSO₄ complex ([BTTAA]: [CuSO₄]=2:1, [CuSO₄]=20-30 μM) or BTT_{(1,2)}-DNA ligand ([BTT_{(1,2)}-DNA]=5-10 μM) and 2.5 mM freshly prepared sodium ascorbate. Untreated cells were used as a negative control; cells reacted with 20-30 μM CuSO₄ and 2.5 mM sodium ascorbate in the absence of BTTAA were used as the positive apoptosis control. Cells were incubated at 37° C. and viable cells were counted at 2, 6, 8, 10, and 12 hours using the Trypan blue dye exclusion method.

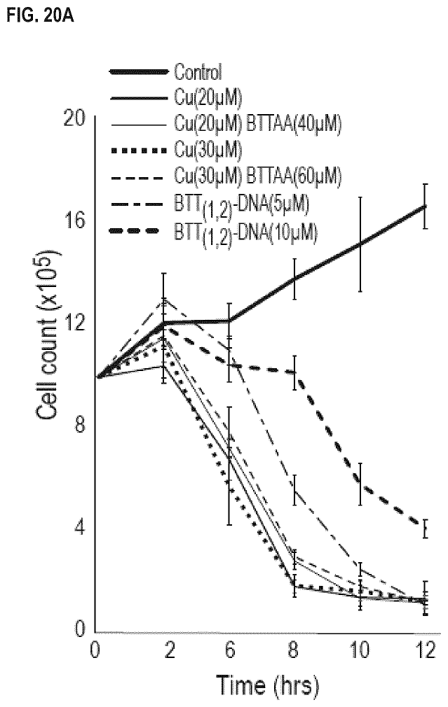

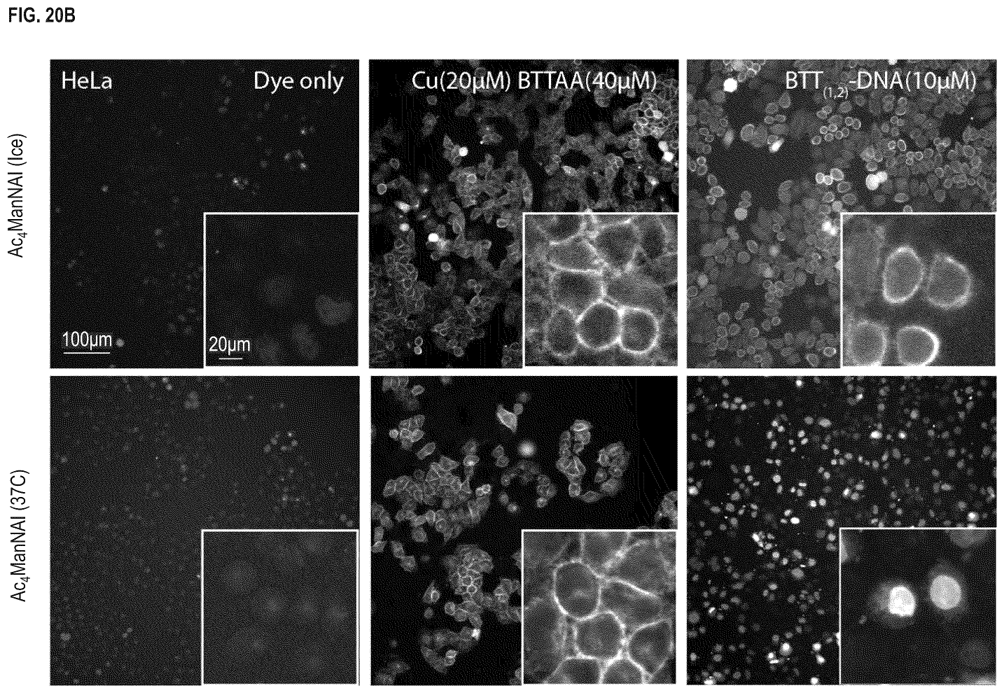

The new accelerating ligand, BTT_{(1,2)}-DNA ligand, outperformed the commercial BTTAA ligand for at least 12 hours at 10 μM (FIG. 20A). Interestingly, when the concentration was lowered to 5 μM, BTT_{(1,2)}-DNA ligand still outperformed the commercial BTTAA ligand, but not as significantly as BTT_{(1,2)}-DNA ligand at 10 UM after a 6-hour incubation (FIG. 20A).

### Characterizing Copper-Induced Cellular Toxicity in the Presence of BTT-DNA Ligand

### Methods

### Viability

HeLa cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well) to a total volume of 150 μl per well, and incubated for 24 hours.

12 μl of BTT-DNA ligand (100 μM) and 12 μl of M20-right_3′azide-complementary oligonucleotide (100 μM; SEQ ID NO:15) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and the tube was vortexed for 1 minute to dissolve the BTT-DNA and splint. After vortexing, tubes were mixed and hybridized for 1 hour at 37° C. After hybridization, the BTT-DNA ligand was diluted by adding 16.35 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 16.35 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

After encapsulation, 0.8 μl of sodium ascorbate (100 mM) was mixed with the DNA-lipid complex. Then, the mixture was applied to cells and incubated for 5 hours in a 5% CO₂ incubator at 37° C. Cells were then washed 3× with non-phenol red DMEM/10% FBS to remove extra BTT-DNA ligands outside of the cells, followed by staining with 100 μl of diluted READYPROBES® Cell Viability Imaging Kit, Blue/Green (Thermo Fisher Scientific) for 10 minutes at 37° C. to stain the nucleus of the live and dead cells. The cells were then washed 3× with non-phenol red DMEM/10% FBS. Then 100 μl of non-phenol red DMEM/10% FBS was added to each well to prepare for imaging. Untreated cells and cells treated with 2 mM sodium ascorbate were included for negative control. Cells were treated with 300 μM CuSO₄ in 2 mM sodium ascorbate for positive toxicity control.

The analysis of cell viability was conducted using CellProfiler³². Briefly, the DAPI and GFP channels of each image containing many cells were used for analysis. The DAPI channel was used to segment the nuclei by global minimum cross-entropy thresholding on the logarithm of intensity. Each nucleus was counted as one cell. The cells were classified as GFP-positive or GFP-negative based on the mean intensity of the GFP channel in the nucleus, where the threshold was set based on the intensity of the GFP channel from the untreated cells. The intensity of GFP-positive cells was higher than the threshold, and the intensity of GFP-negative cells was lower than the threshold. For the viability analysis, GFP-negative cells were viable cells with intact cell membranes, so they were accepted for further analysis. Sixteen fluorescence microscopy images from two biological experiments were obtained for the ratio of viable cells.

### ROS Detection

HeLa cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well) to a total volume of 150 μl per well and were incubated for 24 hours.

12 μl of BTT-DNA ligand (100 μM) and 12 μl of M20-right_3′azide-complementary oligonucleotide (100 μM) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and the tube was vortexed for 1 minute to dissolve the BTT-DNA and splint. After vortexing, the tubes were mixed and hybridized for 1 hour at 37° C. After hybridization, the BTT-DNA ligand was diluted by adding 16.35 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 16.35 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

After the encapsulation, 0.8 μl of sodium ascorbate (100 mM) was mixed to the DNA-lipid complex. Then, the mixture was applied to cells and incubated for 5 hours in a 5% CO₂ incubator at 37° C. Cells were then washed 3× with PBS/1% FBS (pH 7.4) to remove extra BTT-DNA ligands outside of the cells, followed by staining with 100 μl of diluted CELLROX® Green Reagent (Thermo Fisher Scientific) and NUCBLUE® Live READYPROBES® Reagent (Thermo Fisher Scientific) for 20 minutes at 37° C. The cells were then washed 3× with PBS/1% FBS (pH 7.4). Then 100 μl of PBS/1% FBS (pH 7.4) was added to each well to prepare for imaging. Untreated cells and cells treated with 2 mM sodium ascorbate were included for negative control. Cells were treated with 300 μM CuSO₄ in 2 mM sodium ascorbate for a positive toxicity control.

The analysis of ROS in cells was conducted using CellProfiler³². Briefly, the DAPI and GFP channels of each image containing many cells were used for analysis. The DAPI channel was used to segment the nuclei by global minimum cross-entropy thresholding on the logarithm of intensity. Each nucleus was counted as one cell. The cells were classified as GFP-positive or GFP-negative based on the mean intensity of the GFP channel in the nucleus, where the threshold was set based on the intensity of the GFP channel from the untreated cells. The intensity of GFP-positive cells was higher than the threshold, and the intensity of GFP-negative cells was lower than the threshold. For the ROS analysis, GFP-positive cells were cells with ROS, so they were accepted for further analysis. Sixteen fluorescence microscopy images from two biological experiments were obtained for the ratio of cells with ROS.

### Mitochondrial Superoxide Detection

HeLa cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well) to a total volume of 150 μl per well and were incubated for 24 hours.

12 μl of BTT-DNA ligand (100 μM) and 12 μl of M20-right_3′azide-complementary oligonucleotide (100 μM) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and the tube was vortexed for 1 minute to dissolve the BTT-DNA and splint. After vortexing, tubes were mixed and hybridized for 1 hour at 37° C. After hybridization, the BTT-DNA ligand was diluted by adding 16.35 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 16.35 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

After the encapsulation, 0.8 μl of sodium ascorbate (100 mM) was mixed into the DNA-lipid complex. Then, the mixture was applied to cells and incubated for 5 hours in a 5% CO₂ incubator at 37° C. Cells were then washed 3× with PBS/1% FBS (pH 7.4) to remove extra BTT-DNA ligands outside of the cells, followed by staining with 100 μl of diluted MITOSOX® Green superoxide indicators and NUCBLUE® Live READYPROBES® Reagent (Thermo Fisher Scientific) for 30 minutes at 37° C. The cells were then washed 3× with PBS/1% FBS (pH 7.4). Then 100 μl of PBS/1% FBS (pH 7.4) was added to each well to prepare for imaging. Untreated cells and cells treated with 2 mM sodium ascorbate were included for negative control. Cells were treated with 300 μM CuSO₄ in 2 mM sodium ascorbate for positive toxicity control.

For the analysis of mitochondrial superoxide, the threshold for the intensity of mitochondria was based on the intensity of mitochondria from untreated cells because the MITOSOX® Green superoxide indicators measure the superoxide produced only by mitochondria. Based on the images, all cells treated with copper in the presence of sodium ascorbate showed high fluorescent intensity in the mitochondria compared to the negative controls. The cells treated by BTT-DNA ligand were negative, so the positive cells were analyzed manually rather than using CellProfiler.

### Apoptosis Detection

Hela cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well), with or without propargyl choline, toto a total volume of 150 μl per well and incubated for 24 hours.

12 μl of BTT-DNA ligand (100 μM) and 12 μl of M20-right_3′azide-complementary oligonucleotide (100 μM) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and the tubed were vortexed for 1 minute to dissolve the BTT-DNA and splint. After vortexing, tubes were mixed and hybridized for 1 hour at 37° C. After hybridization, the BTT-DNA ligand was diluted by adding 16.35 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 16.35 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

After the encapsulation, 0.8 μl of sodium ascorbate (100 mM) was mixed to the DNA-lipid complex. Then, the mixture was applied to cells and incubated for 5 hours in a 5% CO₂ incubator at 37° C. Cells were then washed 3× with PBS/1% FBS (pH 7.4) to remove extra BTT-DNA ligands outside of the cells, followed by staining with 100 μl of diluted CELLEVENT® Caspase-3/7 Red Detection Reagent (Thermo Fisher Scientific) and NUCBLUE® Live READYPROBES® Reagent (Thermo Fisher Scientific) for 45 minutes at 37° C. The cells were then washed 3× with PBS/1% FBS (pH 7.4). Then 100 μl of PBS/1% FBS (pH 7.4) was added to each well to prepare for imaging. Untreated cells and cells treated with 2 mM sodium ascorbate were included for negative control. Cells were treated with 300 μM CuSO₄ in 2 mM sodium ascorbate for positive toxicity control.

### Results

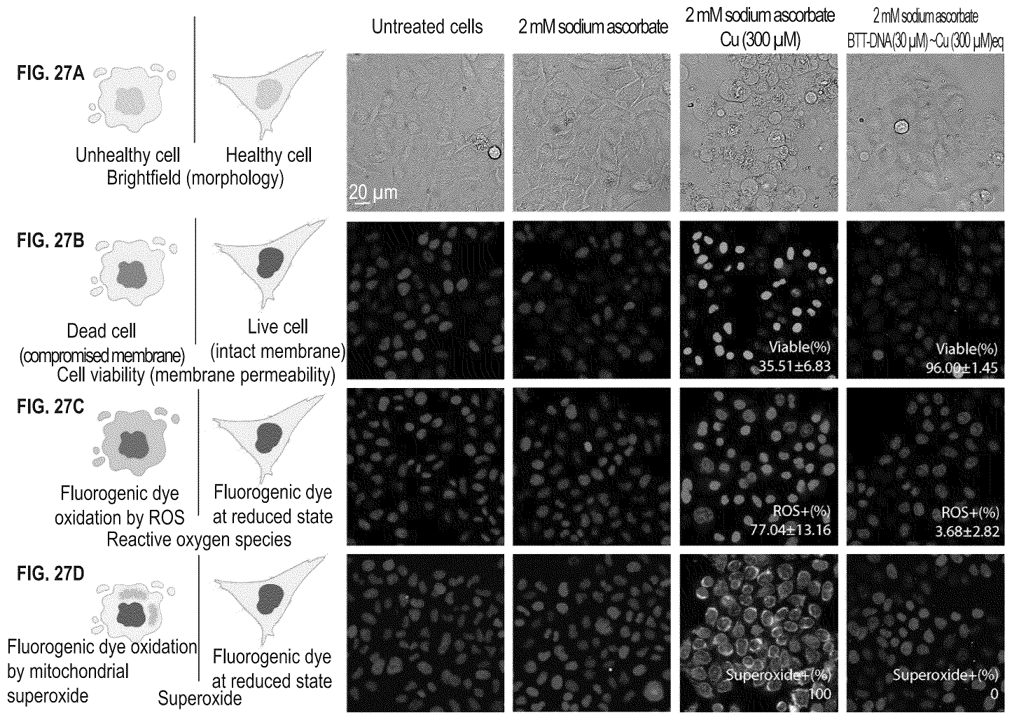

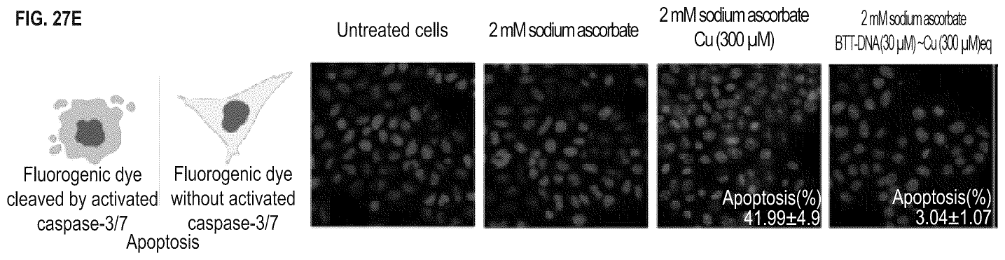

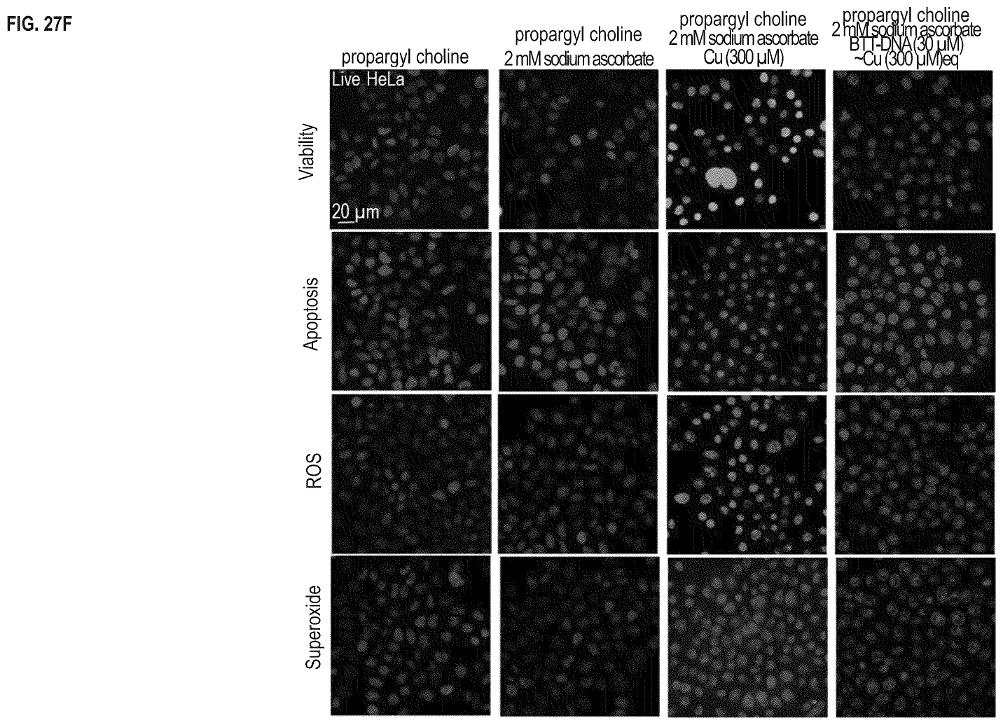

To evaluate the cytotoxicity of copper with the new Cu(I) accelerating ligand BTT-DNA, HeLa cells that were treated with CuSO₄ at the copper concentration (300 μM) were compared to HeLa cells treated with BTT-DNA ligand in the equivalent copper ion concentration (30 M; each molecule of ligand was complexed to approximately 10 copper ions; FIG. 2A) (FIG. 27F). Each of these was delivered under reducing conditions with sodium ascorbate, whose concentration (2 mM; FIG. 24A) and treatment time (5 hours; FIG. 24B) was optimized to eliminate the effect of sodium ascorbate on cells. The concentration of LIPOFECTAMINE® RNAIMAX® transfection reagents was also optimized to eliminate the fluence on cell biology (0.5 μl; FIG. 24C). Most cells treated with free CuSO₄ and sodium ascorbate were “blebbing” (e.g., cell membrane bulged out indicative of apoptosis). In contrast, the morphology of cells treated with just sodium ascorbate or BTT-DNA and sodium ascorbate together were comparable to the negative control (FIG. 27A). This suggests that BTT-DNA ligand can prevent the cell from “blebbing” induced by Cu(I).

Next, the viability of the cells was compared using the READYPROBES® cell viability imaging kit. The nuclei of cells with compromised membranes were stained with the SYTOX® green and measured with fluorescence microscopy. The viability ratio for cells treated with Cu(I) was calculated to be 35.51±6.83% viable. In contrast, the viability ratio for cells treated with BTT-DNA ligand was 96.00±1.45%. This strengthens the claim that the BTT-DNA ligand protects against Cu(I) induced toxicity (FIG. 27B).

The treated cells were also stained with CELLEVENT® Caspase-3/7 Red Detection Reagent, a fluorogenic probe which fluoresces when bound to DNA after being cleaved by activated caspase-3/7 in apoptotic cells (FIG. 27E). 41.99±4.9% of cells were apoptotic following treatment by free Cu(I), while the ratio for cells treated with BTT-DNA was 3.04±1.07% (FIG. 27E). These results strengthen the claim that the BTT-DNA ligand protects against Cu(I) induced toxicity.

A major concern for applying CuAAC into biological systems is the formation of reactive oxygen species (ROS), which can cause oxidative damage to proteins (Li et al., 2016). To compare ROS generated from the treatment of live cells with BTT-DNA ligand, the amount of the ROS in live cells was measured by CELLROX® Green Reagent, which fluoresces after oxidation by ROS. 77.04±13.16% of cells were positive for ROS staining after free Cu(I) treatment. In contrast, the ratio for cells treated with BTT-DNA was only 3.68±2.82% (FIG. 27C), indicating that BTT-DNA significantly reduces the ROS produced by Cu(I) delivery in live cells. The MITOSOX® green superoxide indicator, a fluorogenic dye that can readily oxidize by superoxide produced by mitochondria rather than ROS, was also used. For cells treated by Cu(I), all cells showed fluorescence in the cytoplasm, indicating superoxide production. All cells treated by BTT-DNA were negative for superoxide (FIG. 27D). Together, these results indicate that BTT-DNA has a protective effect from Cu(I) toxicity and prevents the formation of ROS and superoxide.

The BTT-DNA ligand was demonstrated to have significantly lowered the copper-induced cytotoxicity. When cells were treated with copper and sodium ascorbate for 5 hours, the morphology of cells was shown to have changed and almost all cells were “blebbing,” which, without being bound by any theory, might be because the treatment of high concentration of copper in the presence of sodium ascorbate caused apoptosis of the cells. Copper in the presence of sodium ascorbate was shown to be able to compromise the cell membrane integrity and cause cell death. Interestingly, compromised cell membranes were also observed with sodium ascorbate alone when the treatment time was increased to 6 hours. It was also found that not every blebbing or round cell showed fluorescence for SYTOX® green, and cells with normal morphology could also show fluorescence for SYTOX® green, which means blebbing was not the only contributor to the compromised cell membrane. Copper-treated cells also produced oxidative stress, which can damage the biological material in cells, like ROS, which can oxidize the CELLROX® green reagent. This reagent is a fluorogenic probe that is non-fluorescent in the reduced state and exhibits bright green photostable fluorescence after being oxidized by the ROS and bound to DNA. Extremely strong fluorescence was observed in the nucleus, while the fluorescence signal was dimmer in the cell cytoplasm, which might be because there was less DNA in the cytoplasm than in the nucleus. Strong fluorescence was also observed in the nucleus of cells treated with 2.5 mM sodium ascorbate, indicating that a high concentration of sodium ascorbate could also cause the production of ROS in live cells. Superoxide is also a kind of oxidative stress responsible for damaging biomolecule structure integrity. MITOSOX® green superoxide indicator measures the amount of the superoxide produced only by mitochondria. Strong fluorescence was seen in the cytoplasm for cells treated with copper in the presence of sodium ascorbate. The intensity was much higher in some spots close to the nucleus, which might be the mitochondria of the cells. The cells treated with BTT-DNA ligand looked like the negative control for all the tests. This indicates that the BTT-DNA ligand is biocompatible with living cells by lowering the cytotoxicity caused by copper, e.g., that the CuAAC may be performed within live cells while maintaining the health of the cell using the BTT-DNA ligand.

### Example 9. Protein Detection

L-HPG Labeling of Proteins and Fixed Cell Detection with BTT-DNA Ligand.

L-homopropargyl (L-HPG) is a cell-permeable alkyne probe that was metabolically incorporated into nascent proteins in methionine-starved cells (Shieh et al. 2015). Cells were treated for 2 hours with L-HPG, then fixed and permeabilized. These samples were then reacted with CalFluor 647 azide via CuAAC in the presence of BTT-DNA to visualize the newly synthesized proteins (FIG. 16C).

Specifically, HeLa cells were cultured in Dulbecco's Modified Eagle's Medium (DMEM) supplemented with 10% FBS for three days, harvested by centrifugation (300×g for 3 minutes), resuspended in fresh medium, and seeded into an 18-well chamber. The total volume per well was 150 μl. After one day of incubation at 37° C., the cell growth medium was removed and replaced with methionine-free DMEM medium, then cells were incubated for 30 minutes to deplete the methionine. Then 1.5 μl L-HPG was added to each well (2 mM final concentration) and returned to the incubator for a 2-hour incubation. Cells were rinsed with 1×PBS, fixed with 4% formaldehyde for 10 minutes, then permeabilized with 70% EtOH in NF—H₂O at 4° C. overnight. Cells were washed with 1×PBS/10% formamide for 30 minutes at 37° C., followed by treatment with 10 μM CalFluor 647 azide, premixed BTTAA-CuSO₄ complex ([BTTAA]: [CuSO₄]=2:1, [CuSO₄]=10-30 μM) or BTT-DNA ligand ([BTT-DNA]=20 μM), and 2.5 mM freshly prepared sodium ascorbate for 30 minutes at 37° C. After click reaction, the cells were washed twice with 150 μl wash buffer for 30 minutes at 37° C. Cells were then counterstained with DAPI stain diluted with 1×PBS/10% formamide for 20 minutes at 37° C. Then 1×PBS was added to each well to prepare for imaging.

Strong fluorescent signals were found in the nucleus (51647.84 RFUs) and the cytoplasm (20005.6 RFUs) using BTT-DNA (FIG. 16C).

Labeling of Intracellular Proteins and Live Cell Detection with BTT-DNA

### Methods

Hela cells were cultured in Dulbecco's modified Eagle's medium (DMEM) supplemented with 10% FBS for three days, then harvested and resuspended in fresh media. Cells were seeded into an 18-well chamber (Cellvis) (2,000 cells per well) to a total volume of 150 μl per well and incubated for 24 hours.

12 μl of BTT-DNA ligand (100 μM) and 12 μl of M20-right_3′azide-complementary oligonucleotide (100 μM) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and tubes were vortexed for 1 minute to dissolve the BTT-DNA and splint. After vortexing, tubes were mixed and hybridized for 1 hour at 37° C. After hybridization, the BTT-DNA ligand was diluted by adding 16.5 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 16.5 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

Cells were first treated with 200 ng/ml Streptolysin-O (SLO) (Bio-Rad) diluted with 1×HBSS (Thermo Scientific), which does not contain calcium and magnesium, for 10 minutes at 37° C. Cells were then washed with 3× with 1×HBSS without calcium and magnesium and treated with 200 μM CalFluor 647 azide for 15 minutes and washed three times with 1×HBSS without calcium and magnesium before liposome transfection.

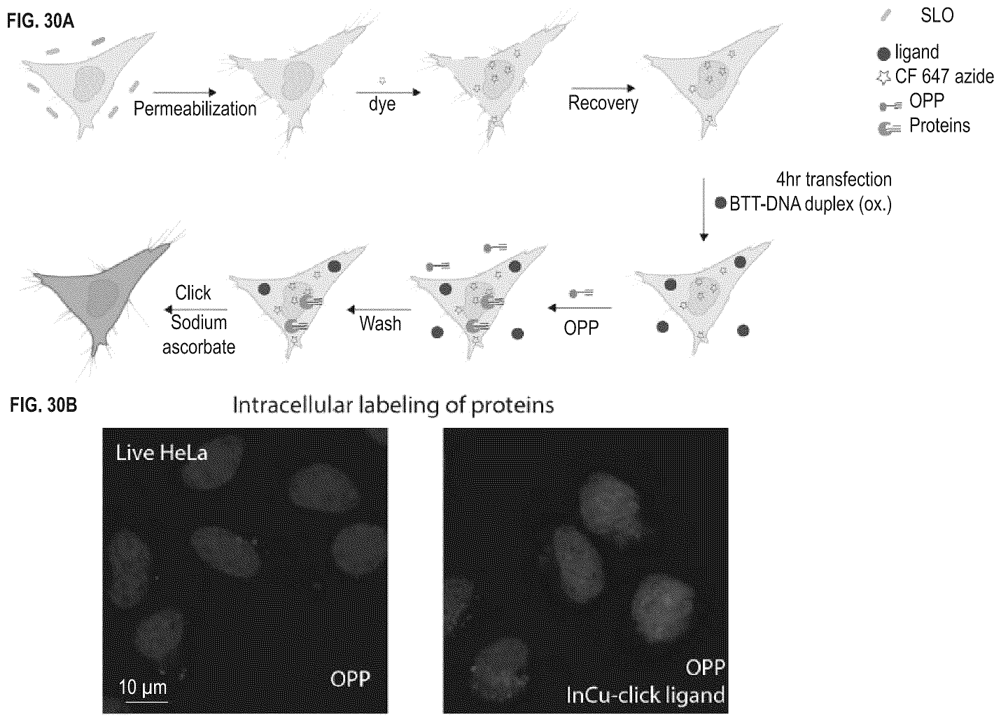

After the encapsulation, the DNA-lipid complex was applied to cells and incubated in a 5% CO₂ incubator at 37° C. After a 4-hour incubation, 0.4 μl of O-propargyl puromycin (OPP) (10 mM) was added to cells for one more hour of incubation. Cells were then washed 3× with non-phenol red DMEM/10% FBS to remove extra OPP and BTT-DNA ligand outside of the cells, followed by treatment with 2 mM freshly prepared sodium ascorbate for 25 minutes in a 5% CO₂ incubator at 37° C. The cells were washed 3× with 100 μl of non-phenol red DMEM/10% FBS to remove extra sodium ascorbate, followed by staining with 100 μl of diluted READYPROBES® Cell Viability Imaging Kit, Blue/Green (Thermo Fisher Scientific) for 5 minutes at 37° C. to stain the nucleus of the live and dead cells. The cells were then washed 3× with non-phenol red DMEM/10% FBS. Then 100 μl of non-phenol red DMEM/10% FBS was added to each well to prepare for imaging. For the negative control, untreated cells cultured in o-propargyl puromycin were included. See FIGS. 30A-B.

### Results

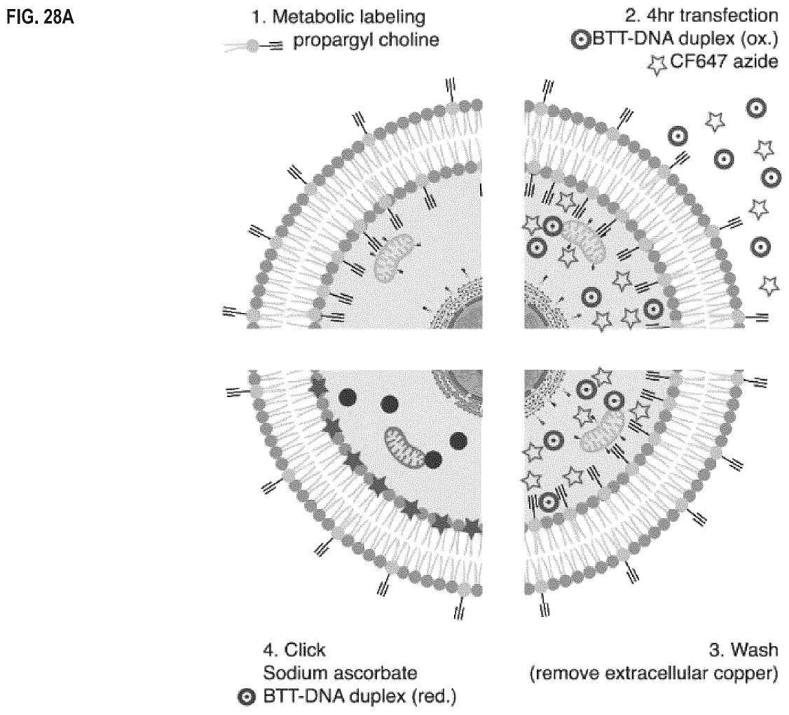

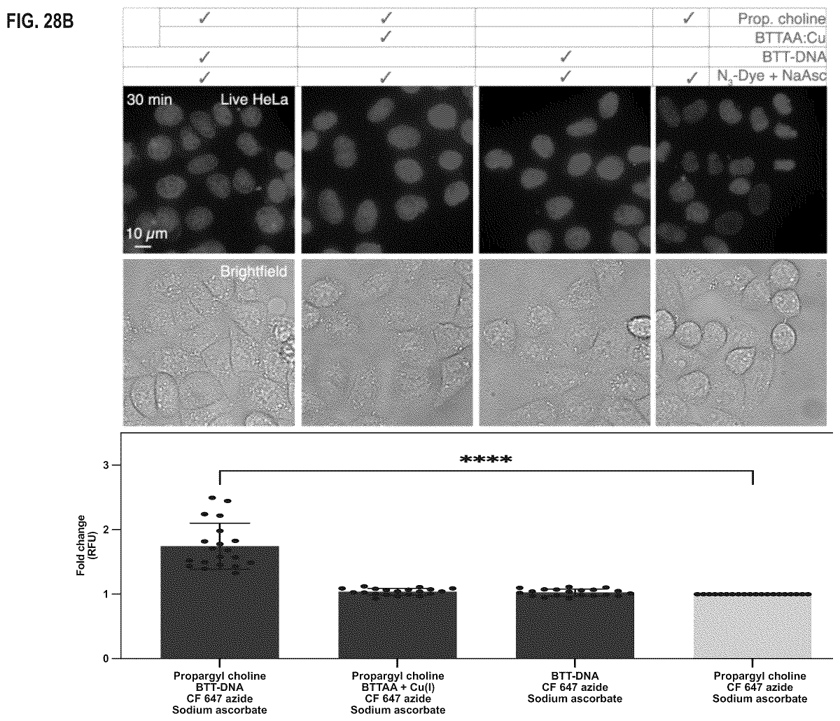

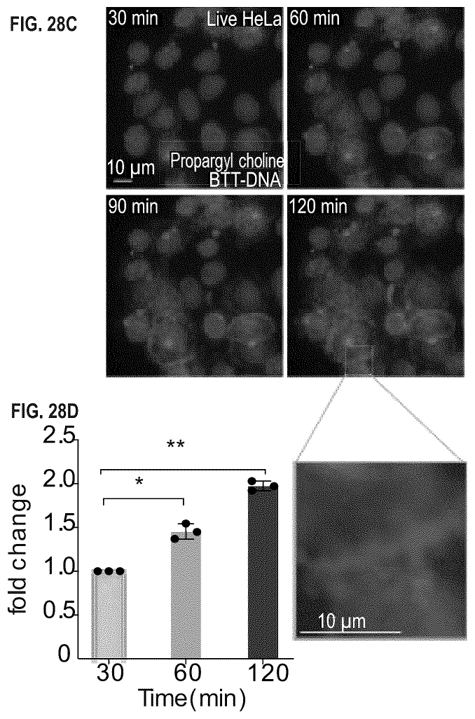

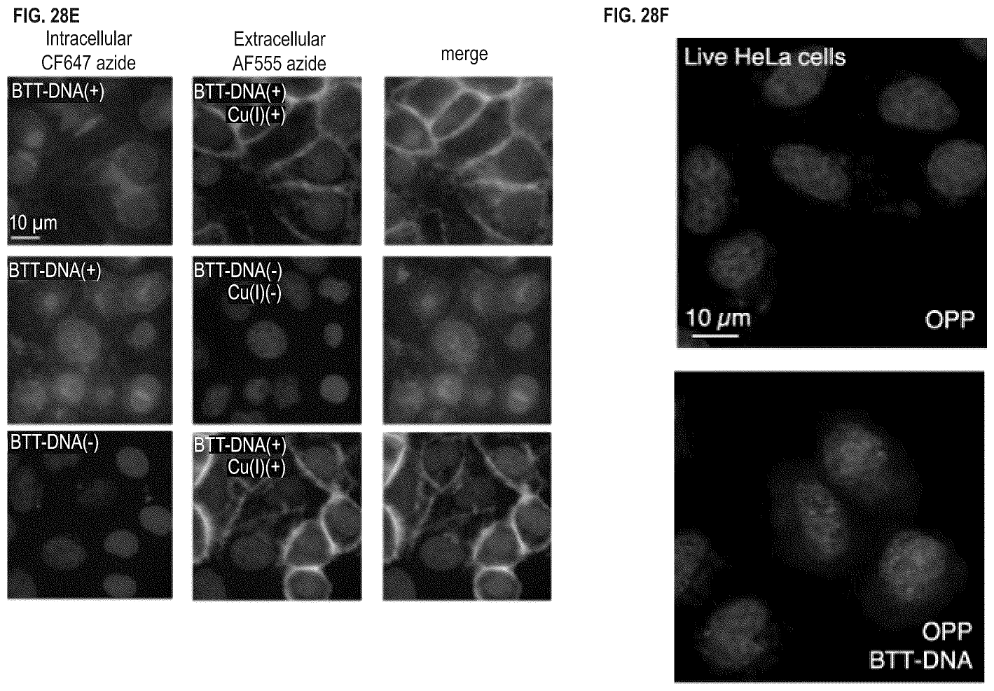

To expand the application of BTT-DNA to label and detect other major biomolecules, the intracellular labeling and detection of nascent proteins using O-propargyl puromycin (OPP), an alkyne analog of puromycin which can incorporate into newly synthesized proteins (Liu et al., 2012), was investigated. After 4 hours of transfection and 1 hour of OPP incubation, the cells were washed to ensure that CuAAC occurs exclusively inside the cells and not on extracellular alkynes, followed by a 30-minute sodium ascorbate incubation. Strong fluorescence intensity was observed in the cytoplasm and nucleus of cells treated with BTT-DNA (FIG. 28F), which was 1.38-fold higher than that of the no-ligand control (FIG. 28F). The result demonstrates that BTT-DNA ligand enables the detection of the exogenous delivered protein building blocks and intracellular proteins in live cells.

### Example 10. Cell Surface Sialic Acid Detection

Labeling of Cell-Surface Sialic Acids and Live Cell Detection with BTT_{(1,2)}-DNA Ligand.

To test the activity of BTT_{(1,2)}-DNA in driving the CuAAC reaction for live cells, HeLa cells were metabolically labeled with N-(4-pentynoyl)-mannosamine (Ac₄ManNAl; CAS No. 935658-93-8) to introduce an alkyne tag to cell surface sialic acids²⁵. A fluorophore was clicked to the alkynyl sugar, and cells were imaged using fluorescence microscopy.

Specifically, for click labeling of sialylated glycans on live cells, Hela cells were cultured in Dulbecco's Modified Eagle's Medium (DMEM) supplemented with 10% FBS for three days, then harvested and resuspended in fresh medium. A volume of 2.5 μl of Ac₄ManNAl (5 mM; Click Chemistry Tools) was added to each well of the 18-well chamber (Cellvis, Mountain View, CA) and dried for 20 minutes. Cells were seeded into the Ac₄ManNAl-treated 18-well chamber (total volume per well was 150 μl) and incubated for 24 hours. The cells were washed twice with PBS/1% FBS (pH 7.4) and treated with 100 μl of Invitrogen™ NUCBLUE® Live READYPROBES® reagent for 30 minutes at 37° C. to stain the nuclei of the live cells.

Cells were washed three times with PBS/1% FBS (pH 7.4), followed by treatment with 10 μM CalFluor 647 azide (Click Chemistry Tools), premixed BTTAA-CuSO₄ complex ([BTTAA]: [CuSO₄]=2:1, [CuSO₄]=0.5-30 μM) or BTT_{(1,2)}-DNA ligand ([BTT_{(1,2)}-DNA]=1-10 μM), and 2.5 mM freshly prepared sodium ascorbate for 30 minutes in a 5% CO2 incubator at 37° C. The cells were washed three times with 200 μl of PBS/1% FBS (pH 7.4) to remove the unreacted CalFluor 647 azide dye. Then 100 μl of PBS/1% FBS (pH 7.4) was added to each well to prepare for imaging.

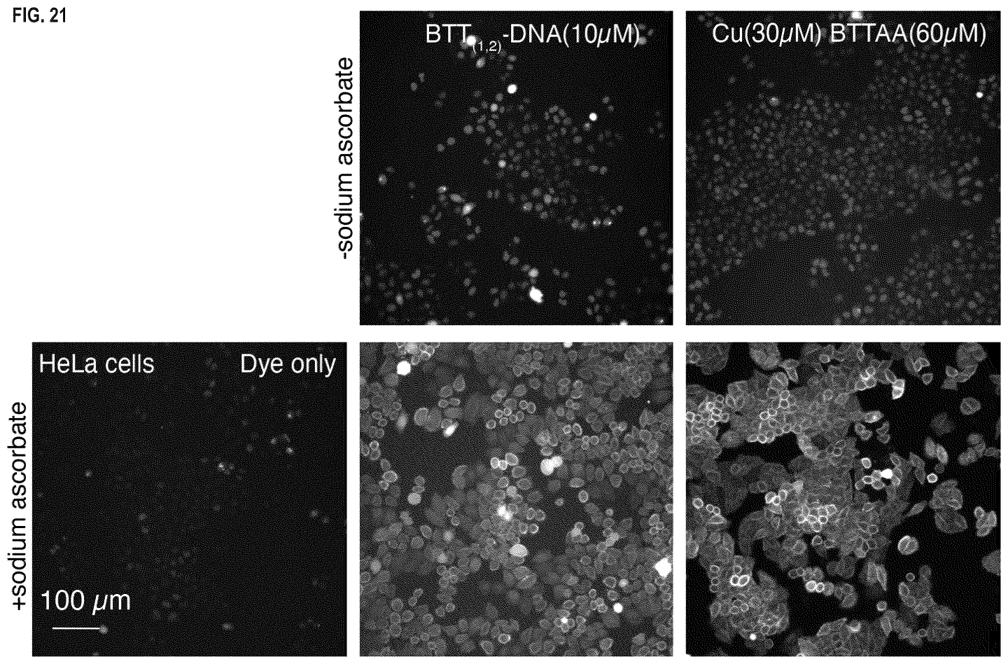

Following metabolic labeling, CuAAC was performed on ice (FIG. 20B, top row) as well as at 37° C. (FIG. 20B, bottom row). For the CuAAC on ice, the BTT_{(1,2)}-DNA-accelerated CuAAC produced equal fluorescent signal compared to the commercial ligand, but with a 4-fold lower ligand concentration (FIG. 20B, FIG. 21). At 37° C., the fluorescent signal on the membrane for the commercial ligand was equivalent to the signal observed on ice. Interestingly, for the CuAAC facilitated by the BTT_{(1,2)}-DNA ligand, many bright cells showed fluorescent signals localized in the nucleus, while the fluorescent signal on the membrane was 50% of the signal produced from the commercial ligand (FIG. 20B).

Labeling of Cell-Surface Sialic Acids and Fixed Cell Detection with BTT-DNA.

HeLa cells were metabolically labeled with Ac₄ManNAl to introduce an alkyne tag to cell surface sialic acids25, then a fluorophore was clicked to the alkynyl sugar and imaged using fluorescence microscopy (FIG. 16D).

For N-(4-pentynoyl)-mannosamine (Ac₄ManNAl) labeling of cell-surface sialic acid on fixed cells, HeLa cells were cultured in Dulbecco's Modified Eagle's Medium (DMEM) supplemented with 10% FBS for three days, then harvested and resuspended in fresh medium. A volume of 2.5 μl of Ac₄ManNAl (5 mM; Click Chemistry Tools) was added to each well of an 18-well chamber (Cellvis) and dried for 20 minutes. Cells were seeded into the Ac₄ManNAl-treated 18-well chamber (total volume per well was 150 μl) and incubated for 24 hours.

Cells were rinsed with 1×PBS and fixed with 4% formaldehyde for 10 minutes. Cells then were washed with 1×PBS/10% formamide for 30 minutes at 37° C., followed by treatment with 10 μM CalFluor 647 azide, premixed BTTAA-CuSO₄ complex ([BTTAA]:[CuSO₄]=2:1, [CuSO₄]=10-30 μM) or BTT-DNA ligand ([BTT-DNA]=20 μM), and 2.5 mM freshly prepared sodium ascorbate for 30 minutes at 37° C. After click reaction, the cells were washed twice with 150 μl wash buffer for 30 minutes at 37° C., and counterstained with DAPI stain diluted with 1×PBS/10% formamide for 20 minutes at 37° C. Then 1×PBS was added to each well to prepare for imaging.

Strong fluorescent signals were observed on the cell membrane (8531.51 RFUs) where the metabolically-labeled sialylated glycans were located (FIG. 16D).

Labeling of Cell-Surface Sialic Acids and Live Cell Detection with BTT-DNA.

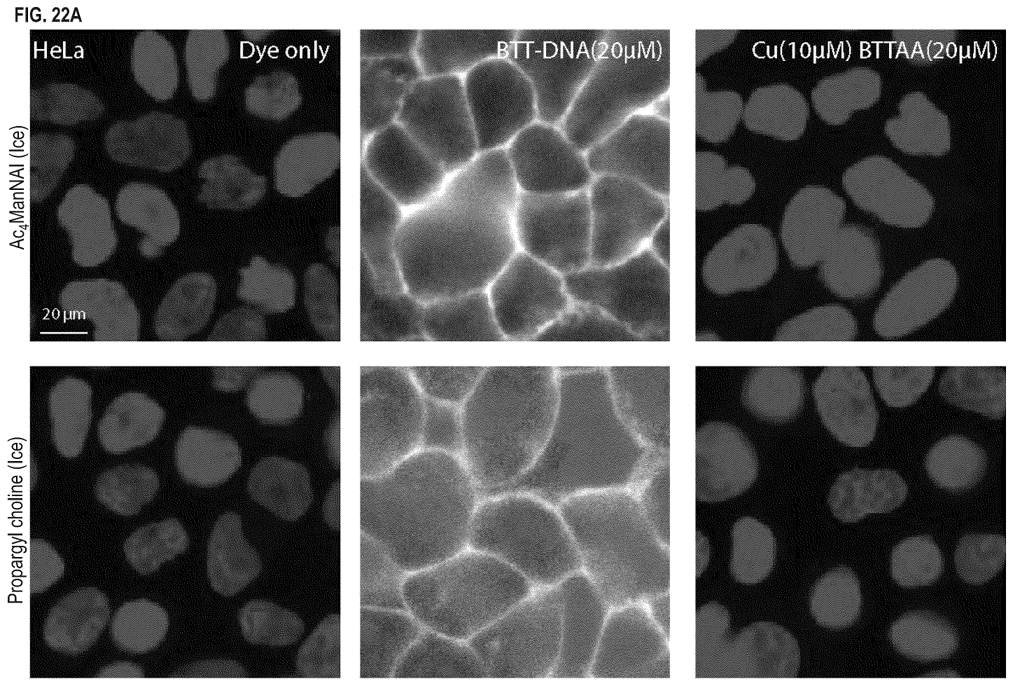

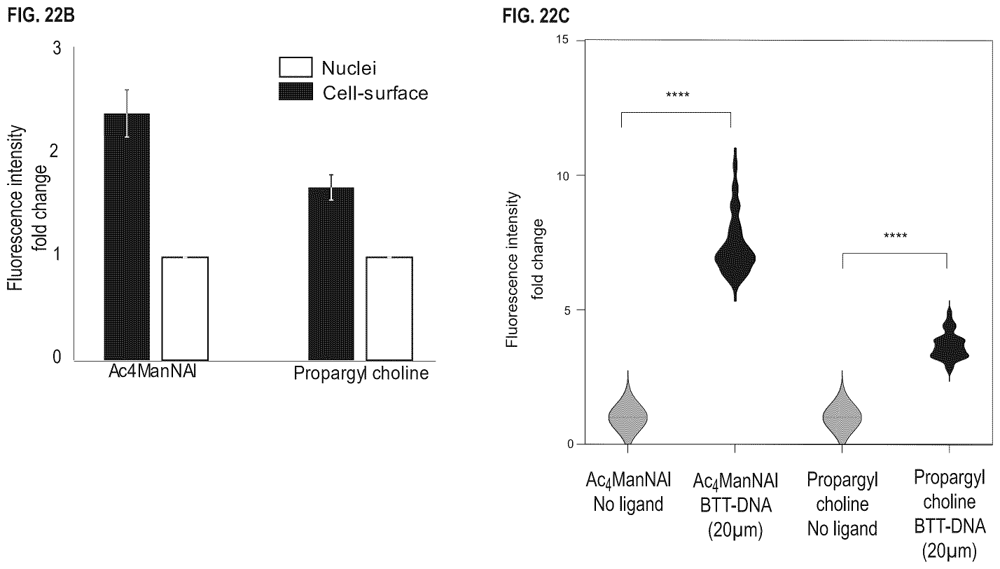

To test the activity of BTT-DNA in driving the CuAAC reaction for live cells, HeLa cells were metabolically labeled with Ac₄ManNAl (FIG. 22A), then CalFluor 647 azide was clicked to the alkynyl sugar on live cells.

For click labeling of sialylated glycans on live cells, HeLa cells were cultured in Dulbecco's Modified Eagle's Medium (DMEM) supplemented with 10% FBS for three days, then harvested and resuspended in fresh medium. A volume of 2.5 μl of Ac₄ManNAl (5 mM; Click Chemistry Tools) was added to each well of an 18-well chamber (Cellvis) and dried for 20 minutes. Cells were seeded into the Ac₄ManNAl-treated 18-well chamber (total volume per well was 150 μl) and incubated for 24 hours. The cells were washed twice with PBS/1% FBS (pH 7.4) and treated with 100 μl of Invitrogen™ NUCBLUE® Live READYPROBES® reagent for 30 minutes at 37° C. to stain the nuclei of the live cells.

Cells were washed three times with PBS/1% FBS (pH 7.4) followed by treatment with 10 μM CalFluor 647 azide (Click Chemistry Tools), premixed BTTAA-CuSO₄ complex ([BTTAA]:[CuSO₄]=2:1, [CuSO₄]=10-30 μM) or BTT-DNA ligand ([BTT-DNA]=20 M), and 2.5 mM freshly prepared sodium ascorbate for 30 minutes in a 5% CO₂ incubator at 37° C. The cells were washed three times with 200 μl of PBS/1% FBS (pH 7.4) to remove the unreacted CalFluor 647 azide dye. Then 100 μl of PBS/1% FBS (pH 7.4) was added to each well to prepare for imaging.

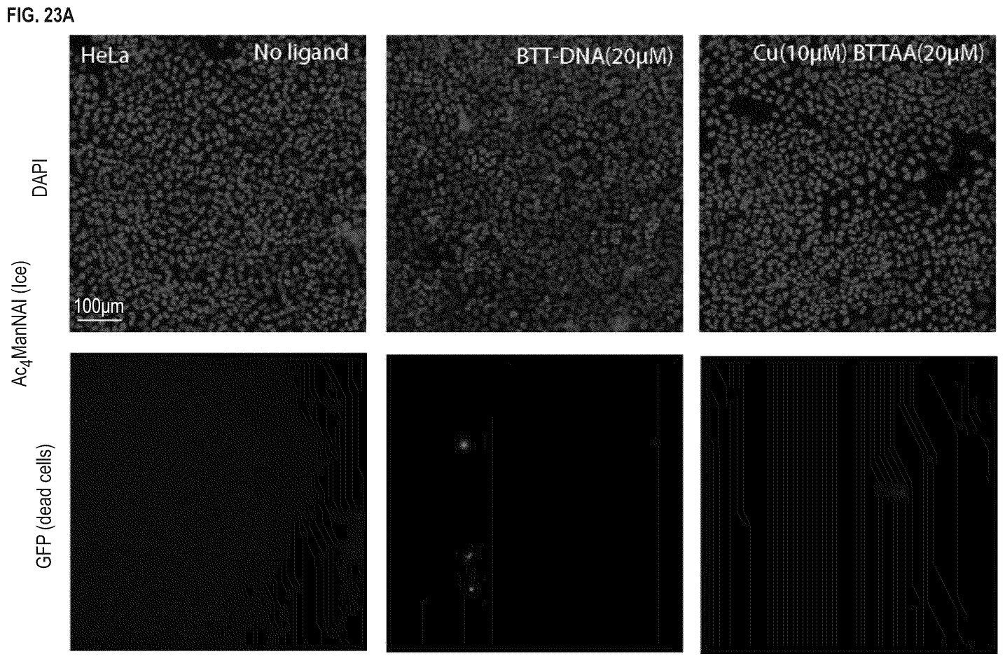

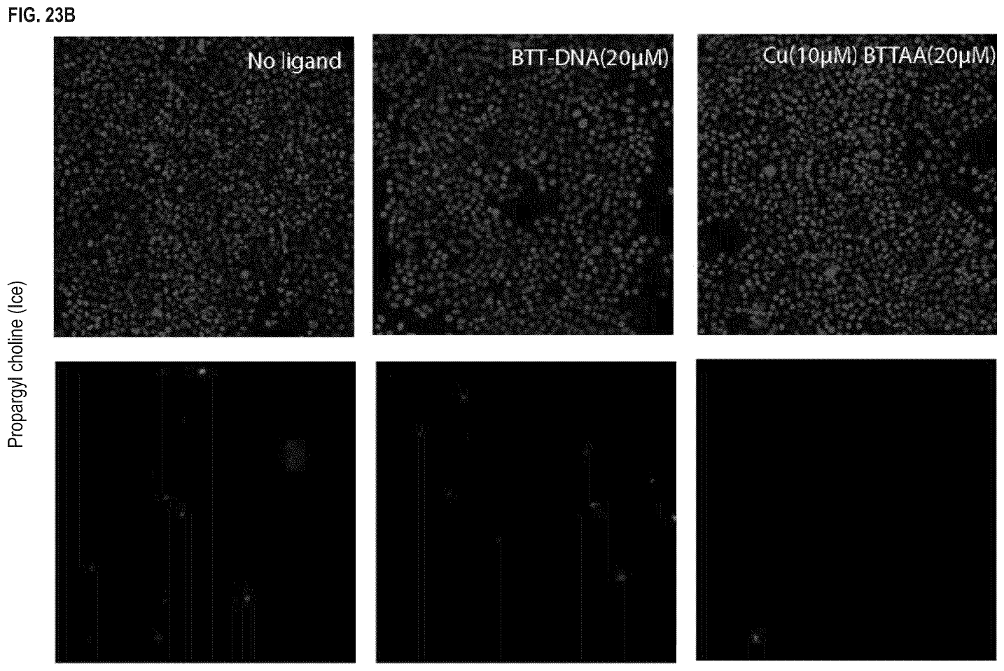

For the CuAAC on ice, the BTT-DNA accelerated CuAAC produced a robust fluorescent signal on the cell membrane. The signal over the background was 2.4-fold (FIG. 22B, FIG. 22C). Based on the live/dead staining result, cells were alive after 30 minutes of CuAAC reaction assisted by BTT-DNA ligand (FIG. 22A, FIG. 23A).

### Example 11. Phospholipid Detection

Labeling of Phospholipids and Fixed Cell Detection with BTT-DNA.

Propargyl choline (propargyl-Cho) was metabolically incorporated into newly synthesized phospholipids. Cells were incubated with propargyl-Cho for 24 hours, fixed and permeabilized, and then stained with CalFluor 647 azide via CuAAC in the presence of BTT-DNA to visualize the newly synthesized phospholipids (FIG. 16E).

For propargyl choline (propargyl-Cho) labeling of choline (Cho)-containing phospholipids on fixed cells, HeLa cells were cultured in Dulbecco's Modified Eagle's Medium (DMEM) supplemented with 10% FBS for three days, then harvested and resuspended in fresh medium. Cells were seeded into an 18-well chamber, 1.5 μl of propargyl choline (10 mM; Jena Bioscience) was added to each well (the total volume per well was 150 μl), and cells were incubated for 24 hours.

Cells were rinsed with 1×PBS and fixed with 4% formaldehyde for 10 minutes. Cells then were washed with 1×PBS/10% formamide for 30 minutes at 37° C., followed by treatment with 10 μM CalFluor 647 azide, premixed BTTAA-CuSO₄ complex ([BTTAA]:[CuSO₄]=2:1, [CuSO₄]=10-30 μM) or BTT-DNA ligand ([BTT-DNA]=20 μM), and 2.5 mM freshly prepared sodium ascorbate for 30 minutes at 37° C. After click reaction, the cells were washed twice with 150 μl wash buffer for 30 minutes at 37° C., and counterstained with DAPI stain diluted with 1×PBS/10% formamide for 20 minutes at 37° C. Then 1×PBS was added to each well to prepare for imaging.

The cells showed strong staining in the cellular membrane and the intracellular structure (46502.97 RFUs) (Jao et al. 2009) (FIG. 16E).

Labeling of Phospholipids and Live Cell Detection with BTT-DNA.

Cho-containing phospholipids were also detected by metabolic incorporation of the propargyl-Cho to evaluate the activity of BTT-DNA in driving the CuAAC reaction for extracellular labeling on live cells (FIGS. 22A, 23B).

For click labeling of Cho-containing phospholipids on live cells, HeLa cells were cultured in Dulbecco's Modified Eagle's Medium (DMEM) supplemented with 10% FBS for three days, then harvested and resuspended in fresh medium. Cells were seeded into an 18-well chamber, 1.5 μl of propargyl choline (10 mM; Jena Bioscience) was added to each well (the total volume per well was 150 μl), and cells were incubated for 24 hours. The cells were washed twice with PBS/1% FBS (pH 7.4) and treated with 100 μl of Invitrogen™ NUCBLUE® Live READYPROBES® reagent for 30 minutes at 37° C. to stain the nuclei of the live cells.

Cells were washed three times with PBS/1% FBS (pH 7.4), followed by treatment with 10 μM CalFluor 647 azide (Click Chemistry Tools), premixed BTTAA-CuSO₄ complex ([BTTAA]:[CuSO₄]=2:1, [CuSO₄]=10-30 μM) or BTT-DNA ligand ([BTT-DNA]=20 μM), and 2.5 mM freshly prepared sodium ascorbate for 30 minutes in a 5% CO₂ incubator at 37° C. The cells were washed three times with 200 μl of PBS/1% FBS (pH 7.4) to remove the unreacted CalFluor 647 azide dye. Then 100 μl of PBS/1% FBS (pH 7.4) was added to each well to prepare for imaging.

Robust labeling of the newly synthesized phospholipids in the cell membrane was found (FIGS. 22A-22C), demonstrating that the BTT-DNA ligand can drive CuAAC not only for extracellular and intracellular labeling on/in fixed cells, but also for extracellular labeling on live cells.

Labeling of Intracellular Phospholipids and Live Cell Detection with BTT-DNA.

The DNA oligo attachment of BTT-DNA serves several purposes, including facilitating intracellular labeling of nascent phospholipids in live cells. The following disclosure shows that BTT-DNA enabled the live-cell, intracellular labeling of nascent phospholipids.

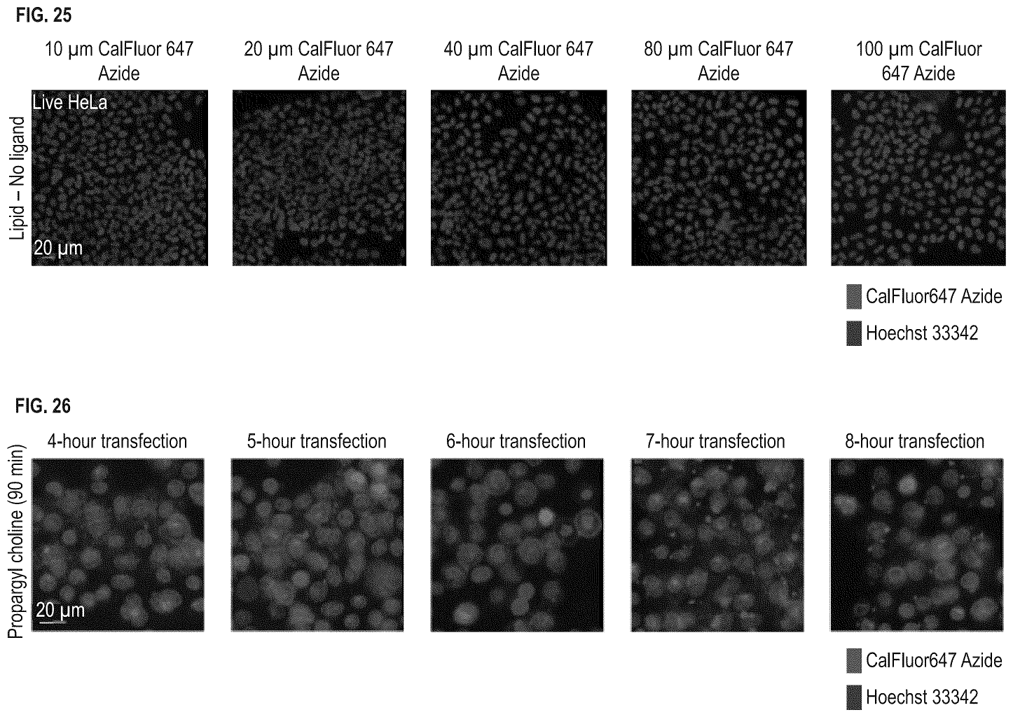

After a demonstration of the biocompatibility of the BTT-DNA ligand, intracellular biomolecule labeling was performed with CuAAC assisted by the BTT-DNA ligand. Metabolic incorporation of propargyl-choline into newly synthesized phospholipids by 24 hours of propargyl-choline treatment labeled the endoplasmic reticulum (ER), the Golgi, the mitochondrial and plasma membranes with choline-containing phospholipids. To ensure that the BTT-DNA ligand entered the cells to accelerate the CuAAC reaction, LIPOFECTAMINE® RNAIMAX® transfection reagent was used, which has high transfection efficiency for small interfering RNA (siRNA). Since LIPOFECTAMINE® RNAIMAX® encapsulates double-stranded nucleic acid, and BTT-DNA is single stranded, a short single-stranded DNA that is complementary to the sequence of BTT-DNA was designed and hybridized to form a duplex before transfection. After encapsulating the BTT-DNA duplex with the liposome, CalFluor 647 azide was added to the mixture at a concentration that leads to passive transport of dye into the cytoplasm while maintaining a sufficiently low dye background in live cells (FIG. 25). The transfection time was optimized to 4 hours to transport the duplex into the cytoplasm of cells (FIG. 26). After 4 hours of transfection, the cells were washed to ensure that CuAAC only happened inside of cells and not on extracellular alkynes, followed by a 30-minute sodium ascorbate incubation (FIG. 28A).

In detail, click intracellular labeling of choline-containing phospholipids in live cells with LIPOFECTAMINE® RNAIMAX® was performed as follows. Hela cells were cultured in Dulbecco's modified Eagle's medium (DMEM) supplemented with 10% FBS for three days, then harvested and resuspended in fresh media. Cells were seeded into an 18-well chamber (Cellvis) (2, 000 cells per well), 1.5 μl of propargyl choline (10 mM; Jena Bioscience) was added to each well to a total volume of 150 μl per well, and cells were incubated for 24 hours. 12 μl of BTT-DNA ligand (100 μM) and 12 μl of M20-right_3′azide-complementary oligonucleotide (100 μM) complementary to the BTT-DNA sequence were added to separate 1.5 mL EPPENDORF® DNA LOBIND® microcentrifuge tubes (Fisher Scientific) and dried out with SPEEDVAC® SPD1030 integrated vacuum concentrator (Thermo Scientific) at program 1 for 20 minutes. 3 μl of 1×PBS was added to each tube, and the tube was vortexed for 1 minute to dissolve the BTT-DNA and splint. After vortexing, tubes were mixed and hybridized for 1 hour at 37° C. After hybridization, the BTT-DNA ligand was diluted by adding 15.15 μl prewarmed OPTI-MEM® I reduced serum medium (Thermo Fisher Scientific) to the tube. At the same time, 0.5 μl of LIPOFECTAMINE® RNAIMAX® transfection reagent (Thermo Fisher Scientific) was diluted in 15.15 μl prewarmed OPTI-MEM® I reduced serum medium in a different 1.5 ml EPPENDORF® DNA LOBIND® microcentrifuge tube. Then, all the diluted BTT-DNA ligands were added to the LIPOFECTAMINE® RNAIMAX® transfection reagent and incubated at room temperature for 20 minutes to form the DNA-lipid complex.

After the encapsulation, 3.2 μl of CalFluor 647 azide (1 mM) was mixed into the DNA-lipid complex. Then, the mixture was applied to cells and incubated for 4 hours in a 5% CO₂ incubator at 37° C. Cells were then washed 3× with non-phenol red DMEM/10% FBS to remove extra dye and BTT-DNA ligand outside of the cells, followed by treatment with 2 mM freshly prepared sodium ascorbate for 30 minutes in a 5% CO₂ incubator at 37° C. The cells were washed 3× with 100 μl of non-phenol red DMEM/10% FBS to remove extra sodium ascorbate, followed by staining with 100 μl of diluted READYPROBES® Cell Viability Imaging Kit, Blue/Green (Thermo Fisher Scientific) for 10 minutes at 37° C. to stain the nucleus of the live and dead cells. The cells were then washed 3× with non-phenol red DMEM/10% FBS. Then 100 μl of non-phenol red DMEM/10% FBS was added to each well to prepare for imaging. For the negative control, untreated cells cultured in propargyl choline and treated cells cultured without propargyl choline were included. Cells treated with BTTAA-CuSO₄ complex ([BTTAA]: [CuSO₄]=1:1, [CuSO₄]=300 μM) were also included for comparison.

For quantification of intracellular labeling of choline-containing phospholipids in live cells with LIPOFECTAMINE® RNAIMAX®, intensities were obtained from 60 random cells in three fluorescence micrographs from three biological replicates. 5 random points were acquired from each cell cytoplasm to obtain the average fluorescence of each cell. Intensity was normalized to the fluorescence intensity from the fluorescence micrographs at 30 minutes.

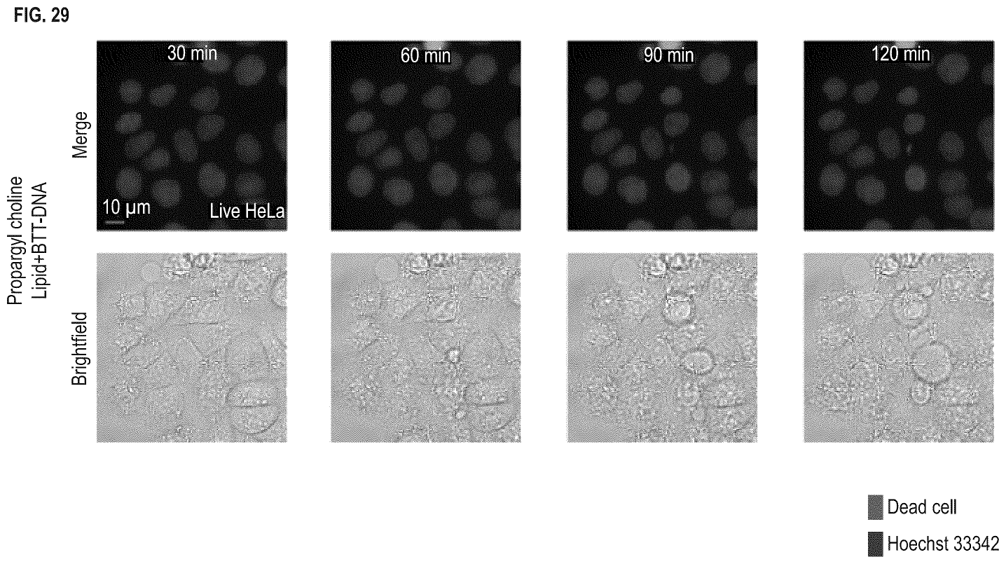

Strong fluorescence intensity was observed in the cytoplasm and membrane of cells treated with BTT-DNA (FIG. 28B), indicating that BTT-DNA ligand enables intracellular labeling of phospholipids in live cells. A timecourse labeling of phospholipids was conducted by chasing the cells for 2 hours. Some cells that were negative at 30 minutes started to show fluorescence at 1 hour and became more fluorescent at 2 hours (FIG. 28C). The fluorescence signal increased significantly over time, and the fluorescence enhancement compared to that at 30 minutes was 1.454±0.159 fold, 1.738±0.231 fold and 1.973±0.277 fold at 60 minutes, 90 minutes and 120 minutes, respectively (FIGS. 28C-28D). The cells' viability was also measured. Almost 100% of the cells were viable (FIG. 29), indicating that the BTT-DNA ligand enables the labeling and detection of intracellular phospholipids n live cells.

Additionally, to confirm the presence of free choline-containing phospholipids on the cell surface, a CuAAC reaction was conducted using Alexa Fluor 555 azide, which is a fluorescent dye with BTTAA-CuSO₄ complex for 5 minutes after intracellular phospholipids labeling. Colocalization of Alexa Fluor 555 with CalFluor 647 was observed (FIG. 28E), demonstrating the presence of the choline-containing phospholipids on the cell surface and consistent with the model in FIG. 28A. For the control sample that excludes free CuSO₄ and BTTAA, no fluorescence signal was observed (FIG. 28E), demonstrating that BTT-DNA is removed completely and that CuSO₄ and BTTAA must be added to the media for extracellular labeling. If any BTT-DNA was remaining on the surface, this was insufficient to drive the reaction on its own.

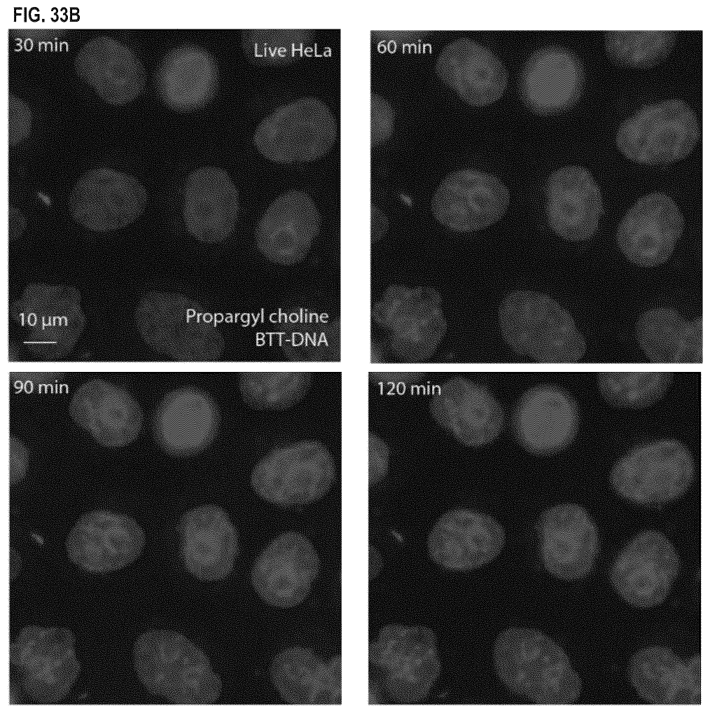

To further demonstrate that intracellular labeling of phospholipids in live cells was achieved, the cells were treated with sodium ascorbate on ice rather than 37° C. In this experiment, cells were treated with propargyl choline and the BTT-DNA and CalFluor 647 were delivered via liposome. The cells were then incubated on ice, thus making the membrane rigid and not allowing Cu(II) on the inside of the cells to convert to Cu(I), and only enabling this reduction on the cell surface (FIG. 33A). No fluorescence signal was observed after washing off the sodium ascorbate and putting the cells back at 37° C. with fresh complete medium for 2 hours (FIG. 33B). These results indicate that both the BTT-DNA ligand as well as the fluorogenic dye are intracellular after delivery and washing steps.

With the knowledge that the BTT-DNA ligand can lower the cytotoxicity caused by copper, it was then demonstrated that the BTT-DNA ligand may be used for intracellular detection of propargyl choline-labeled nascent phospholipids in live cells. Interestingly, dots near the nucleus were observed under different conditions, which might be because the fluorogenic dye aggregates after they enter the cells. The dynamic of intracellular choline-containing phospholipids was studied via CuAAC assisted by the BTT-DNA ligand for 2 hours. Surprisingly, the cells were observed to form blebs over time for cells treated with BTT-DNA, while the cells treated with copper and BTTAA did not change the morphology over time, which might be due to the CuAAC reaction assisted by our ligand. The live-cell labeling and detection enables future applications such as tracking the dynamics of other major biomolecules like global cellular transcription over time. Overall, the newly developed CuAAC accelerating ligand enables sensitive detection of biomolecules in fixed and live cells and holds great promise for further application of CuAAC for intracellular, live-cell detection.

### EQUIVALENTS

The teachings of all patents, published applications, and references cited herein are incorporated by reference in their entirety. The teachings of all patents, published applications, and references cited herein are considered to be readily accessible to and understood by a person of skill in the art at the time of application.

While example embodiments have been particularly shown and described, it will be understood by those skilled in the art that various changes in form and details may be made therein without departing from the scope of the embodiments encompassed by the appended claims.

### REFERENCES

(1) Kolb, H. C.; Finn, M. G.; Sharpless, K. B. Click Chemistry: Diverse Chemical Function from a Few Good Reactions. Angew. Chem. Int. Ed Engl. 2001, 40 (11), 2004-2021.
(2) Laughlin, S. T.; Bertozzi, C. R. Imaging the Glycome. Proc. Natl. Acad. Sci. U.S.A. 2009,106 (1), 12-17.
(3) Dieterich, D. C.; Lee, J. J.; Link, A. J.; Graumann, J.; Tirrell, D. A.; Schuman, E. M. Labeling, Detection and Identification of Newly Synthesized Proteomes with Bioorthogonal Non-Canonical Amino-Acid Tagging. Nat. Protoc. 2007, 2 (3), 532-540.
(4) Salic, A.; Mitchison, T. J. A Chemical Method for Fast and Sensitive Detection of DNA Synthesis in Vivo. Proc. Natl. Acad. Sci. U.S.A2008,105 (7), 2415-2420.
(5) Hang, H. C.; Yu, C.; Kato, D. L.; Bertozzi, C. R. A Metabolic Labeling Approach toward Proteomic Analysis of Mucin-Type O-Linked Glycosylation. Proc. Natl. Acad. Sci. U.S.A. 2003,100 (25), 14846-14851.
(6) Rostovtsev, V. V.; Green, L. G.; Fokin, V. V.; Sharpless, K. B. A Stepwise Huisgen Cycloaddition Process: Copper (I)-Catalyzed Regioselective “ligation” of Azides and Terminal Alkynes. Angew. Chem. Int. Ed. 2002, 41 (14), 2596-2599.
(7) Tornøe, C. W.; Christensen, C.; Meldal, M. Peptidotriazoles on Solid phase: [1, 2, 3]-Triazoles by Regiospecific Copper (I)-Catalyzed 1, 3-Dipolar Cycloadditions of Terminal Alkynes to Azides. J. Org. Chem. 2002, 67 (9), 3057-3064.
(8) Soriano Del Amo, D.; Wang, W.; Jiang, H.; Besanceney, C.; Yan, A. C.; Levy, M.; Liu, Y.; Marlow, F. L.; Wu, P. Biocompatible copper (I) Catalysts for in Vivo Imaging of Glycans. J. Am. Chem. Soc. 2010,132 (47), 16893-16899.
(9) Besanceney-Webler, C.; Jiang, H.; Zheng, T.; Feng, L.; Soriano del Amo, D.; Wang, W.; Klivansky, L. M.; Marlow, F. L.; Liu, Y.; Wu, P. Increasing the Efficacy of Bioorthogonal Click Reactions for Bioconjugation: A Comparative Study. Angew. Chem. Int. Ed Engl. 2011, 50 (35), 8051-8056.
(10) Hein, J. E.; Fokin, V. V. Copper-Catalyzed Azide-Alkyne Cycloaddition (CuAAC) and beyond: New Reactivity of copper (I) Acetylides. Chem. Soc. Rev. 2010, 39 (4), 1302-1315.
(11) Greenberg, M. M. Reactivity of Nucleic Acid Radicals. Adv. Phys. Org. Chem. 2016, 50, 119-202.
(12) Kennedy, D. C.; Mckay, C. S.; Legault, M. C. B.; Danielson, D. C.; Blake, J. A.; Pegoraro, A. F.; Stolow, A.; Mester, Z.; Pezacki, J. P. Cellular Consequences of Copper Complexes Used to Catalyze Bioorthogonal Click Reactions. J. Am. Chem. Soc. 2011,133 (44), 17993-18001.
(13) Uttamapinant, C.; Tangpeerachaikul, A.; Grecian, S.; Clarke, S.; Singh, U.; Slade, P.; Gee, K. R.; Ting, A. Y. Fast, Cell-Compatible Click Chemistry with Copper-Chelating Azides for Biomolecular Labeling. Angew. Chem. Int. Ed Engl. 2012, 51 (24), 5852-5856.
(14) Bevilacqua, V.; King, M.; Chaumontet, M.; Nothisen, M.; Gabillet, S.; Buisson, D.; Puente, C.; Wagner, A.; Taran, F. Copper-Chelating Azides for Efficient Click Conjugation Reactions in Complex Media. Angew. Chem. Weinheim Bergstr. Ger. 2014,126 (23), 5982-5986.
(15) Li, S.; Wang, L.; Yu, F.; Zhu, Z.; Shobaki, D.; Chen, H.; Wang, M.; Wang, J.; Qin, G.; Erasquin, U. J.; Ren, L.; Wang, Y.; Cai, C. Copper-Catalyzed Click Reaction On/in Live Cells. Chem. Sci. 2017, 8 (3), 2107-2114.
(16) Li, C.; Key, J. A.; Jia, F.; Dandapat, A.; Hur, S.; Cairo, C. W. Practical Labeling Methodology for Choline-Derived Lipids and Applications in Live Cell Fluorescence Imaging. Photochem. Photobiol. 2014, 90 (3), 686-695.
(17) Ngo, J. T.; Schuman, E. M.; Tirrell, D. A. Mutant Methionyl-tRNA Synthetase from Bacteria Enables Site-Selective N-Terminal Labeling of Proteins Expressed in Mammalian Cells. Proc. Natl. Acad. Sci. U.S.A2013, 110 (13), 4992-4997.
(18) Jao, C. Y.; Salic, A. Exploring RNA Transcription and Turnover in Vivo by Using Click Chemistry. Proc. Natl. Acad. Sci. U.S.A2008,105 (41), 15779-15784.
(19) Agard, N. J.; Prescher, J. A.; Bertozzi, C. R. A Strain-Promoted [3+2] Azide-Alkyne Cycloaddition for Covalent Modification of Biomolecules in Living Systems. J. Am. Chem. Soc. 2004,126 (46), 15046-15047.
(20) Freidel, C.; Kaloyanova, S.; Peneva, K. Chemical Tags for Site-Specific Fluorescent Labeling of Biomolecules. Amino Acids 2016, 48 (6), 1357-1372.)
(21) Beatty, K. E.; Szychowski, J.; Fisk, J. D.; Tirrell, D. A. A BODIPY-Cyclooctyne for Protein Imaging in Live Cells. Chembiochem 2011, 12 (14), 2137-2139.
(22) Plass, T.; Milles, S.; Koehler, C.; Schultz, C.; Lemke, E. A. Genetically Encoded Copper-Free Click Chemistry. Angew. Chem. Int. Ed Engl. 2011, 50 (17), 3878-3881.
(23) Braun, A. C.; Gutmann, M.; Luhmann, T.; Meinel, L. Bioorthogonal Strategies for Site-Directed Decoration of Biomaterials with Therapeutic Proteins. J. Control. Release 2018, 273, 68-85.
(24) Shieh, P.; Dien, V. T.; Beahm, B. J.; Castellano, J. M.; Wyss-Coray, T.; Bertozzi, C. R. CalFluors: A Universal Motif for Fluorogenic Azide Probes across the Visible Spectrum. J. Am. Chem. Soc. 2015,137 (22), 7145-7151.
(25) Jiang, H.; Zheng, T.; Lopez-Aguilar, A.; Feng, L.; Kopp, F.; Marlow, F. L.; Wu, P. Monitoring Dynamic Glycosylation in Vivo Using Supersensitive Click Chemistry. Bioconjug. Chem. 2014, 25 (4), 698-706.
(26) El-Sagheer, A. H.; Brown, T. Single Tube Gene Synthesis by Phosphoramidate Chemical Ligation. Chem. Commun. 2017, 53 (77), 10700-10702.
(27) Kumar, R.; El-Sagheer, A.; Tumpane, J.; Lincoln, P.; Wilhelmsson, L. M.; Brown, T. Template-Directed Oligonucleotide Strand Ligation, Covalent Intramolecular DNA Circularization and Catenation Using Click Chemistry. J. Am. Chem. Soc. 2007,129 (21), 6859-6864.
(28) Rouhanifard, S. H.; Mellis, I. A.; Dunagin, M.; Bayatpour, S.; Jiang, C. L.; Dardani, I.; Symmons, O.; Emert, B.; Torre, E.; Cote, A.; Sullivan, A.; Stamatoyannopoulos, J. A.; Raj, A. ClampFISH Detects Individual Nucleic Acid Molecules Using Click Chemistry-Based Amplification. Nat. Biotechnol. 2018. https://doi.org/10.1038/nbt.4286.
(29) Antony, T.; Thomas, T.; Shirahata, A.; Thomas, T. J. Selectivity of Polyamines on the Stability of RNA-DNA Hybrids Containing Phosphodiester and Phosphorothioate Oligodeoxyribonucleotides. Biochemistry 1999, 38 (33), 10775-10784.
(30) Braunlin, W. H.; Strick, T. J.; Record, M. T., Jr. Equilibrium Dialysis Studies of Polyamine Binding to DNA. Biopolymers 1982, 21 (7), 1301-1314.
(31) Raj, A.; Peskin, C. S.; Tranchina, D.; Vargas, D. Y.; Tyagi, S. Stochastic mRNA synthesis in mammalian cells. PLOS Biol. 2006, 4 (10), 1707-1719.
(32) Stirling, D. R. et al. CellProfiler 4: improvements in speed, utility and usability. BMC Bioinformatics 22, 433 (2021).

### ADDITIONAL REFERENCES

Berge S M, Bighley L D, Monkhouse D C. Pharmaceutical salts. J Pharm Sci. 1977 January; 66 (1): 1-19.
Bilyard M K, Becker S, Balasubramanian S. Natural, modified DNA bases. Curr Opin Chem Biol. 2020 August; 57:1-7. doi: 10.1016/j.cbpa.2020.01.014.
Bohrmann L, Burghardt T, Haynes C, Saatchi K, Hafeli U O. Aptamers used for molecular imaging and theranostics-recent developments. Theranostics. 2022 May 13; 12 (9): 4010-4050. doi: 10.7150/thno.72949.
Duffy, K., Arangundy-Franklin, S. & Holliger, P. Modified nucleic acids: replication, evolution, and next-generation therapeutics. BMC Biol 18, 112 (2020). https://doi.org/10.1186/s12915-020-00803-6
Jao C Y, Roth M, Welti R, Salic A. Metabolic labeling and direct imaging of choline phospholipids in vivo. Proc Natl Acad Sci USA. 2009 Sep. 8; 106 (36): 15332-7. doi: 10.1073/pnas.0907864106
Li, C., Guo, C., Fitzpatrick, V. et al. Design of biodegradable, implantable devices towards clinical translation. Nat Rev Mater 5, 61-81 (2020). https://doi.org/10.1038/s41578-019-0150-z
Peng Wu, David Soriano Del Amo, Wei Wang, Florence L. Marlow (2012). WO2012021390A1.
Strober W. Trypan Blue Exclusion Test of Cell Viability. Curr Protoc Immunol. 2015 Nov. 2; 111: A3.B.1-A3.B.3. doi: 10.1002/0471142735.ima03bs111.
Li, S. et al. Extent of the Oxidative Side Reactions to Peptides and Proteins During the CuAAC Reaction. Bioconjug. Chem. 27, 2315-2322 (2016).
Zhu, J. et al. Boosting Endogenous Copper (I) for Biologically Safe and Efficient Bioorthogonal Catalysis via Self-Adaptive Metal-Organic Frameworks. J. Am. Chem. Soc. 145, 1955-1963 (2023).
Mckay, C. S. & Finn, M. G. Click chemistry in complex mixtures: bioorthogonal bioconjugation. Chem. Biol. 21, 1075-1101 (2014).

Hong, V., Presolski, S. I., Ma, C. & Finn, M. G. Analysis and optimization of copper-catalyzed Azide-Alkyne cycloaddition for bioconjugation. Angew. Chem. Int. Ed Engl. 48, 9879-9883 (2009).
Beliu, G. et al. Bioorthogonal labeling with tetrazine-dyes for super-resolution microscopy. Commun Biol 2, 261 (2019).
Svatunek, D., Wilkovitsch, M., Hartmann, L., Houk, K. N. & Mikula, H. Uncovering the Key Role of Distortion in Bioorthogonal Tetrazine Tools That Defy the Reactivity/Stability Trade-Off. J. Am. Chem. Soc. 144, 8171-8177 (2022).
Liu, J., Xu, Y., Stoleru, D. & Salic, A. Imaging protein synthesis in cells and tissues ith an alkyne analog of puromycin. Proc. Natl. Acad. Sci. U.S.A 109, 413-418 (2012).

What is claimed is:

1. A system for performing Cu(I)-catalyzed azide-alkyne cycloaddition (CuAAC) inside or on a surface of a cell, comprising:
a) a CuAAC-accelerating ligand comprising a polynucleotide and one or more copper chelators; and
b) a delivery reagent.

2. The system of claim 1, wherein the cell is a live cell.

3. The system of claim 1, wherein the delivery reagent comprises a transfection reagent, a reversible pore-forming toxin, or both.

4. The system of claim 3, wherein the transfection reagent comprises a lipofection reagent.

5. The system of claim 3, wherein the reversible pore-forming toxin comprises Streptolysin-O (SLO).

6. The system of claim 1, wherein the polynucleotide of the ligand is hybridized to an oligonucleotide.

7. The system of claim 6, wherein the oligonucleotide is complementary to at least a portion of the polynucleotide of the ligand.

8. The system of claim 1, further comprising a target comprising an alkyne-containing compound.

9. The system of claim 8, wherein the alkyne-containing compound is selected from:
a) an alkyne-derivatized thymidine analog;
b) an alkyne-derivatized uridine analog;
c) an alkyne-derivatized methionine analog;
d) an alkyne-derivatized puromycin analog;
e) an alkyne-derivatized monosaccharide;
f) an alkyne-derivatized choline;
g) a 5′ alkyne DNA probe;
h) a library of alkyne-containing compounds; or
any combination of the foregoing.

10. The system of claim 9, wherein:
a) the alkyne-derivatized thymidine analog comprises 5-ethynyl-2′-deoxyuridine (EdU);
b) the alkyne-derivatized uridine analog comprises 5-ethynyl uridine (EU);
c) the alkyne-derivatized methionine analog comprises L-homopropargyl (L-HPG);
d) the alkyne-derivatized puromycin analog comprises O-propargylpuromycin (OPP);
e) the alkyne-derivatized monosaccharide comprises N-(4-pentynoyl) mannosamine (Ac₄MaNAl);
f) the alkyne-derivatized choline comprises propargyl choline; or
g) any combination of the foregoing.

11. The system of claim 1, further comprising a payload comprising an azide.

12. The system of claim 11, wherein the payload comprises a detectable molecule.

13. The system of claim 12, wherein the payload comprises a fluorogenic azide.

14. The system of claim 1, further comprising sodium ascorbate.

15. The system of claim 1, further comprising a nucleic acid splint.

16. The system of claim 1, wherein the polynucleotide of the ligand:
a) comprises a single-stranded DNA, a single-stranded RNA, a single-stranded XNA, a single-stranded modified polynucleotide, an aptamer, or a combination thereof;
b) is complementary to one or more other polynucleotides selected from: a template, a target, a payload, and a combination thereof;
c) binds a polypeptide;
d) is conjugated to at least one of: a nanoparticle, a small molecule, a liposome, an antibody or antigen-binding fragment thereof, a detectable molecule, and a combination thereof; or
e) any combination of the foregoing.

17. The system of claim 1, wherein the one or more copper chelators of the ligand comprise 2-(4-((Bis((1-(tert-butyl)-1H-1,2,3-triazol-4-yl)methyl)amino)methyl)-1H-1,2,3-triazol-1-yl) (BTT).

18. The system of claim 1, wherein the ligand comprises BTT-DNA or BTT_{(1,2)}-DNA.

19. A method of performing a Cu(I)-catalyzed azide-alkyne cycloaddition (CuAAC) inside or on a surface of a cell, comprising contacting the cell with the system of claim 1.

20. The method of claim 19, wherein the cell is contacted with, in order:
a) the delivery reagent, wherein the delivery reagent comprises a reversible pore-forming toxin; a payload comprising an azide and a target comprising an alkyne-containing compound; the ligand; and sodium ascorbate;
b) the target comprising the alkyne-containing compound; the delivery agent, wherein the delivery agent comprises the reversible pore-forming toxin; the payload comprising the azide; the ligand; and sodium ascorbate; and
c) the ligand, wherein the polynucleotide of the ligand is hybridized to an oligonucleotide; the delivery reagent, wherein the delivery reagent comprises a transfection reagent, and wherein the transfection agent is complexed with the ligand hybridized to the oligonucleotide; the delivery agent, wherein the delivery agent comprises the reversible pore-forming toxin; the payload comprising the azide; the target comprising the alkyne-containing compound; and sodium ascorbate.


SEQUENCE LISTING (15 sequences)

SEQ ID NO: 1 - DNA, 15 bp, synthetic construct
  source 1..15: mol_type = other DNA
  modified_base 15: mod_base = OTHER; note = 3' Azide
ggtgctcttcgtcca

SEQ ID NO: 2 - DNA, 15 bp, synthetic construct
  source 1..15: mol_type = other DNA
  modified_base 15: mod_base = OTHER; note = 3' Azide
gcagagacactattg

SEQ ID NO: 3 - DNA, 15 bp, synthetic construct
  source 1..15: mol_type = other DNA
  modified_base 1: mod_base = OTHER; note = 5' Hexynyl
caaacacaactcctg

SEQ ID NO: 4 - DNA, 20 bp, synthetic construct
  source 1..20: mol_type = other DNA
  modified_base 1: mod_base = OTHER; note = 5' Hexynyl
caaacacaactcctggtcga

SEQ ID NO: 5 - DNA, 20 bp, synthetic construct
  source 1..20: mol_type = other DNA
  modified_base 1: mod_base = OTHER; note = 5' Hexynyl
aaacacaactcctggtcgag

SEQ ID NO: 6 - DNA, 20 bp, synthetic construct
  source 1..20: mol_type = other DNA
  modified_base 1: mod_base = OTHER; note = 5' Hexynyl
aacacaactcctggtcgagg

SEQ ID NO: 7 - DNA, 21 bp, synthetic construct
  source 1..21: mol_type = other DNA
  modified_base 1: mod_base = OTHER; note = 5' Hexynyl
tcaaacacaactcctggtcga

SEQ ID NO: 8 - DNA, 22 bp, synthetic construct
  source 1..22: mol_type = other DNA
  modified_base 1: mod_base = OTHER; note = 5' Hexynyl
ttcaaacacaactcctggtcga

SEQ ID NO: 9 - DNA, 23 bp, synthetic construct
  source 1..23: mol_type = other DNA
  modified_base 1: mod_base = OTHER; note = 5' Hexynyl
tttcaaacacaactcctggtcga

SEQ ID NO: 10 - DNA, 24 bp, synthetic construct
  source 1..24: mol_type = other DNA
  modified_base 1: mod_base = OTHER; note = 5' Hexynyl
ttttcaaacacaactcctggtcga

SEQ ID NO: 11 - DNA, 40 bp, synthetic construct
  source 1..40: mol_type = other DNA
cgacctcgaccaggagttgtgtttgtggacgaagagcacc

SEQ ID NO: 12 - DNA, 83 bp, synthetic construct
  source 1..83: mol_type = other DNA
cgacctcgaccaggagttgtgtttgtggacgaagagcaccaaacgacctcgaccaggagttgtgtttgtggacgaagagcacc

SEQ ID NO: 13 - DNA, 126 bp, synthetic construct
  source 1..126: mol_type = other DNA
cgacctcgaccaggagttgtgtttgtggacgaagagcaccaaacgacctcgaccaggagttgtgtttgtggacgaagagcaccaaacgacctcgaccaggagttgtgtttgtggacgaagagcacc

SEQ ID NO: 14 - RNA, 40 nt, synthetic construct
  source 1..40: mol_type = other RNA
cgacctcgaccaggagttgtgtttgtggacgaagagcacc

SEQ ID NO: 15 - DNA, 15 bp, synthetic construct
  source 1..15: mol_type = other DNA
tggacgaagagcacc